SEQUENCE LISTING (365 sequences)

SEQ ID NO: 1 - DNA, 360 bp, Homo sapiens
  source 1..360: mol_type = other DNA
gaggtgcagctggtgcagtctggagcagaggtgaaaaagcccggggagtctctgaagatctcctgtaagggttctggatacagctttaccagttactggatcgtctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacaagatataactggaactacggggtttgactactggggccagggaaccctggtcaccgtctcctca

SEQ ID NO: 2 - protein, 120 aa, Homo sapiens
EVQLVQSGAEVKKPGESLKISCKGSGYSFTSYWIVWVRQMPGKGLEWMGIIYPGDSDTRYSPSFQGQVTISADKSISTAYLQWSSLKASDTAMYYCARQDITGTTGFDYWGQGTLVTVSS

SEQ ID NO: 3 - DNA, 24 bp, Homo sapiens
  source 1..24: mol_type = other DNA
ggatacagctttaccagttactgg

SEQ ID NO: 4 - protein, 8 aa, Homo sapiens
GYSFTSYW

SEQ ID NO: 5 - DNA, 24 bp, Homo sapiens
  source 1..24: mol_type = other DNA
atctatcctggtgactctgatacc

SEQ ID NO: 6 - protein, 8 aa, Homo sapiens
IYPGDSDT

SEQ ID NO: 7 - DNA, 39 bp, Homo sapiens
  source 1..39: mol_type = other DNA
gcgagacaagatataactggaactacggggtttgactac

SEQ ID NO: 8 - protein, 13 aa, Homo sapiens
ARQDITGTTGFDY

SEQ ID NO: 9 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattaggaactatttgaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttatttctgtcaacagagttacagtaccccattcactttcggccctgggaccaaagtggatatcaaacga

SEQ ID NO: 10 - protein, 108 aa, Homo sapiens
DIQMTQSPSSLSASVGDRVTITCRASQSIRNYLNWYQQKPGKAPKLLIYAASSLQSGVPSRFSGSGSGTDFTLTISSLQPEDFATYFCQQSYSTPFTFGPGTKVDIKR

SEQ ID NO: 11 - DNA, 18 bp, Homo sapiens
  source 1..18: mol_type = other DNA
cagagcattaggaactat

SEQ ID NO: 12 - protein, 6 aa, Homo sapiens
QSIRNY

SEQ ID NO: 13-14 - skipped (2 entries)

SEQ ID NO: 15 - DNA, 27 bp, Homo sapiens
  source 1..27: mol_type = other DNA
caacagagttacagtaccccattcact

SEQ ID NO: 16 - protein, 9 aa, Homo sapiens
QQSYSTPFT

SEQ ID NO: 17 - DNA, 369 bp, Homo sapiens
  source 1..369: mol_type = other DNA
caggtgcagctggtggagtctgggggagacgtggtccagcctgggaggtccctgcgactctcctgttcaggcactggattcaccttcagtagctatgccatgcactgggtccgccaggctccaggcaagggactggaatgggtggcacttatatcatatgatggaagtaataaatattatggagactccgtgaagggccgattcaccgtctccagagacaattccaagaacacgctgtctctgcaaatgaacagcctgaaaactgaggacacggcgatatattactgtgcgaaagagaggctttttggagtggtctcttattacggtatggacgtctggggccaagggaccacggtcaccgtctcctca

SEQ ID NO: 18 - protein, 123 aa, Homo sapiens
QVQLVESGGDVVQPGRSLRLSCSGTGFTFSSYAMHWVRQAPGKGLEWVALISYDGSNKYYGDSVKGRFTVSRDNSKNTLSLQMNSLKTEDTAIYYCAKERLFGVVSYYGMDVWGQGTTVTVSS

SEQ ID NO: 19 - DNA, 24 bp, Homo sapiens
  source 1..24: mol_type = other DNA
ggattcaccttcagtagctatgcc

SEQ ID NO: 20 - protein, 8 aa, Homo sapiens
GFTFSSYA

SEQ ID NO: 21 - DNA, 24 bp, Homo sapiens
  source 1..24: mol_type = other DNA
atatcatatgatggaagtaataaa

SEQ ID NO: 22 - protein, 8 aa, Homo sapiens
ISYDGSNK

SEQ ID NO: 23 - DNA, 48 bp, Homo sapiens
  source 1..48: mol_type = other DNA
gcgaaagagaggctttttggagtggtctcttattacggtatggacgtc

SEQ ID NO: 24 - protein, 16 aa, Homo sapiens
AKERLFGVVSYYGMDV

SEQ ID NO: 25 - DNA, 324 bp, Homo sapiens
  source 1..324: mol_type = other DNA
gacatccagatgacccagtctccatcctccctgtctgcatctgtaggagacagagtcaccatcacttgccaggcgagtcaggacattaatagggatctaaattggtatcagcagaaatcagggaaaggccccaaactcctgatctacgatgcatccaatttggaaacaggggtcccatcaaggttcagtggaaatagatttgggacagattttactttcaccatcagcagactgcagcctgaagatattgcaacatatttctgtcaacagtataaaaatctcccgtacacttttggccaggggaccaagctggagatcaaacga

SEQ ID NO: 26 - protein, 108 aa, Homo sapiens
DIQMTQSPSSLSASVGDRVTITCQASQDINRDLNWYQQKSGKGPKLLIYDASNLETGVPSRFSGNRFGTDFTFTISRLQPEDIATYFCQQYKNLPYTFGQGTKLEIKR

SEQ ID NO: 27 - DNA, 18 bp, Homo sapiens
  source 1..18: mol_type = other DNA
caggacattaatagggat

SEQ ID NO: 28 - protein, 6 aa, Homo sapiens
QDINRD

SEQ ID NO: 29-30 - skipped (2 entries)

SEQ ID NO: 31 - DNA, 27 bp, Homo sapiens
  source 1..27: mol_type = other DNA
caacagtataaaaatctcccgtacact

SEQ ID NO: 32 - protein, 9 aa, Homo sapiens
QQYKNLPYT

SEQ ID NO: 33 - DNA, 357 bp, Homo sapiens
  source 1..357: mol_type = other DNA
cagctgcagctgcaggagtcgggcccaggactggtgaagccttcggagaccctgtccctcacctgcactgtctctggtggctccatcagcagtagtagttactactggggctggatccgccagcccccagggaagggcctggagtggattgggattatctattatagtgggagcacctactacaacccgtccctcaagagtcgagtcaccatttccgtagacacgtccaagaaccagttctccctgaagctgagctctgtgaccgccgcagacacggctgtgtattactgtgcgagacaggacagggaggccctctttgactactggggccagggaaccctggtcaccgtctcctca

SEQ ID NO: 34 - protein, 119 aa, Homo sapiens
QLQLQESGPGLVKPSETLSLTCTVSGGSISSSSYYWGWIRQPPGKGLEWIGIIYYSGSTYYNPSLKSRVTISVDTSKNQFSLKLSSVTAADTAVYYCARQDREALFDYWGQGTLVTVSS

SEQ ID NO: 35 - DNA, 30 bp, Homo sapiens
  source 1..30: mol_type = other DNA
ggtggctccatcagcagtagtagttactac

SEQ ID NO: 36 - protein, 10 aa, Homo sapiens
GGSISSSSYY

SEQ ID NO: 37 - DNA, 21 bp, Homo sapiens
  source 1..21: mol_type = other DNA
atctattatagtgggagcacc

SEQ ID NO: 38 - protein, 7 aa, Homo sapiens
IYYSGST

SEQ ID NO: 39 - DNA, 33 bp, Homo sapiens
  source 1..33: mol_type = other DNA
gcgagacaggacagggaggccctctttgactac

SEQ ID NO: 40 - protein, 11 aa, Homo sapiens
ARQDREALFDY

SEQ ID NO: 41 - DNA, 321 bp, Homo sapiens
  source 1..321: mol_type = other DNA
gaaattgtgctgactcagtctccagactttcagtctgtgactccaaaggagaaagtcaccatcacctgccgggccagtcagcgcattggtagtagcttacactggtaccagcagaaaccagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcatcagagtagtactttacccaccttcggccaagggacacgactggagattaaacga

SEQ ID NO: 42 - protein, 107 aa, Homo sapiens
EIVLTQSPDFQSVTPKEKVTITCRASQRIGSSLHWYQQKPDQSPKLLIKYASQSFSGVPSRFSGSGSGTDFTLTINSLEAEDAATYYCHQSSTLPTFGQGTRLEIKR

SEQ ID NO: 43 - DNA, 18 bp, Homo sapiens
  source 1..18: mol_type = other DNA
cagcgcattggtagtagc

SEQ ID NO: 44 - protein, 6 aa, Homo sapiens
QRIGSS

SEQ ID NO: 45-46 - skipped (2 entries)

SEQ ID NO: 47 - DNA, 24 bp, Homo sapiens
  source 1..24: mol_type = other DNA
catcagagtagtactttacccacc

SEQ ID NO: 48 - protein, 8 aa, Homo sapiens
HQSSTLPT

SEQ ID NO: 49 - DNA, 375 bp, Homo sapiens
  source 1..375: mol_type = other DNA
gaagtgcagctggtggagtctgggggaggcctggtacagcctggcaggtccctgagactctcctgtgcagcctctggattcaggtttgacgattatgccatgcactgggtccgacaagctccagggaagggcctggaatgggtctcaggtattaattggaatagtggtggcaaaggctatgcggactctgtgcagggccgattcaccatctccagagacaacgccaagaactccctttatctgcaaatgaacagtctgagaactgaggacacggccttgtattattgtgcaaaagataggggtatagcagctcgtcttctctctcgtgatgcttttgatatgtggggccaagggacaatggtcaccgtctcttca

SEQ ID NO: 50 - protein, 125 aa, Homo sapiens
EVQLVESGGGLVQPGRSLRLSCAASGFRFDDYAMHWVRQAPGKGLEWVSGINWNSGGKGYADSVQGRFTISRDNAKNSLYLQMNSLRTEDTALYYCAKDRGIAARLLSRDAFDMWGQGTMVTVSS

SEQ ID NO: 51 - DNA, 24 bp, Homo sapiens
  source 1..24: mol_type = other DNA
ggattcaggtttgacgattatgcc

SEQ ID NO: 52 - protein, 8 aa, Homo sapiens
GFRFDDYA

SEQ ID NO: 53 - DNA, 24 bp, Homo sapiens
  source 1..24: mol_type = other DNA
attaattggaatagtggtggcaaa

SEQ ID NO: 54 - protein, 8 aa, Homo sapiens
INWNSGGK

SEQ ID NO: 55 - DNA, 54 bp, Homo sapiens
  source 1..54: mol_type = other DNA
gcaaaagataggggtatagcagctcgtcttctctctcgtgatgcttttgatatg

SEQ ID NO: 56 - protein, 18 aa, Homo sapiens
AKDRGIAARLLSRDAFDM

SEQ ID NO: 57 - DNA, 324 bp, Homo sapiens
  source 1..324: mol_type = other DNA
gacatccagttgacccagtctccatccttcctgtctgcatctgtaggagacagagtcaccatcacttgctgggccagtcaggacgttagcagttatttagcctggtatcagcaaaaaccagggaaatcccctaagctcctaatctttgctgcatccactttgcaaggtgggatcccatcaaggttcagcggcagtggatctgggacagaattcactctcacaatcagcagcctgcggcctgaagattttgcaacttattactgtcaacaccttaatacttacccgtacacttttggccaggggaccaagctggagatcaaacga

SEQ ID NO: 58 - protein, 108 aa, Homo sapiens
DIQLTQSPSFLSASVGDRVTITCWASQDVSSYLAWYQQKPGKSPKLLIFAASTLQGGIPSRFSGSGSGTEFTLTISSLRPEDFATYYCQHLNTYPYTFGQGTKLEIKR

SEQ ID NO: 59 - DNA, 18 bp, Homo sapiens
  source 1..18: mol_type = other DNA
caggacgttagcagttat

SEQ ID NO: 60 - protein, 6 aa, Homo sapiens
QDVSSY

SEQ ID NO: 61-62 - skipped (2 entries)

SEQ ID NO: 63 - DNA, 27 bp, Homo sapiens
  source 1..27: mol_type = other DNA
caacaccttaatacttacccgtacact

SEQ ID NO: 64 - protein, 9 aa, Homo sapiens
QHLNTYPYT

SEQ ID NO: 65 - DNA, 363 bp, Homo sapiens
  source 1..363: mol_type = other DNA
caggtgcagctggtggagtctgggggaggcgtggtccagtctgggaggtccctgagactctcctgtgcagcgtcttcattcaccttcagtagctatggcatgcactgggtccgccagtctccaggcaaggggctggagtgggtggcagttataagttatgatggaagtaataaatactatggagacttcgtgaggggccgattcaccatctccagagacaattccaagaacacgctgtatctgcaaatgaacagcctgagagccgaggacacggctatgtattattgtgcgagagaagttcgtcgctactattattacggtatggacgtctggggccaagggaccacggtcaccgtctcctca

SEQ ID NO: 66 - protein, 121 aa, Homo sapiens
QVQLVESGGGVVQSGRSLRLSCAASSFTFSSYGMHWVRQSPGKGLEWVAVISYDGSNKYYGDFVRGRFTISRDNSKNTLYLQMNSLRAEDTAMYYCAREVRRYYYYGMDVWGQGTTVTVSS

SEQ ID NO: 67 - DNA, 24 bp, Homo sapiens
  source 1..24: mol_type = other DNA
tcattcaccttcagtagctatggc

SEQ ID NO: 68 - protein, 8 aa, Homo sapiens
SFTFSSYG

SEQ ID NO: 69 - DNA, 24 bp, Homo sapiens
  source 1..24: mol_type = other DNA
ataagttatgatggaagtaataaa

SEQ ID NO: 70 - protein, 8 aa, Homo sapiens
ISYDGSNK

SEQ ID NO: 71 - DNA, 42 bp, Homo sapiens
  source 1..42: mol_type = other DNA
gcgagagaagttcgtcgctactattattacggtatggacgtc

SEQ ID NO: 72 - protein, 14 aa, Homo sapiens
AREVRRYYYYGMDV

SEQ ID NO: 73 - DNA, 324 bp, Homo sapiens
  source 1..324: mol_type = other DNA
gacatccagatgacccagtctccatcctccctgtctgcatctgtaggagacagagtcaccatcacttgccaggcgagtcaggacattagtaattatttaaattggtatctgcagaaaccagggaaagcccctaagctcctgatctccgatgcatccaatttggaaacaggggtcccatcaaggttcagtggaagtggatctgggacagattttactttcaccatcagcagcctgcagcctgaagatattgcaacatattactgtcaacagtataataatctcccgctcactttcggcggagggaccaaggtggagatcaaacga

SEQ ID NO: 74 - protein, 108 aa, Homo sapiens
DIQMTQSPSSLSASVGDRVTITCQASQDISNYLNWYLQKPGKAPKLLISDASNLETGVPSRFSGSGSGTDFTFTISSLQPEDIATYYCQQYNNLPLTFGGGTKVEIKR

SEQ ID NO: 75 - DNA, 18 bp, Homo sapiens
  source 1..18: mol_type = other DNA
caggacattagtaattat

SEQ ID NO: 76 - protein, 6 aa, Homo sapiens
QDISNY

SEQ ID NO: 77-78 - skipped (2 entries)

SEQ ID NO: 79 - DNA, 27 bp, Homo sapiens
  source 1..27: mol_type = other DNA
caacagtataataatctcccgctcact

SEQ ID NO: 80 - protein, 9 aa, Homo sapiens
QQYNNLPLT

SEQ ID NO: 81 - DNA, 372 bp, Homo sapiens
  source 1..372: mol_type = other DNA
gaagtgcagctggtggagtctgggggaggcttggtacagcctggcaggtccctgagactctcctgtgcagtctctggattcacctttgatgattatgccatgcactgggtccggcaagctccagggaagggcctggagtgggtctcaggtattagttggaatagtggtgacatagactatgcggactctgtgaagggccgattcaccatttccagagacaacgccaagaactccctgtatctgcaaatgaacagtctgagagctgaggacacggccttgtattactgtgcaaaagataccctctcagggactggaactacgtggtactattttgactactggggccagggaaccctggtcaccgtctcctca

SEQ ID NO: 82 - protein, 124 aa, Homo sapiens
EVQLVESGGGLVQPGRSLRLSCAVSGFTFDDYAMHWVRQAPGKGLEWVSGISWNSGDIDYADSVKGRFTISRDNAKNSLYLQMNSLRAEDTALYYCAKDTLSGTGTTWYYFDYWGQGTLVTVSS

SEQ ID NO: 83 - DNA, 24 bp, Homo sapiens
  source 1..24: mol_type = other DNA
ggattcacctttgatgattatgcc

SEQ ID NO: 84 - protein, 8 aa, Homo sapiens
GFTFDDYA

SEQ ID NO: 85 - DNA, 24 bp, Homo sapiens
  source 1..24: mol_type = other DNA
attagttggaatagtggtgacata

SEQ ID NO: 86 - protein, 8 aa, Homo sapiens
ISWNSGDI

SEQ ID NO: 87 - DNA, 51 bp, Homo sapiens
  source 1..51: mol_type = other DNA
gcaaaagataccctctcagggactggaactacgtggtactattttgactac

SEQ ID NO: 88 - protein, 17 aa, Homo sapiens
AKDTLSGTGTTWYYFDY

SEQ ID NO: 89 - DNA, 324 bp, Homo sapiens
  source 1..324: mol_type = other DNA
gacatccagttgacccagtctccatccttcctgtctgcatctgtaggagacagagtcaccatcacttgctgggccagtcagggtattagcagttatttaatctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacaggtgaatagttaccctctcactttcggcggagggaccaaggtggagatcaaacga

SEQ ID NO: 90 - protein, 108 aa, Homo sapiens
DIQLTQSPSFLSASVGDRVTITCWASQGISSYLIWYQQKPGKAPKLLIYAASTLQSGVPSRFSGSGSGTEFTLTISSLQPEDFATYYCQQVNSYPLTFGGGTKVEIKR

SEQ ID NO: 91 - DNA, 18 bp, Homo sapiens
  source 1..18: mol_type = other DNA
cagggtattagcagttat

SEQ ID NO: 92 - protein, 6 aa, Homo sapiens
QGISSY

SEQ ID NO: 93-94 - skipped (2 entries)

SEQ ID NO: 95 - DNA, 27 bp, Homo sapiens
  source 1..27: mol_type = other DNA
caacaggtgaatagttaccctctcact

SEQ ID NO: 96 - protein, 9 aa, Homo sapiens
QQVNSYPLT

SEQ ID NO: 97 - DNA, 369 bp, Homo sapiens
  source 1..369: mol_type = other DNA
caggtgcagctggtggagtctgggggaggcgtggtccagcctgggaggtccctgagactctcctgtgcagcctctggattcaccttcagtagttatggcatgcactgggtccgccaggctccaggcaagggactggagtggatggcagttatatcatatgatggaagtaataaatattatgcagactccgtgaagggccgattcaccatctccagagacaattccaagaacacgctgtttctgcaaatgaacagcctgagacctgaagacacggctgtatattactgtgcgcaagatggcagctcggcgatttactatttctacggtatggacgtctggggccaagggaccacggtcaccgtctcctca

SEQ ID NO: 98 - protein, 123 aa, Homo sapiens
QVQLVESGGGVVQPGRSLRLSCAASGFTFSSYGMHWVRQAPGKGLEWMAVISYDGSNKYYADSVKGRFTISRDNSKNTLFLQMNSLRPEDTAVYYCAQDGSSAIYYFYGMDVWGQGTTVTVSS

SEQ ID NO: 99 - DNA, 24 bp, Homo sapiens
  source 1..24: mol_type = other DNA
ggattcaccttcagtagttatggc

SEQ ID NO: 100 - protein, 8 aa, Homo sapiens
GFTFSSYG

SEQ ID NO: 101 - DNA, 24 bp, Homo sapiens
  source 1..24: mol_type = other DNA
atatcatatgatggaagtaataaa

SEQ ID NO: 102 - protein, 8 aa, Homo sapiens
ISYDGSNK

SEQ ID NO: 103 - DNA, 48 bp, Homo sapiens
  source 1..48: mol_type = other DNA
gcgcaagatggcagctcggcgatttactatttctacggtatggacgtc

SEQ ID NO: 104 - protein, 16 aa, Homo sapiens
AQDGSSAIYYFYGMDV

SEQ ID NO: 105 - DNA, 324 bp, Homo sapiens
  source 1..324: mol_type = other DNA
gacatccagatgacccagtctccatcctcactgtctgcatctataggagacagagtcaccatcacttgtcgggcgagtcaggacatcaacaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccttcagtataatagttaccatcccacttttggccaggggaccaagctggagatcaaacga

SEQ ID NO: 106 - protein, 108 aa, Homo sapiens
DIQMTQSPSSLSASIGDRVTITCRASQDINNYLAWFQQKPGKAPKSLIYAASSLQSGVPSKFSGSGSGTDFTLTISSLQPEDFATYYCLQYNSYHPTFGQGTKLEIKR

SEQ ID NO: 107 - DNA, 18 bp, Homo sapiens
  source 1..18: mol_type = other DNA
caggacatcaacaattat

SEQ ID NO: 108 - protein, 6 aa, Homo sapiens
QDINNY

SEQ ID NO: 109-110 - skipped (2 entries)

SEQ ID NO: 111 - DNA, 27 bp, Homo sapiens
  source 1..27: mol_type = other DNA
cttcagtataatagttaccatcccact

SEQ ID NO: 112 - protein, 9 aa, Homo sapiens
LQYNSYHPT

SEQ ID NO: 113 - DNA, 363 bp, Homo sapiens
  source 1..363: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagctatatggtatgatggaagtaataaatattatgcagactccgtgaagggccgattcaccatctccagagacaattccaagaacacgctgtatctgcaaatgaacagcctgagagccgaggacacggctgtgtatttctgtgcgagaggggaacattactatggttcggggccgttcgacccctggggccagggaaccctggtcaccgtctcctca

SEQ ID NO: 114 - protein, 121 aa, Homo sapiens
QVQLVESGGGVVQPGRSLRLSCAASGFTFSSYGMHWVRQAPGKGLEWVAAIWYDGSNKYYADSVKGRFTISRDNSKNTLYLQMNSLRAEDTAVYFCARGEHYYGSGPFDPWGQGTLVTVSS

SEQ ID NO: 115 - DNA, 24 bp, Homo sapiens
  source 1..24: mol_type = other DNA
ggattcaccttcagtagctatggc

SEQ ID NO: 116 - protein, 8 aa, Homo sapiens
GFTFSSYG

SEQ ID NO: 117 - DNA, 24 bp, Homo sapiens
  source 1..24: mol_type = other DNA
atatggtatgatggaagtaataaa

SEQ ID NO: 118 - protein, 8 aa, Homo sapiens
IWYDGSNK

SEQ ID NO: 119 - DNA, 42 bp, Homo sapiens
  source 1..42: mol_type = other DNA
gcgagaggggaacattactatggttcggggccgttcgacccc

SEQ ID NO: 120 - protein, 14 aa, Homo sapiens
ARGEHYYGSGPFDP

SEQ ID NO: 121 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattagcaactatttaaattggtatcagcagaaaccagggaaagcccctaagctcctgatctttgctgcatccagtttacaaagtggggtcccatcaaggttcagtggcagtggatctgggacagatttcactctcaccatcagcagtctgcaacctgaagattttgcaacttactactgtcaacagagttacagttccccgctcactttcggcggagggaccaaggtggagatcaaacga

SEQ ID NO: 122 - protein, 108 aa, Homo sapiens
DIQMTQSPSSLSASVGDRVTITCRASQSISNYLNWYQQKPGKAPKLLIFAASSLQSGVPSRFSGSGSGTDFTLTISSLQPEDFATYYCQQSYSSPLTFGGGTKVEIKR

SEQ ID NO: 123 - DNA, 18 bp, Homo sapiens
  source 1..18: mol_type = other DNA
cagagcattagcaactat

SEQ ID NO: 124 - protein, 6 aa, Homo sapiens
QSISNY

SEQ ID NO: 125-126 - skipped (2 entries)

SEQ ID NO: 127 - DNA, 27 bp, Homo sapiens
  source 1..27: mol_type = other DNA
caacagagttacagttccccgctcact

SEQ ID NO: 128 - protein, 9 aa, Homo sapiens
QQSYSSPLT

SEQ ID NO: 129 - DNA, 360 bp, Homo sapiens
  source 1..360: mol_type = other DNA
caggtgcagctgcaggagtcggggccaggactggtgaagccttcacagaccctgtccctcacctgcactgtctcaggtggctccatcagcagttttggttactactggagctggatccgccagcacccagggaagggcctggagtggattgggtacatctattacagtgggagcatcgactacaacccgtccctcaagagtcgaattaccatatcagtcgacacgtctaagaaccagttctccctgaagctgagctctgtgactgccgcggacacggccgtgtattactgtgcgagagaaagggactacggtgactactttgactactggggccagggaaccctggtcaccgtctcctca

SEQ ID NO: 130 - protein, 120 aa, Homo sapiens
QVQLQESGPGLVKPSQTLSLTCTVSGGSISSFGYYWSWIRQHPGKGLEWIGYIYYSGSIDYNPSLKSRITISVDTSKNQFSLKLSSVTAADTAVYYCARERDYGDYFDYWGQGTLVTVSS

SEQ ID NO: 131 - DNA, 30 bp, Homo sapiens
  source 1..30: mol_type = other DNA
ggtggctccatcagcagttttggttactac

SEQ ID NO: 132 - protein, 10 aa, Homo sapiens
GGSISSFGYY

SEQ ID NO: 133 - DNA, 21 bp, Homo sapiens
  source 1..21: mol_type = other DNA
atctattacagtgggagcatc

SEQ ID NO: 134 - protein, 7 aa, Homo sapiens
IYYSGSI

SEQ ID NO: 135 - DNA, 36 bp, Homo sapiens
  source 1..36: mol_type = other DNA
gcgagagaaagggactacggtgactactttgactac

SEQ ID NO: 136 - protein, 12 aa, Homo sapiens
ARERDYGDYFDY

SEQ ID NO: 137 - DNA, 324 bp, Homo sapiens
  source 1..324: mol_type = other DNA
gaaatagtgatgacgcagtctccagccaccctgtctgtctctccaggggaaagagccaccctctcctgcagggccagtcagagtgttagcagcaacttagcctggtaccagcagaaacctggccaggctcccaggctcctcatctatggtgcatccaccagggccactggtgtcccagccaggttcagtggcagtgggtctgggacagagttcactctcaccatcagcagtttgcagtctgaggattttgcagtttattcctgtcagcagtataataactggcctctcactttcggcggagggaccaaggtggagatcaaacga

SEQ ID NO: 138 - protein, 108 aa, Homo sapiens
EIVMTQSPATLSVSPGERATLSCRASQSVSSNLAWYQQKPGQAPRLLIYGASTRATGVPARFSGSGSGTEFTLTISSLQSEDFAVYSCQQYNNWPLTFGGGTKVEIKR

SEQ ID NO: 139 - DNA, 18 bp, Homo sapiens
  source 1..18: mol_type = other DNA
cagagtgttagcagcaac

SEQ ID NO: 140 - protein, 6 aa, Homo sapiens
QSVSSN

SEQ ID NO: 141-142 - skipped (2 entries)

SEQ ID NO: 143 - DNA, 27 bp, Homo sapiens
  source 1..27: mol_type = other DNA
cagcagtataataactggcctctcact

SEQ ID NO: 144 - protein, 9 aa, Homo sapiens
QQYNNWPLT

SEQ ID NO: 145 - DNA, 372 bp, Homo sapiens
  source 1..372: mol_type = other DNA
caggtgcagctggtggagtctgggggaggcgtggtccagcctgggaggtccctgagactctcctgtgcagcgtctggattcaccttcagtagctatggcatacactgggtccgccaggctccaggcaaggggctggagtgggtggcacttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatcaggattactatggttcggggagttcctacggtatggacgtctggggccaagggaccacggtcaccgtctcctca

SEQ ID NO: 146 - protein, 124 aa, Homo sapiens
QVQLVESGGGVVQPGRSLRLSCAASGFTFSSYGIHWVRQAPGKGLEWVALIWYDGSNKYYADSVKGRFTISRDNSKNTLYLQMNSLRAEDTAVYYCARDQDYYGSGSSYGMDVWGQGTTVTVSS

SEQ ID NO: 147 - DNA, 24 bp, Homo sapiens
  source 1..24: mol_type = other DNA
ggattcaccttcagtagctatggc

SEQ ID NO: 148 - protein, 8 aa, Homo sapiens
GFTFSSYG

SEQ ID NO: 149 - DNA, 24 bp, Homo sapiens
  source 1..24: mol_type = other DNA
atatggtatgatggaagtaataaa

SEQ ID NO: 150 - protein, 8 aa, Homo sapiens
IWYDGSNK

SEQ ID NO: 151 - DNA, 51 bp, Homo sapiens
  source 1..51: mol_type = other DNA
gcgagagatcaggattactatggttcggggagttcctacggtatggacgtc

SEQ ID NO: 152 - protein, 17 aa, Homo sapiens
ARDQDYYGSGSSYGMDV

SEQ ID NO: 153 - DNA, 324 bp, Homo sapiens
  source 1..324: mol_type = other DNA
gacatccagatgacccagtcgccagcctccctgtctgcatctgtaggagacagagtcaccatcacttgccgggcaagtcagagcattagcagctatttaaattggtatcagcagaaaccagggaaagcccctaagcgcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcacttttggccaggggaccaagctggagatcaaacga

SEQ ID NO: 154 - protein, 108 aa, Homo sapiens
DIQMTQSPASLSASVGDRVTITCRASQSISSYLNWYQQKPGKAPKRLIYAASSLQSGVPSRFSGSGSGTDFTLTISSLQPEDFATYYCQQSYSTPLTFGQGTKLEIKR

SEQ ID NO: 155 - DNA, 18 bp, Homo sapiens
  source 1..18: mol_type = other DNA
cagagcattagcagctat

SEQ ID NO: 156 - protein, 6 aa, Homo sapiens
QSISSY

SEQ ID NO: 157-158 - skipped (2 entries)

SEQ ID NO: 159 - DNA, 27 bp, Homo sapiens
  source 1..27: mol_type = other DNA
caacagagttacagtacccctctcact

SEQ ID NO: 160 - protein, 9 aa, Homo sapiens
QQSYSTPLT

SEQ ID NO: 161 - DNA, 372 bp, Homo sapiens
  source 1..372: mol_type = other DNA
caggtgcagctggtggagtctgggggaggcgtggtccagcctgggaggtccctgagactctcctgtgcagcgtctggattcaccttcagtacctatggcatgcactgggtccgccaggctccaggcaaggggctggagtgggtggcagttatatggtatgatggaactaataaatactatgcagactccgtgaagggccgattcaccatctccagagacaattccaagaacacgctgtatctgcaaatgatcagcctgagagccgaggacacggctgtgtattactgtgcgagagacccctcaggtggggaccactactattactacggtatggacgtctggggccaagggaccacggtcaccgtctcctca

SEQ ID NO: 162 - protein, 124 aa, Homo sapiens
QVQLVESGGGVVQPGRSLRLSCAASGFTFSTYGMHWVRQAPGKGLEWVAVIWYDGTNKYYADSVKGRFTISRDNSKNTLYLQMISLRAEDTAVYYCARDPSGGDHYYYYGMDVWGQGTTVTVSS

SEQ ID NO: 163 - DNA, 24 bp, Homo sapiens
  source 1..24: mol_type = other DNA
ggattcaccttcagtacctatggc

SEQ ID NO: 164 - protein, 8 aa, Homo sapiens
GFTFSTYG

SEQ ID NO: 165 - DNA, 24 bp, Homo sapiens
  source 1..24: mol_type = other DNA
atatggtatgatggaactaataaa

SEQ ID NO: 166 - protein, 8 aa, Homo sapiens
IWYDGTNK

SEQ ID NO: 167 - DNA, 51 bp, Homo sapiens
  source 1..51: mol_type = other DNA
gcgagagacccctcaggtggggaccactactattactacggtatggacgtc

SEQ ID NO: 168 - protein, 17 aa, Homo sapiens
ARDPSGGDHYYYYGMDV

SEQ ID NO: 169 - DNA, 324 bp, Homo sapiens
  source 1..324: mol_type = other DNA
gacatccagatgacccagtctccatcctccctgtctgcatctgtaggagacagaatcaccatcacttgccaggcgagtcaggacattagcaactatttaaattggtatcagcagaagccagggaaagcccctaacctcctgatctccgatgcatccgatttggaaacaggggtcccatcaaggttcagtggaagtggatctgggacagattttactttcaccatcagcagcctgcagcctgaagattttgcaacatattactgtcaacagtatgataatataccgatcaccttcggccaagggacacgactggagattaaacga

SEQ ID NO: 170 - protein, 108 aa, Homo sapiens
DIQMTQSPSSLSASVGDRITITCQASQDISNYLNWYQQKPGKAPNLLISDASDLETGVPSRFSGSGSGTDFTFTISSLQPEDFATYYCQQYDNIPITFGQGTRLEIKR

SEQ ID NO: 171 - DNA, 18 bp, Homo sapiens
  source 1..18: mol_type = other DNA
caggacattagcaactat

SEQ ID NO: 172 - protein, 6 aa, Homo sapiens
QDISNY

SEQ ID NO: 173-174 - skipped (2 entries)

SEQ ID NO: 175 - DNA, 27 bp, Homo sapiens
  source 1..27: mol_type = other DNA
caacagtatgataatataccgatcacc

SEQ ID NO: 176 - protein, 9 aa, Homo sapiens
QQYDNIPIT

SEQ ID NO: 177 - DNA, 366 bp, Homo sapiens
  source 1..366: mol_type = other DNA
caggtgcagctggtggagtcagggggaggcgtggtccagcctgggaggtccctgagactctcctgtgcagcctctggattcaccttcagtagctatggcatgcactgggtccgccaggctccaggcaaggggctggagtgggtgacatttatatcatttgatgaaaggaataaatactatgcagactccgttaagggccgattcaccatctccagagacaattccaagaacacgctgtatctgcaaatgaacagcctgagagctgaggacacggctatgtattactgtgcgagcgaagtcgggtacagttttggtcatgatgcttttgatatctggggccaagggacaatggtcaccgtctcttca

SEQ ID NO: 178 - protein, 122 aa, Homo sapiens
QVQLVESGGGVVQPGRSLRLSCAASGFTFSSYGMHWVRQAPGKGLEWVTFISFDERNKYYADSVKGRFTISRDNSKNTLYLQMNSLRAEDTAMYYCASEVGYSFGHDAFDIWGQGTMVTVSS

SEQ ID NO: 179 - DNA, 24 bp, Homo sapiens
  source 1..24: mol_type = other DNA
ggattcaccttcagtagctatggc

SEQ ID NO: 180 - protein, 8 aa, Homo sapiens
GFTFSSYG

SEQ ID NO: 181 - DNA, 24 bp, Homo sapiens
  source 1..24: mol_type = other DNA
atatcatttgatgaaaggaataaa

SEQ ID NO: 182 - protein, 8 aa, Homo sapiens
ISFDERNK

SEQ ID NO: 183 - DNA, 45 bp, Homo sapiens
  source 1..45: mol_type = other DNA
gcgagcgaagtcgggtacagttttggtcatgatgcttttgatatc

SEQ ID NO: 184 - protein, 15 aa, Homo sapiens
ASEVGYSFGHDAFDI

SEQ ID NO: 185 - DNA, 324 bp, Homo sapiens
  source 1..324: mol_type = other DNA
gacatccagatgacccagtctccatcctccctgtctgcatctgtaggagacagagtcaccatcacttgccaggcgagtcaggacattagcaactatttaaattggtatcagaagaaaccagggaaagcccctaaactcctgatctacgatgcatccaatttggaaacaggggtcccgtcaaggttcagtggaagtggatctgggacagattttactttcaccatcagcagcctgcagcctgaagatattgcaacatattactgtcaacagtatgataatttcccgctcactttcggcggagggaccaaggtggagatcaaacga

SEQ ID NO: 186 - protein, 108 aa, Homo sapiens
DIQMTQSPSSLSASVGDRVTITCQASQDISNYLNWYQKKPGKAPKLLIYDASNLETGVPSRFSGSGSGTDFTFTISSLQPEDIATYYCQQYDNFPLTFGGGTKVEIKR

SEQ ID NO: 187 - DNA, 18 bp, Homo sapiens
  source 1..18: mol_type = other DNA
caggacattagcaactat

SEQ ID NO: 188 - protein, 6 aa, Homo sapiens
QDISNY

SEQ ID NO: 189-190 - skipped (2 entries)

SEQ ID NO: 191 - DNA, 27 bp, Homo sapiens
  source 1..27: mol_type = other DNA
caacagtatgataatttcccgctcact

SEQ ID NO: 192 - protein, 9 aa, Homo sapiens
QQYDNFPLT

SEQ ID NO: 193 - DNA, 369 bp, Homo sapiens
  source 1..369: mol_type = other DNA
gaggtgcagctggtggagtctgggggaggcttggtacagcctggggggtccctgagactctcctgtgcagcctctggattcacctttaacaactatgccatgagctgggtccgccaggctccagggagggggctggagtgggtctcagctattagtggtagtggtgatagcacatactactcagactccgtgaagggccggttcaccatctccagagacaattccaagaacacgctgtatctgcaaatgaacagcctgagagccgaggacacggccgtatattactgtgcgaaagatcagggcctgtattactatggttcggggagttttgactactggggccagggaaccctggtcaccgtctcctca

SEQ ID NO: 194 - protein, 123 aa, Homo sapiens
EVQLVESGGGLVQPGGSLRLSCAASGFTFNNYAMSWVRQAPGRGLEWVSAISGSGDSTYYSDSVKGRFTISRDNSKNTLYLQMNSLRAEDTAVYYCAKDQGLYYYGSGSFDYWGQGTLVTVSS

SEQ ID NO: 195 - DNA, 24 bp, Homo sapiens
  source 1..24: mol_type = other DNA
ggattcacctttaacaactatgcc

SEQ ID NO: 196 - protein, 8 aa, Homo sapiens
GFTFNNYA

SEQ ID NO: 197 - DNA, 24 bp, Homo sapiens
  source 1..24: mol_type = other DNA
attagtggtagtggtgatagcaca

SEQ ID NO: 198 - protein, 8 aa, Homo sapiens
ISGSGDST

SEQ ID NO: 199 - DNA, 48 bp, Homo sapiens
  source 1..48: mol_type = other DNA
gcgaaagatcagggcctgtattactatggttcggggagttttgactac

SEQ ID NO: 200 - protein, 16 aa, Homo sapiens
AKDQGLYYYGSGSFDY

SEQ ID NO: 201 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatccaagctgcatccagtttgcaaagtggggtcccatcaaggttcagtggcagtggatctgggacagatatcactctcaccatcagcagtctgcaacccgaagattttgcaacttactactgtcaacagagttacagtaccccattcactttcggccctgggaccaaagtggatatcaaacga

SEQ ID NO: 202 - protein, 108 aa, Homo sapiens
DIQMTQSPSSLSASVGDRVTITCRASQSISSYLNWYQQKPGKAPKLLIQAASSLQSGVPSRFSGSGSGTDITLTISSLQPEDFATYYCQQSYSTPFTFGPGTKVDIKR

SEQ ID NO: 203 - DNA, 18 bp, Homo sapiens
  source 1..18: mol_type = other DNA
cagagcattagcagctat

SEQ ID NO: 204 - protein, 6 aa, Homo sapiens
QSISSY

SEQ ID NO: 205-206 - skipped (2 entries)

SEQ ID NO: 207 - DNA, 27 bp, Homo sapiens
  source 1..27: mol_type = other DNA
caacagagttacagtaccccattcact

SEQ ID NO: 208 - protein, 9 aa, Homo sapiens
QQSYSTPFT

SEQ ID NO: 209 - DNA, 375 bp, Homo sapiens
  source 1..375: mol_type = other DNA
gaggtgcagctggtggagtctgggggaggcttggtccagcctggggggtccctgcaactctcctgtgcagcctctgggtttgccttcagcgactctgctatatactgggtccgccaggcttccgggaaagggctggagtgggttggccgcattagaaacaaagctaataggttcgcgacagcatatggtgcgtcggtgaaaggcaggttcagcatacacagagatgattcaaagaacacggcgtatctacaaatgaacagcctgaaaaccgaggacacggccgtgtattactgtgccagacatggacacgatactttgactgagggctacggtatggacgtctggggccaagggaccacggtcaccgtctcctca

SEQ ID NO: 210 - protein, 125 aa, Homo sapiens
EVQLVESGGGLVQPGGSLQLSCAASGFAFSDSAIYWVRQASGKGLEWVGRIRNKANRFATAYGASVKGRFSIHRDDSKNTAYLQMNSLKTEDTAVYYCARHGHDTLTEGYGMDVWGQGTTVTVSS

SEQ ID NO: 211 - DNA, 24 bp, Homo sapiens
  source 1..24: mol_type = other DNA
gggtttgccttcagcgactctgct

SEQ ID NO: 212 - protein, 8 aa, Homo sapiens
GFAFSDSA

SEQ ID NO: 213 - DNA, 30 bp, Homo sapiens
  source 1..30: mol_type = other DNA
attagaaacaaagctaataggttcgcgaca

SEQ ID NO: 214 - protein, 10 aa, Homo sapiens
IRNKANRFAT

SEQ ID NO: 215 - DNA, 48 bp, Homo sapiens
  source 1..48: mol_type = other DNA
gccagacatggacacgatactttgactgagggctacggtatggacgtc

SEQ ID NO: 216 - protein, 16 aa, Homo sapiens
ARHGHDTLTEGYGMDV

SEQ ID NO: 217 - DNA, 324 bp, Homo sapiens
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 218 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 219 - DNA, 18 bp, Homo sapiens
  source 1..18: mol_type = other DNA
cagagcattagcagctat

SEQ ID NO: 220 - protein, 6 aa, Homo sapiens
QSISSY

SEQ ID NO: 221-222 - skipped (2 entries)

SEQ ID NO: 223 - DNA, 30 bp, Homo sapiens
  source 1..30: mol_type = other DNA
caacagagttacagtacccctccgatcacc

SEQ ID NO: 224 - protein, 10 aa, Homo sapiens
QQSYSTPPIT

SEQ ID NO: 225 - DNA, 363 bp, Homo sapiens
  source 1..363: mol_type = other DNA
caggtgcagctggtgcagtctggggctgaggtaaagaaccctgggtcctcggtgaaggtctcctgcaaggcttctggaggcaccttcagcagttatactatcaactgggtgcgacaggcccctggacaagggcttgagtggatgggagggatcatccctctctatggaacagcaaactacgcacagaagttccaggccagagtcacgatttccacggacgaatccacgaacacagcctacatggaactgagcaacctgagatttgaagacacggccgtgtatttctgtgcgagtacactggaactacgggcttttgatgcctttgatatctggggccaagggacaatggtcaccgtctcttca

SEQ ID NO: 226 - protein, 121 aa, Homo sapiens
QVQLVQSGAEVKNPGSSVKVSCKASGGTFSSYTINWVRQAPGQGLEWMGGIIPLYGTANYAQKFQARVTISTDESTNTAYMELSNLRFEDTAVYFCASTLELRAFDAFDIWGQGTMVTVSS

SEQ ID NO: 227 - DNA, 24 bp, Homo sapiens
  source 1..24: mol_type = other DNA
ggaggcaccttcagcagttatact

SEQ ID NO: 228 - protein, 8 aa, Homo sapiens
GGTFSSYT

SEQ ID NO: 229 - DNA, 24 bp, Homo sapiens
  source 1..24: mol_type = other DNA
atcatccctctctatggaacagca

SEQ ID NO: 230 - protein, 8 aa, Homo sapiens
IIPLYGTA

SEQ ID NO: 231 - DNA, 42 bp, Homo sapiens
  source 1..42: mol_type = other DNA
gcgagtacactggaactacgggcttttgatgcctttgatatc

SEQ ID NO: 232 - protein, 14 aa, Homo sapiens
ASTLELRAFDAFDI

SEQ ID NO: 233 - DNA, 357 bp, Homo sapiens
  source 1..357: mol_type = other DNA
caggtgcagctgcaggagtcgggcccaggactggtgaagccttcacagaccctgtccctcacctgcactgtctctggtggctccatcagcagtggtggttactactggaactggatccgccagcacccagggaagggcctggagtggattgggtacatctattacagtggaagcacctactacaacccgtccctcaagagtcgagttaccatatcagtagacacgtctaagaaccagttctccctgaagctgggctctgtgactgccgcggacacggccgtgtattactgtgcgcgagctcctccttataactggtttgactactggggccagggaaccctggtcaccgtctcctca

SEQ ID NO: 234 - protein, 119 aa, Homo sapiens
QVQLQESGPGLVKPSQTLSLTCTVSGGSISSGGYYWNWIRQHPGKGLEWIGYIYYSGSTYYNPSLKSRVTISVDTSKNQFSLKLGSVTAADTAVYYCARAPPYNWFDYWGQGTLVTVSS

SEQ ID NO: 235 - DNA, 30 bp, Homo sapiens
  source 1..30: mol_type = other DNA
ggtggctccatcagcagtggtggttactac

SEQ ID NO: 236 - protein, 10 aa, Homo sapiens
GGSISSGGYY

SEQ ID NO: 237 - DNA, 21 bp, Homo sapiens
  source 1..21: mol_type = other DNA
atctattacagtggaagcacc

SEQ ID NO: 238 - protein, 7 aa, Homo sapiens
IYYSGST

SEQ ID NO: 239 - DNA, 33 bp, Homo sapiens
  source 1..33: mol_type = other DNA
gcgcgagctcctccttataactggtttgactac

SEQ ID NO: 240 - protein, 11 aa, Homo sapiens
ARAPPYNWFDY

SEQ ID NO: 241 - DNA, 366 bp, Homo sapiens
  source 1..366: mol_type = other DNA
caggtgcagctggtggagtctgggggaggcttggtcaagcctggagggtccctgagactctcctgtgcagcctctggattcaccttcagtgactactacatgaactgggtccgccaggctccagggaaggggctggaatgggtttcatacattagtaatagtggtaatacccaatactacgcagactctgtgaagggccggttcaccatctccagggacaatgccaagaactccctgtttctgcaaatgaacagcctgcgagccgaggacacggccgtttattactgtacgagagagggactcgaatatagcagctcggagccctttgactactggggccagggaaccctggtcaccgtctcctca

SEQ ID NO: 242 - protein, 122 aa, Homo sapiens
QVQLVESGGGLVKPGGSLRLSCAASGFTFSDYYMNWVRQAPGKGLEWVSYISNSGNTQYYADSVKGRFTISRDNAKNSLFLQMNSLRAEDTAVYYCTREGLEYSSSEPFDYWGQGTLVTVSS

SEQ ID NO: 243 - DNA, 24 bp, Homo sapiens
  source 1..24: mol_type = other DNA
ggattcaccttcagtgactactac

SEQ ID NO: 244 - protein, 8 aa, Homo sapiens
GFTFSDYY

SEQ ID NO: 245 - DNA, 24 bp, Homo sapiens
  source 1..24: mol_type = other DNA
attagtaatagtggtaatacccaa

SEQ ID NO: 246 - protein, 8 aa, Homo sapiens
ISNSGNTQ

SEQ ID NO: 247 - DNA, 45 bp, Homo sapiens
  source 1..45: mol_type = other DNA
acgagagagggactcgaatatagcagctcggagccctttgactac

SEQ ID NO: 248 - protein, 15 aa, Homo sapiens
TREGLEYSSSEPFDY

SEQ ID NO: 249 - DNA, 366 bp, Homo sapiens
  source 1..366: mol_type = other DNA
caggtgcagctggtgcagtctggggctgaagtgaagaagcctggggcctcagtgaaggtctcctgcaagacttctggatacaccttcaccgcctactacatacactgggtgcgacaggcccctggacaagggcttgagtggatgggatggatcaaccctaacaatggtgacacaaactatgcactgaggtttcagggcagggtcaccatgaccagggacatgtccatcaacacagcctacatggagctgcgcgggctgagatctgacgacacggccgtgtattattgtgcgagagatgatctagcagcagcgggtatcggctggttcgactcctggggccagggaaccctggtcaccgtctcctca

SEQ ID NO: 250 - protein, 122 aa, Homo sapiens
QVQLVQSGAEVKKPGASVKVSCKTSGYTFTAYYIHWVRQAPGQGLEWMGWINPNNGDTNYALRFQGRVTMTRDMSINTAYMELRGLRSDDTAVYYCARDDLAAAGIGWFDSWGQGTLVTVSS

SEQ ID NO: 251 - DNA, 24 bp, Homo sapiens
  source 1..24: mol_type = other DNA
ggatacaccttcaccgcctactac

SEQ ID NO: 252 - protein, 8 aa, Homo sapiens
GYTFTAYY

SEQ ID NO: 253 - DNA, 24 bp, Homo sapiens
  source 1..24: mol_type = other DNA
atcaaccctaacaatggtgacaca

SEQ ID NO: 254 - protein, 8 aa, Homo sapiens
INPNNGDT

SEQ ID NO: 255 - DNA, 45 bp, Homo sapiens
  source 1..45: mol_type = other DNA
gcgagagatgatctagcagcagcgggtatcggctggttcgactcc

SEQ ID NO: 256 - protein, 15 aa, Homo sapiens
ARDDLAAAGIGWFDS

SEQ ID NO: 257 - DNA, 366 bp, Homo sapiens
  source 1..366: mol_type = other DNA
gaagtgcagctggtggagtctgggggaggcttgctacagcctggcaggtccctgagactctcctgtgtagcctctggattcacctttgatgattatgccatgcactgggtccggcaagctccagggaagggcctggagtgggtctcaggaattagttggaatagtgaaagtataggctatgcggactctgtgaggggccgattcaccatttccagagacaacgccaagaactccctgtatctccaaatgaacagtctgagagctgaggacacggccttgtattactgtgcaaaagccccgtatagtgggacctacttcgaatacttccgccactggggccagggcaccctggtcaccgtctcctca

SEQ ID NO: 258 - protein, 122 aa, Homo sapiens
EVQLVESGGGLLQPGRSLRLSCVASGFTFDDYAMHWVRQAPGKGLEWVSGISWNSESIGYADSVRGRFTISRDNAKNSLYLQMNSLRAEDTALYYCAKAPYSGTYFEYFRHWGQGTLVTVSS

SEQ ID NO: 259 - DNA, 24 bp, Homo sapiens
  source 1..24: mol_type = other DNA
ggattcacctttgatgattatgcc

SEQ ID NO: 260 - protein, 8 aa, Homo sapiens
GFTFDDYA

SEQ ID NO: 261 - DNA, 24 bp, Homo sapiens
  source 1..24: mol_type = other DNA
attagttggaatagtgaaagtata

SEQ ID NO: 262 - protein, 8 aa, Homo sapiens
ISWNSESI

SEQ ID NO: 263 - DNA, 45 bp, Homo sapiens
  source 1..45: mol_type = other DNA
gcaaaagccccgtatagtgggacctacttcgaatacttccgccac

SEQ ID NO: 264 - protein, 15 aa, Homo sapiens
AKAPYSGTYFEYFRH

SEQ ID NO: 265 - DNA, 357 bp, Homo sapiens
  source 1..357: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatgactggaactacgacgcctttgatatctggggccaagggacaatggtcaccgtctcttca

SEQ ID NO: 266 - protein, 119 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDDWNYDAFDIWGQGTMVTVSS

SEQ ID NO: 267 - DNA, 24 bp, Homo sapiens
  source 1..24: mol_type = other DNA
ggattcaccttcagtagctatggc

SEQ ID NO: 268 - protein, 8 aa, Homo sapiens
GFTFSSYG

SEQ ID NO: 269 - DNA, 24 bp, Homo sapiens
  source 1..24: mol_type = other DNA
atatcatatgatggaagtaataaa

SEQ ID NO: 270 - protein, 8 aa, Homo sapiens
ISYDGSNK

SEQ ID NO: 271 - DNA, 36 bp, Homo sapiens
  source 1..36: mol_type = other DNA
gcgaaagatgactggaactacgacgcctttgatatc

SEQ ID NO: 272 - protein, 12 aa, Homo sapiens
AKDDWNYDAFDI

SEQ ID NO: 273 - DNA, 363 bp, Homo sapiens
  source 1..363: mol_type = other DNA
caggtgcagctgcaggagtcgggcccaggactggtgaagccttcacagaccctgtccctcacctgcactgtctctggtggctccatcagcagtagtggttactactggagctggatccgccagcacccagggaggggcctggagtggattggatacatctattacagtgggagcacctactacaacccgtccctcaagagtcgagttaccatatcagtagacacgtctaagaaccagttctccctgaagctgaactctgtgactgccgcggacacggccgtgtattactgtgcgagagtggactatggttcggggagttcgtttgactactggggccagggaaccctggtcaccgtctcctca

SEQ ID NO: 274 - protein, 121 aa, Homo sapiens
QVQLQESGPGLVKPSQTLSLTCTVSGGSISSSGYYWSWIRQHPGRGLEWIGYIYYSGSTYYNPSLKSRVTISVDTSKNQFSLKLNSVTAADTAVYYCARVDYGSGSSFDYWGQGTLVTVSS

SEQ ID NO: 275 - DNA, 30 bp, Homo sapiens
  source 1..30: mol_type = other DNA
ggtggctccatcagcagtagtggttactac

SEQ ID NO: 276 - protein, 10 aa, Homo sapiens
GGSISSSGYY

SEQ ID NO: 277 - DNA, 21 bp, Homo sapiens
  source 1..21: mol_type = other DNA
atctattacagtgggagcacc

SEQ ID NO: 278 - protein, 7 aa, Homo sapiens
IYYSGST

SEQ ID NO: 279 - DNA, 39 bp, Homo sapiens
  source 1..39: mol_type = other DNA
gcgagagtggactatggttcggggagttcgtttgactac

SEQ ID NO: 280 - protein, 13 aa, Homo sapiens
ARVDYGSGSSFDY

SEQ ID NO: 281 - DNA, 354 bp, Homo sapiens
  source 1..354: mol_type = other DNA
caggttcagctggtgcagtctggacctgaggtgaagaagcctggggcctcagtgaaggtctcctgcaaggcttctggttacacctttaccagctatggcatcagctgggtgcgacaggcccctggacaagggcttgagtggctgggatggatcagcggtttcaatggtagaacagactatacagagaagctccaggacagaatcaccatgaccacagacagatcctcgagcacagcctacatggaactgaggagcctgagatatgacgacacggccgtgtattactgtgcgagagatggactggaaaaacttggtgactactggggccagggaaccctggtcaccgtctcctca

SEQ ID NO: 282 - protein, 118 aa, Homo sapiens
QVQLVQSGPEVKKPGASVKVSCKASGYTFTSYGISWVRQAPGQGLEWLGWISGFNGRTDYTEKLQDRITMTTDRSSSTAYMELRSLRYDDTAVYYCARDGLEKLGDYWGQGTLVTVSS

SEQ ID NO: 283 - DNA, 24 bp, Homo sapiens
  source 1..24: mol_type = other DNA
ggttacacctttaccagctatggc

SEQ ID NO: 284 - protein, 8 aa, Homo sapiens
GYTFTSYG

SEQ ID NO: 285 - DNA, 24 bp, Homo sapiens
  source 1..24: mol_type = other DNA
atcagcggtttcaatggtagaaca

SEQ ID NO: 286 - protein, 8 aa, Homo sapiens
ISGFNGRT

SEQ ID NO: 287 - DNA, 33 bp, Homo sapiens
  source 1..33: mol_type = other DNA
gcgagagatggactggaaaaacttggtgactac

SEQ ID NO: 288 - protein, 11 aa, Homo sapiens
ARDGLEKLGDY

SEQ ID NO: 289 - DNA, 363 bp, Homo sapiens
  source 1..363: mol_type = other DNA
caggtgcagctggtggagtctgggggaggcgtggtccagccggggaggttcctgagactctcctgtgcagcgtctggattcaccttcagtaactctggcatgcactgggtccgccaggctccaggcaaggggctggagtgggtggcaggaatatggcatgatggaagttataaatattatgtagactccgtgaagggccgattcaccatctccagagacaattctaagaacacgctgtttctgcaaatgaacagcctgcgagccgaggacacggctgtatattattgtgcgagagatgattactatgcttcggggaccagcgtggacgtatggggccaagggaccacggtcaccgtctcctca

SEQ ID NO: 290 - protein, 121 aa, Homo sapiens
QVQLVESGGGVVQPGRFLRLSCAASGFTFSNSGMHWVRQAPGKGLEWVAGIWHDGSYKYYVDSVKGRFTISRDNSKNTLFLQMNSLRAEDTAVYYCARDDYYASGTSVDVWGQGTTVTVSS

SEQ ID NO: 291 - DNA, 24 bp, Homo sapiens
  source 1..24: mol_type = other DNA
ggattcaccttcagtaactctggc

SEQ ID NO: 292 - protein, 8 aa, Homo sapiens
GFTFSNSG

SEQ ID NO: 293 - DNA, 24 bp, Homo sapiens
  source 1..24: mol_type = other DNA
atatggcatgatggaagttataaa

SEQ ID NO: 294 - protein, 8 aa, Homo sapiens
IWHDGSYK

SEQ ID NO: 295 - DNA, 42 bp, Homo sapiens
  source 1..42: mol_type = other DNA
gcgagagatgattactatgcttcggggaccagcgtggacgta

SEQ ID NO: 296 - protein, 14 aa, Homo sapiens
ARDDYYASGTSVDV

SEQ ID NO: 297 - DNA, 366 bp, Homo sapiens
  source 1..366: mol_type = other DNA
caggtgcagctggtgcagtctggggctgaggtgaagaagcctggggcctcagtgagggtctcctgcatggcctctggatacaccttcaccggctactatatgcactgggtgcgacaggcccctggacaagggcttgagtggatgggatggatcaaccctaacagtggtggcacaaaatatgcacagaagtttcagggcagggtcaccatgaccagggacacgtccatcagcacagcctacatggagctgagcagactgagatctgacgacacggccgtatattactgtgcgagagaagaagtcgacgatttttggagtggttaccttgactactggggccagggaaccctggtcaccgtctcctca

SEQ ID NO: 298 - protein, 122 aa, Homo sapiens
QVQLVQSGAEVKKPGASVRVSCMASGYTFTGYYMHWVRQAPGQGLEWMGWINPNSGGTKYAQKFQGRVTMTRDTSISTAYMELSRLRSDDTAVYYCAREEVDDFWSGYLDYWGQGTLVTVSS

SEQ ID NO: 299 - DNA, 24 bp, Homo sapiens
  source 1..24: mol_type = other DNA
ggatacaccttcaccggctactat

SEQ ID NO: 300 - protein, 8 aa, Homo sapiens
GYTFTGYY

SEQ ID NO: 301 - DNA, 24 bp, Homo sapiens
  source 1..24: mol_type = other DNA
atcaaccctaacagtggtggcaca

SEQ ID NO: 302 - protein, 8 aa, Homo sapiens
INPNSGGT

SEQ ID NO: 303 - DNA, 45 bp, Homo sapiens
  source 1..45: mol_type = other DNA
gcgagagaagaagtcgacgatttttggagtggttaccttgactac

SEQ ID NO: 304 - protein, 15 aa, Homo sapiens
AREEVDDFWSGYLDY

SEQ ID NO: 305 - DNA, 324 bp, Homo sapiens
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 306 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 307 - DNA, 21 bp, Homo sapiens
  source 1..21: mol_type = other DNA
cagagtgttagcagcagctac

SEQ ID NO: 308 - protein, 7 aa, Homo sapiens
QSVSSSY

SEQ ID NO: 309-310 - skipped (2 entries)

SEQ ID NO: 311 - DNA, 27 bp, Homo sapiens
  source 1..27: mol_type = other DNA
cagcagtatggtagctcaccttggacg

SEQ ID NO: 312 - protein, 9 aa, Homo sapiens
QQYGSSPWT

SEQ ID NO: 313 - DNA, 354 bp, Homo sapiens
  source 1..354: mol_type = other DNA
gaggtgcagctggtggagtctggaggagacttggtccagccgggggggtccctgagactctcctgtgcagcctctgggttcgccgtcaatggcgactattttagttgggtccgccaggctccagggaaggggctggagtggatctcagttatttatagcagtggtaacacatactacgcagactccgtgaagggccgattcaccatctccagacacaattccaagaacacgctgtatcttcaaatgagcagcctaagacctgaggacacggccgtgtattactgtgcgagagacttccctccaatgtctggtgcggactactggggccagggaaccctggtcaccgtctcctca

SEQ ID NO: 314 - protein, 118 aa, Homo sapiens
EVQLVESGGDLVQPGGSLRLSCAASGFAVNGDYFSWVRQAPGKGLEWISVIYSSGNTYYADSVKGRFTISRHNSKNTLYLQMSSLRPEDTAVYYCARDFPPMSGADYWGQGTLVTVSS

SEQ ID NO: 315 - DNA, 24 bp, Homo sapiens
  source 1..24: mol_type = other DNA
gggttcgccgtcaatggcgactat

SEQ ID NO: 316 - protein, 8 aa, Homo sapiens
GFAVNGDY

SEQ ID NO: 317 - DNA, 21 bp, Homo sapiens
  source 1..21: mol_type = other DNA
atttatagcagtggtaacaca

SEQ ID NO: 318 - protein, 7 aa, Homo sapiens
IYSSGNT

SEQ ID NO: 319 - DNA, 36 bp, Homo sapiens
  source 1..36: mol_type = other DNA
gcgagagacttccctccaatgtctggtgcggactac

SEQ ID NO: 320 - protein, 12 aa, Homo sapiens
ARDFPPMSGADY

SEQ ID NO: 321 - DNA, 366 bp, Homo sapiens
  source 1..366: mol_type = other DNA
caggtccagctggtgcagtctggggctgaggtgaagaagcctggggcctcagtgaaggtctcctgcaaggtttccggatacaccctcactgaattgtccatgcactgggtgcgacaggctcctggaaaagggcttgaatggatgggaggttttgatcctgaacatggtaaaataatctacgcacagaaattccagggcagagtcaccatgaccgaggacacatctacagacacagcctacatggaactgagcagcctgagatctgaggacacggccgtctattactgtgcaacattttataactggaactcctactacttcggtatggacgtctggggccacgggaccacggtcaccgtctcctca

SEQ ID NO: 322 - protein, 122 aa, Homo sapiens
QVQLVQSGAEVKKPGASVKVSCKVSGYTLTELSMHWVRQAPGKGLEWMGGFDPEHGKIIYAQKFQGRVTMTEDTSTDTAYMELSSLRSEDTAVYYCATFYNWNSYYFGMDVWGHGTTVTVSS

SEQ ID NO: 323 - DNA, 24 bp, Homo sapiens
  source 1..24: mol_type = other DNA
ggatacaccctcactgaattgtcc

SEQ ID NO: 324 - protein, 8 aa, Homo sapiens
GYTLTELS

SEQ ID NO: 325 - DNA, 24 bp, Homo sapiens
  source 1..24: mol_type = other DNA
tttgatcctgaacatggtaaaata

SEQ ID NO: 326 - protein, 8 aa, Homo sapiens
FDPEHGKI

SEQ ID NO: 327 - DNA, 45 bp, Homo sapiens
  source 1..45: mol_type = other DNA
gcaacattttataactggaactcctactacttcggtatggacgtc

SEQ ID NO: 328 - protein, 15 aa, Homo sapiens
ATFYNWNSYYFGMDV

SEQ ID NO: 329 - DNA, 375 bp, Homo sapiens
  source 1..375: mol_type = other DNA
gaggtgcagctggtggagtcggggggaggcttggtacagcctggggggtccctgagactctcctgtgcagcctctggattcacctttagcagctatgccatgaactgggtccgccaggctccaggaaagggactggagtgggtctcagctgttagtggaagtgctgatatcacaaactacgcagactccgtgaagggccggttcaccatctccagagacaattccaaacacacgctgtatctgcaaatgaacagcctgagagccgaggacacggccgtatattactgtgcgaaggataaagtgtataactggaactacgggatctactacggtatggacgtctggggccaagggaccacggtcaccgtctcctca

SEQ ID NO: 330 - protein, 125 aa, Homo sapiens
EVQLVESGGGLVQPGGSLRLSCAASGFTFSSYAMNWVRQAPGKGLEWVSAVSGSADITNYADSVKGRFTISRDNSKHTLYLQMNSLRAEDTAVYYCAKDKVYNWNYGIYYGMDVWGQGTTVTVSS

SEQ ID NO: 331 - DNA, 24 bp, Homo sapiens
  source 1..24: mol_type = other DNA
ggattcacctttagcagctatgcc

SEQ ID NO: 332 - protein, 8 aa, Homo sapiens
GFTFSSYA

SEQ ID NO: 333 - DNA, 24 bp, Homo sapiens
  source 1..24: mol_type = other DNA
gttagtggaagtgctgatatcaca

SEQ ID NO: 334 - protein, 8 aa, Homo sapiens
VSGSADIT

SEQ ID NO: 335 - DNA, 54 bp, Homo sapiens
  source 1..54: mol_type = other DNA
gcgaaggataaagtgtataactggaactacgggatctactacggtatggacgtc

SEQ ID NO: 336 - protein, 18 aa, Homo sapiens
AKDKVYNWNYGIYYGMDV

SEQ ID NO: 337 - DNA, 363 bp, Homo sapiens
  source 1..363: mol_type = other DNA
cagctgcagctgcaggagtcgggcccaggactggtgaagccttcggagaccctgtccctcacctgcactgtctctggtggctccatcagcagtagtagttactactggggctggatccgccagcccccagggaagggactagagtggattgggagtatctattatagtgggagcacctactacaatccgtccctcaagagtcgagtcaccatatccgtagacacgtccaagaaccagttctccctgaagctgagctctgtgaccgccgcagacacggctgtgtattactgtgcgagacaagggaggtgggagcgagaaaactttgactactggggccagggaaccctggtcaccgtctcctca

SEQ ID NO: 338 - protein, 121 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARQGRWERENFDYWGQGTLVTVSS

SEQ ID NO: 339 - DNA, 30 bp, Homo sapiens; identical to SEQ ID NO: 35
  source 1..30: mol_type = other DNA

SEQ ID NO: 340 - protein, 10 aa, Homo sapiens
GGSISSSSYY

SEQ ID NO: 341 - DNA, 21 bp, Homo sapiens
  source 1..21: mol_type = other DNA
atctattatagtgggagcacc

SEQ ID NO: 342 - protein, 7 aa, Homo sapiens
IYYSGST

SEQ ID NO: 343 - DNA, 39 bp, Homo sapiens
  source 1..39: mol_type = other DNA
gcgagacaagggaggtgggagcgagaaaactttgactac

SEQ ID NO: 344 - protein, 13 aa, Homo sapiens
ARQGRWERENFDY

SEQ ID NO: 345 - DNA, 354 bp, Homo sapiens
  source 1..354: mol_type = other DNA
caggtgcagctacagcagtggggcgcaggactattgaagccttcggagaccctgtccctcacctgcgctgtctctgatgagtccttcagtgattactactggacctggatccgccagcccccagggaaggggctggagtggattggggaaattactcatagtggaagtacccactacaacccgtccctcaagagccgagtcaccctgtcagttgacacgtccaagaaccacttctccctgagcctcaactctgtgaccgccgcggacacggctatttattactgtgcgagaggcggtgactacggtggtttacttgactactggggccagggaaccctggtcaccgtctcctca

SEQ ID NO: 346 - protein, 118 aa, Homo sapiens
QVQLQQWGAGLLKPSETLSLTCAVSDESFSDYYWTWIRQPPGKGLEWIGEITHSGSTHYNPSLKSRVTLSVDTSKNHFSLSLNSVTAADTAIYYCARGGDYGGLLDYWGQGTLVTVSS

SEQ ID NO: 347 - DNA, 24 bp, Homo sapiens
  source 1..24: mol_type = other DNA
gatgagtccttcagtgattactac

SEQ ID NO: 348 - protein, 8 aa, Homo sapiens
DESFSDYY

SEQ ID NO: 349 - DNA, 21 bp, Homo sapiens
  source 1..21: mol_type = other DNA
attactcatagtggaagtacc

SEQ ID NO: 350 - protein, 7 aa, Homo sapiens
ITHSGST

SEQ ID NO: 351 - DNA, 36 bp, Homo sapiens
  source 1..36: mol_type = other DNA
gcgagaggcggtgactacggtggtttacttgactac

SEQ ID NO: 352 - protein, 12 aa, Homo sapiens
ARGGDYGGLLDY

SEQ ID NO: 353 - DNA, 363 bp, Homo sapiens
  source 1..363: mol_type = other DNA
cagctgcagctgcaggagtcgggcccaggactggtgaagccctcggagaccctgtccctcacctgcactgtctctggtggctccatcagcagtaggagtcactactggggctggatccgccagcccccagggaaggggctggagtggattgggagtatctattatagtgggagcacctattacaacccgtccctcaagagtcgagtcaccatatccgtagacacgtccaagaaccagttctccctgaagctgagctctgtgaccgccgcagacacggctgtgtattactgtgcgagacttggctggtacgcagaggaggcttttgaaatctggggtcaagggacaatggtcaccgtctcttca

SEQ ID NO: 354 - protein, 121 aa, Homo sapiens
QLQLQESGPGLVKPSETLSLTCTVSGGSISSRSHYWGWIRQPPGKGLEWIGSIYYSGSTYYNPSLKSRVTISVDTSKNQFSLKLSSVTAADTAVYYCARLGWYAEEAFEIWGQGTMVTVSS

SEQ ID NO: 355 - DNA, 30 bp, Homo sapiens
  source 1..30: mol_type = other DNA
ggtggctccatcagcagtaggagtcactac

SEQ ID NO: 356 - protein, 10 aa, Homo sapiens
GGSISSRSHY

SEQ ID NO: 357 - DNA, 21 bp, Homo sapiens
  source 1..21: mol_type = other DNA
atctattatagtgggagcacc

SEQ ID NO: 358 - protein, 7 aa, Homo sapiens
IYYSGST

SEQ ID NO: 359 - DNA, 39 bp, Homo sapiens
  source 1..39: mol_type = other DNA
gcgagacttggctggtacgcagaggaggcttttgaaatc

SEQ ID NO: 360 - protein, 13 aa, Homo sapiens
ARLGWYAEEAFEI

SEQ ID NO: 361 - protein, 526 aa, Plasmodium falciparum
MIRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 362 - protein, 344 aa, Plasmodium falciparum
KNVNFLQYHFKELSNYNIANSIDILQEKEGHLDFVIIPHYTFLDYYKHLSYNSIYHKSSTYGKYIAVDAFIKKINEAYDKVKSKCNDIKNDLIATIKKLEHPYDINNMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQHHHHHH

SEQ ID NO: 363 - protein, 7 aa, synthetic construct
  REGION 1..7: note = Linker sequence
DIEGRMD

SEQ ID NO: 364 - protein, 5 aa, synthetic construct
  REGION 1..5: note = Linker sequence
GGGGS

SEQ ID NO: 365 - protein, 6 aa, synthetic construct
  REGION 1..6: note = HisX6 tag
HHHHHH

(19) United States
(12) Patent Application Publication
(10) Pub. No.: US 2024/0400663 A1
(43) Pub. Date: Dec. 5, 2024

(54) Anti-PfRH5 Antibodies and Antigen-Binding Fragments Thereof

(71) Applicant: Regeneron Pharmaceuticals, Inc., Tarrytown, NY (US)

(72) Inventor: Lisa Purcell, Yorktown Heights, NY (US)

(21) Appl. No.: 18/807,840

(22) Filed: Aug. 16, 2024

Related U.S. Application Data
(62) Division of application No. 18/139,626, filed on Apr. 26, 2023, now Pat. No. 12,091,449.
Application No. 18/139,626 is a division of application No. 17/041,595, filed on Sep. 25, 2020, now Pat. No. 11,667,701.
(60) Provisional application No. 62/648,259, filed on Mar. 26, 2018.

(51) Int. Cl. C07K 16/20; A61K 9/00; A61K 39/015

(52) U.S. Cl. CPC C07K 16/205; A61K 9/0019; A61K 39/015; C07K 2317/14; C07K 2317/31; C07K 2317/565; C07K 2317/73; C07K 2317/92

(57) ABSTRACT

The present invention includes antibodies and antigen-binding fragments thereof that specifically bind to Plasmodium falciparum reticulocyte binding protein homologue 5 (PfRH5), compositions thereof and methods of making such antibodies, fragments and compositions. Method and compositions for treating, preventing or diagnosing Plasmodium falciparum infection and malaria are also part of the present invention.

### CROSS-REFERENCE TO RELATED APPLICATIONS

This application is a division of U.S. application Ser. No. 18/139,626, filed Apr. 26, 2023, which is a division of U.S. application Ser. No. 17/041,595, filed Sep. 25, 2020, which is a US National Stage Application under 35 USC § 371 of PCT/US2019/023734, filed Mar. 22, 2019, which claims the benefit under 35 USC § 119 (e) of U.S. provisional patent application No. 62/648,259, filed Mar. 26, 2018, each of which is herein incorporated by reference in its entirety.

### REFERENCE TO A SEQUENCE LISTING

This application incorporates by reference a computer readable Sequence Listing in ST.26 XML format, titled 10437US03_Sequence, created on Aug. 16, 2024 and containing 320,268 bytes.

### FIELD OF THE INVENTION

The present invention relates, in part, to antibodies and antigen-binding fragments thereof that bind specifically to PfRH5 as well as methods of use thereof for treating or preventing Plasmodium falciparum infections.

### BACKGROUND OF THE INVENTION

Invasion of host erythrocytes is an essential step of the Plasmodium falciparum life cycle and of malaria pathology. Multiple antimalarial drugs target the asexual blood stages, however, their efficacy is threatened by the appearance of drug resistant strains (Arrow et al., Saving Lives, Buying Time: Economics of Malaria Drugs in an Age of Resistance. National Academies Press (US). 254-266 (2004). PMID: 25009879; and Wright et al., Structure of malaria invasion protein RH5 with erythrocyte basigin and blocking antibodies, Nature: 515: 427-430 (2014). PMID: 25132548). Furthermore, antimalarial drugs display different pharmacokinetic properties. Some antimalarial drugs, such as artemisinin and quinine, are rapidly cleared within one parasite life cycle. On the other hand, hydrophobic and lipophilic antimalarial drugs are eliminated slowly, but they are characterized by different absorption rates depending on the amount of dietary fat consumed (Arrow et al.).

Plasmodium falciparum Reticulocyte Binding Protein Homologue 5 (PfRH5) is a member of the super family of erythrocyte ligands referred to as the Reticulocyte Binding Like proteins (RBLs). PfRH5 binds erythrocytes, is likely essential for blood-stage growth of the parasite and is implicated in the species tropism of erythrocyte invasion. Evidence suggests that a receptor for PfRH5 on erythrocytes is the Ok blood group antigen, basigin (BSG; CD147).

### SUMMARY OF THE INVENTION

The present invention provides an antigen-binding protein (e.g., antibody or antigen-binding fragment thereof) that (i) specifically binds to the same epitope on Plasmodium falciparum Reticulocyte Binding Protein Homologue 5 (PfRH5) as; or (ii) competes for binding to PfRH5 polypeptide with: an antibody or antigen-binding fragment thereof that comprises (a) a heavy chain immunoglobulin that comprises CDR-H1, CDR-H2 and CDR-H3 of a heavy chain immunoglobulin that comprises the amino acid sequence set forth in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354; and/or (b) a light chain immunoglobulin that comprises CDR-L1, CDR-L2 and CDR-L3 of a light chain immunoglobulin that comprises the amino acid sequence set forth in SEQ ID NO: 10, 26, 42, 58, 74, 90, 106, 122, 138, 154, 170, 186, 202, 218 or 306. For example, in an embodiment of the invention, the anti-PfRH5 antigen-binding protein (e.g., antibody or antigen-binding fragment thereof) comprises (i) a heavy chain immunoglobulin that comprises CDR-H1, CDR-H2 and CDR-H3 of a heavy chain immunoglobulin that comprises the amino acid sequence set forth in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354; and/or (ii) a light chain immunoglobulin that comprises CDR-L1, CDR-L2 and CDR-L3 of a light chain immunoglobulin that comprises the amino acid sequence set forth in SEQ ID NO: 10, 26, 42, 58, 74, 90, 106, 122, 138, 154, 170, 186, 202, 218 or 306. For example, in an embodiment of the invention, the antigen-binding protein (e.g., antibody or antigen-binding fragment thereof) comprises: (a) a heavy chain immunoglobulin variable region comprising an amino acid sequence having at least 90% amino acid sequence identity to the amino acid sequence set forth in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354; and/or (b) a light chain immunoglobulin variable region comprising an amino acid sequence having at least 90% amino acid sequence identity to the amino acid sequence set forth in SEQ ID NO: 10, 26, 42, 58, 74, 90, 106, 122, 138, 154, 170, 186, 202, 218 or 306. In an embodiment of the invention, the antigen-binding protein (e.g., antibody or antigen-binding fragment thereof) comprises (a) a heavy chain immunoglobulin comprising the CDR-H1, CDR-H2 and CDR-H3 of a heavy chain immunoglobulin comprising an amino acid sequence set forth in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354 and at least 90% amino acid sequence identity to the amino acid sequence set forth in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354; and/or (b) an light chain immunoglobulin comprising the CDR-L1, CDR-L2 and CDR-L3 of a light chain immunoglobulin comprising an amino acid sequence set forth in SEQ ID NO: 10, 26, 42, 58, 74, 90, 106, 122, 138, 154, 170, 186, 202, 218 or 306 and at least 90% amino acid sequence identity to the amino acid sequence set forth in SEQ ID NO: 10, 26, 42, 58, 74, 90, 106, 122, 138, 154, 170, 186, 202, 218 or 306. The present invention also includes an antigen-binding protein (e.g., antibody or antigen-binding fragment thereof) comprising: a heavy chain immunoglobulin that comprises a CDR-H1 comprising the amino acid sequence: G Y S F T S Y W (SEQ ID NO: 4); a CDR-H2 comprising the amino acid sequence: I Y P G D S D T (SEQ ID NO: 6); and a CDR-H3 comprising the amino acid sequence: A R Q D I T G T T G F D Y (SEQ ID NO: 8); or a CDR-H1 comprising the amino acid sequence: G F T F S S Y A (SEQ ID NO: 20); a CDR-H2 comprising the amino acid sequence: I S Y D G S N K (SEQ ID NO: 22); and a CDR-H3 comprising the amino acid sequence: A K E R L F G V V S Y Y G M D V (SEQ ID NO: 24); or a CDR-H1 comprising the amino acid sequence: G G S I S S S S Y Y (SEQ ID NO: 36); a CDR-H2 comprising the amino acid sequence: I Y Y S G S T (SEQ ID NO: 38); and a CDR-H3 comprising the amino acid sequence: A R Q D R E A L F D Y (SEQ ID NO: 40); or a CDR-H1 comprising the amino acid sequence: G F R F D D Y A (SEQ ID NO: 52); a CDR-H2 comprising the amino acid sequence: I N W N S G G K (SEQ ID NO: 54); and a CDR-H3 comprising the amino acid sequence: A K D R G I A A R L L S R D A F D M (SEQ ID NO: 56); or a CDR-H1 comprising the amino acid sequence: S F T F S S Y G (SEQ ID NO: 68); a CDR-H2 comprising the amino acid sequence: I S Y D G S N K (SEQ ID NO: 70); and a CDR-H3 comprising the amino acid sequence: A R E V R R Y Y Y Y G M D V (SEQ ID NO: 72); or a CDR-H1 comprising the amino acid sequence: G F T F D D Y A (SEQ ID NO: 84); a CDR-H2 comprising the amino acid sequence: I S W N S G D I (SEQ ID NO: 86); and a CDR-H3 comprising the amino acid sequence: A K D T L S G T G T T W Y Y F D Y (SEQ ID NO: 88); or a CDR-H1 comprising the amino acid sequence: G F T F S S Y G (SEQ ID NO: 100); a CDR-H2 comprising the amino acid sequence: I S Y D G S N K (SEQ ID NO: 102); and a CDR-H3 comprising the amino acid sequence: A Q D G S S A I Y Y F Y G M D V (SEQ ID NO: 104); or a CDR-H1 comprising the amino acid sequence: G F T F S S Y G (SEQ ID NO: 116); a CDR-H2 comprising the amino acid sequence: I W Y D G S N K (SEQ ID NO: 118); and a CDR-H3 comprising the amino acid sequence: A R G E H Y Y G S G P F D P (SEQ ID NO: 120); or a CDR-H1 comprising the amino acid sequence: G G S I S S F G Y Y (SEQ ID NO: 132); a CDR-H2 comprising the amino acid sequence: I Y Y S G S I (SEQ ID NO: 134); and a CDR-H3 comprising the amino acid sequence: A R E R D Y G D Y F D Y (SEQ ID NO: 136); or a CDR-H1 comprising the amino acid sequence: G F T F S S Y G (SEQ ID NO: 148); a CDR-H2 comprising the amino acid sequence: I W Y D G S N K (SEQ ID NO: 150); and a CDR-H3 comprising the amino acid sequence: A R D Q D Y Y G S G S S Y G M D V (SEQ ID NO: 152); or a CDR-H1 comprising the amino acid sequence: G F T F S T Y G (SEQ ID NO: 164); a CDR-H2 comprising the amino acid sequence: I W Y D G T N K (SEQ ID NO: 166); and a CDR-H3 comprising the amino acid sequence: A R D P S G G D H Y Y Y Y G M D V (SEQ ID NO: 168); or a CDR-H1 comprising the amino acid sequence: G F T F S S Y G (SEQ ID NO: 180); a CDR-H2 comprising the amino acid sequence: I S F D E R N K (SEQ ID NO: 182); and a CDR-H3 comprising the amino acid sequence: A S E V G Y S F G H D A F D I (SEQ ID NO: 184); or a CDR-H1 comprising the amino acid sequence: G F T F N N Y A (SEQ ID NO: 196); a CDR-H2 comprising the amino acid sequence: I S G S G D S T (SEQ ID NO: 198); and a CDR-H3 comprising the amino acid sequence: A K D Q G L Y Y Y G S G S F D Y (SEQ ID NO: 200); or a CDR-H1 comprising the amino acid sequence: G F A F S D S A (SEQ ID NO: 212); a CDR-H2 comprising the amino acid sequence: I R N K A N R F A T (SEQ ID NO: 214); and a CDR-H3 comprising the amino acid sequence: A R H G H D T L T E G Y G M D V (SEQ ID NO: 216); or a CDR-H1 comprising the amino acid sequence: G G T F S S Y T (SEQ ID NO: 228); a CDR-H2 comprising the amino acid sequence: I I P L Y G T A (SEQ ID NO: 230); and a CDR-H3 comprising the amino acid sequence: A S T L E L R A F D A F D I (SEQ ID NO: 232); or a CDR-H1 comprising the amino acid sequence: G G S I S S G G Y Y (SEQ ID NO: 236); a CDR-H2 comprising the amino acid sequence: I Y Y S G S T (SEQ ID NO: 238); and a CDR-H3 comprising the amino acid sequence: A R A P P Y N W F D Y (SEQ ID NO: 240); or a CDR-H1 comprising the amino acid sequence: G F T F S D Y Y (SEQ ID NO: 244); a CDR-H2 comprising the amino acid sequence: I S N S G N T Q (SEQ ID NO: 246); and a CDR-H3 comprising the amino acid sequence: T R E G L E Y S S S E P F D Y (SEQ ID NO: 248); or a CDR-H1 comprising the amino acid sequence: G Y T F T A Y Y (SEQ ID NO: 252); a CDR-H2 comprising the amino acid sequence: I N P N N G D T (SEQ ID NO: 254); and a CDR-H3 comprising the amino acid sequence: A R D D L A A A G I G W F D S (SEQ ID NO: 256); or a CDR-H1 comprising the amino acid sequence: G F T F D D Y A (SEQ ID NO: 260); a CDR-H2 comprising the amino acid sequence: I S W N S E S I (SEQ ID NO: 262); and a CDR-H3 comprising the amino acid sequence: A K A P Y S G T Y F E Y F R H (SEQ ID NO: 264); or a CDR-H1 comprising the amino acid sequence: G F T F S S Y G (SEQ ID NO: 268); a CDR-H2 comprising the amino acid sequence: I S Y D G S N K (SEQ ID NO: 270); and a CDR-H3 comprising the amino acid sequence: A K D D W N Y D A F D I (SEQ ID NO: 272); or a CDR-H1 comprising the amino acid sequence: G G S I S S S G Y Y (SEQ ID NO: 276); a CDR-H2 comprising the amino acid sequence: I Y Y S G S T (SEQ ID NO: 278); and a CDR-H3 comprising the amino acid sequence: A R V D Y G S G S S F D Y (SEQ ID NO: 280); or a CDR-H1 comprising the amino acid sequence: G Y T F T S Y G (SEQ ID NO: 284); a CDR-H2 comprising the amino acid sequence: I S G F N G R T (SEQ ID NO: 286); and a CDR-H3 comprising the amino acid sequence: A R D G L E K L G D Y (SEQ ID NO: 288); or a CDR-H1 comprising the amino acid sequence: G F T F S N S G (SEQ ID NO: 292); a CDR-H2 comprising the amino acid sequence: I W H D G S Y K (SEQ ID NO: 294); and a CDR-H3 comprising the amino acid sequence: A R D D Y Y A S G T S V D V (SEQ ID NO: 296); or a CDR-H1 comprising the amino acid sequence: G Y T F T G Y Y (SEQ ID NO: 300); a CDR-H2 comprising the amino acid sequence: I N P N S G G T (SEQ ID NO: 302); and a CDR-H3 comprising the amino acid sequence: A R E E V D D F W S G Y L D Y (SEQ ID NO: 304); or a CDR-H1 comprising the amino acid sequence: G F A V N G D Y (SEQ ID NO: 316); a CDR-H2 comprising the amino acid sequence: I Y S S G N T (SEQ ID NO: 318); and a CDR-H3 comprising the amino acid sequence: A R D F P P M S G A D Y (SEQ ID NO: 320); or a CDR-H1 comprising the amino acid sequence: G Y T L T E L S (SEQ ID NO: 324); a CDR-H2 comprising the amino acid sequence: F D P E H G K I (SEQ ID NO: 326); and a CDR-H3 comprising the amino acid sequence: A T F Y N W N S Y Y F G M D V (SEQ ID NO: 328); or a CDR-H1 comprising the amino acid sequence: G F T F S S Y A (SEQ ID NO: 332); a CDR-H2 comprising the amino acid sequence: V S G S A D I T (SEQ ID NO: 334); and a CDR-H3 comprising the amino acid sequence: A K D K V Y N W N Y G I Y Y G M D V (SEQ ID NO: 336); or a CDR-H1 comprising the amino acid sequence: G G S I S S S S Y Y (SEQ ID NO: 340); a CDR-H2 comprising the amino acid sequence: I Y Y S G S T (SEQ ID NO: 342); and a CDR-H3 comprising the amino acid sequence: A R Q GR WERE N F D Y (SEQ ID NO: 344); or a CDR-H1 comprising the amino acid sequence: D E S F S D Y Y (SEQ ID NO: 348); a CDR-H2 comprising the amino acid sequence: I T H S G S T (SEQ ID NO: 350); and a CDR-H3 comprising the amino acid sequence: A R G G D Y G G L L D Y (SEQ ID NO: 352); and/or a light chain immunoglobulin variable region that comprises a CDR-L1 comprising the amino acid sequence: Q S I R N Y (SEQ ID NO: 12); a CDR-L2 comprising the amino acid sequence: A A S (SEQ ID NO: 14); and a CDR-L3 comprising the amino acid sequence: Q Q S Y S T P F T (SEQ ID NO: 16); or a CDR-L1 comprising the amino acid sequence: Q D I N R D (SEQ ID NO: 28); a CDR-L2 comprising the amino acid sequence: D A S (SEQ ID NO: 30); and a CDR-L3 comprising the amino acid sequence: Q Q Y K N L P Y T (SEQ ID NO: 32); or a CDR-L1 comprising the amino acid sequence: Q R I G S S (SEQ ID NO: 44); a CDR-L2 comprising the amino acid sequence: Y A S (SEQ ID NO: 46); and a CDR-L3 comprising the amino acid sequence: H Q S S T L P T (SEQ ID NO: 48); or a CDR-L1 comprising the amino acid sequence: Q D V S S Y (SEQ ID NO: 60); a CDR-L2 comprising the amino acid sequence: A A S (SEQ ID NO: 62); and a CDR-L3 comprising the amino acid sequence: Q H L N T Y P Y T (SEQ ID NO: 64); or a CDR-L1 comprising the amino acid sequence: Q D I S N Y (SEQ ID NO: 76); a CDR-L2 comprising the amino acid sequence: D A S (SEQ ID NO: 78); and a CDR-L3 comprising the amino acid sequence: Q Q Y N N L P L T (SEQ ID NO: 80); or a CDR-L1 comprising the amino acid sequence: Q G I S S Y (SEQ ID NO: 92); a CDR-L2 comprising the amino acid sequence: A A S (SEQ ID NO: 94); and a CDR-L3 comprising the amino acid sequence: Q Q V N S Y P L T (SEQ ID NO: 96); or a CDR-L1 comprising the amino acid sequence: Q D I N N Y (SEQ ID NO: 108); a CDR-L2 comprising the amino acid sequence: A A S (SEQ ID NO: 110); and a CDR-L3 comprising the amino acid sequence: L Q Y N S Y H P T (SEQ ID NO: 112); or a CDR-L1 comprising the amino acid sequence: Q S I S N Y (SEQ ID NO: 124); a CDR-L2 comprising the amino acid sequence: A A S (SEQ ID NO: 126); and a CDR-L3 comprising the amino acid sequence: Q Q S Y S S P L T (SEQ ID NO: 128); or a CDR-L1 comprising the amino acid sequence: Q S V S S N (SEQ ID NO: 140); a CDR-L2 comprising the amino acid sequence: G A S (SEQ ID NO: 142); and a CDR-L3 comprising the amino acid sequence: Q Q Y N N W P L T (SEQ ID NO: 144); or a CDR-L1 comprising the amino acid sequence: Q S I S S Y (SEQ ID NO: 156); a CDR-L2 comprising the amino acid sequence: A A S (SEQ ID NO: 158); and a CDR-L3 comprising the amino acid sequence: Q Q S Y S T P L T (SEQ ID NO: 160); or a CDR-L1 comprising the amino acid sequence: Q D I S N Y (SEQ ID NO: 172); a CDR-L2 comprising the amino acid sequence: D A S (SEQ ID NO: 174); and a CDR-L3 comprising the amino acid sequence: Q Q Y D N I P I T (SEQ ID NO: 176); or a CDR-L1 comprising the amino acid sequence: Q D I S N Y (SEQ ID NO: 188); a CDR-L2 comprising the amino acid sequence: D A S (SEQ ID NO: 190); and a CDR-L3 comprising the amino acid sequence: Q Q Y D N F P L T (SEQ ID NO: 192); or a CDR-L1 comprising the amino acid sequence: Q S I S S Y (SEQ ID NO: 204); a CDR-L2 comprising the amino acid sequence: A A S (SEQ ID NO: 206); and a CDR-L3 comprising the amino acid sequence: Q Q S Y S T P F T (SEQ ID NO: 208); or a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); or a CDR-L1 comprising the amino acid sequence: Q S V S S S Y (SEQ ID NO: 308); a CDR-L2 comprising the amino acid sequence: G A S (SEQ ID NO: 310); and a CDR-L3 comprising the amino acid sequence: Q Q Y G S S P W T (SEQ ID NO: 312). In an embodiment of the invention, the antigen-binding protein (e.g., antibody or antigen-binding fragment thereof) comprises (1) a heavy chain immunoglobulin variable region that comprises a CDR-H1 comprising the amino acid sequence: G Y S F T S Y W (SEQ ID NO: 4); a CDR-H2 comprising the amino acid sequence: I Y P G D S D T (SEQ ID NO: 6); and a CDR-H3 comprising the amino acid sequence: A R Q D I T G T T G F D Y (SEQ ID NO: 8); and a light chain immunoglobulin variable region that comprises a CDR-L1 comprising the amino acid sequence: Q S I R N Y (SEQ ID NO: 12); a CDR-L2 comprising the amino acid sequence: A A S (SEQ ID NO: 14); and a CDR-L3 comprising the amino acid sequence: Q Q S Y S T P F T (SEQ ID NO: 16); (2) a heavy chain immunoglobulin variable region that comprises: a CDR-H1 comprising the amino acid sequence: G F T F S S Y A (SEQ ID NO: 20); a CDR-H2 comprising the amino acid sequence: I S Y D G S N K (SEQ ID NO: 22); a CDR-H3 comprising the amino acid sequence: A K E R L F G V V S Y Y G M D V (SEQ ID NO: 24); and a light chain immunoglobulin variable region that comprises: a CDR-L1 comprising the amino acid sequence: Q D I N R D (SEQ ID NO: 28); a CDR-L2 comprising the amino acid sequence: D A S (SEQ ID NO: 30); and a CDR-L3 comprising the amino acid sequence: Q Q Y K N L P Y T (SEQ ID NO: 32); (3) a heavy chain immunoglobulin variable region that comprises: a CDR-H1 comprising the amino acid sequence: G G S I S S S S Y Y (SEQ ID NO: 36); a CDR-H2 comprising the amino acid sequence: I Y Y S G S T (SEQ ID NO: 38); and a CDR-H3 comprising the amino acid sequence: A R Q D R E A L F D Y (SEQ ID NO: 40); and a light chain immunoglobulin variable region that comprises: a CDR-L1 comprising the amino acid sequence: Q R I G S S (SEQ ID NO: 44); a CDR-L2 comprising the amino acid sequence: Y A S (SEQ ID NO: 46); and a CDR-L3 comprising the amino acid sequence: H Q S S T L P T (SEQ ID NO: 48); (4) a heavy chain immunoglobulin variable region that comprises: a CDR-H1 comprising the amino acid sequence: G F R F D D Y A (SEQ ID NO: 52); a CDR-H2 comprising the amino acid sequence: I N W N S G G K (SEQ ID NO: 54); and a CDR-H3 comprising the amino acid sequence: A K D R G I A A R L L S R D A F D M (SEQ ID NO: 56); and a light chain immunoglobulin variable region that comprises: a CDR-L1 comprising the amino acid sequence: Q D V S S Y (SEQ ID NO: 60); a CDR-L2 comprising the amino acid sequence: A A S (SEQ ID NO: 62); and a CDR-L3 comprising the amino acid sequence: Q H L N T Y P Y T (SEQ ID NO: 64); (5) a heavy chain immunoglobulin variable region that comprises: a CDR-H1 comprising the amino acid sequence: S F T F S S Y G (SEQ ID NO: 68); a CDR-H2 comprising the amino acid sequence: I S Y D G S N K (SEQ ID NO: 70); and a CDR-H3 comprising the amino acid sequence: A R E V R R Y Y Y Y G M D V (SEQ ID NO: 72); and a light chain immunoglobulin variable region that comprises: a CDR-L1 comprising the amino acid sequence: Q D I S N Y (SEQ ID NO: 76); a CDR-L2 comprising the amino acid sequence: D A S (SEQ ID NO: 78); and a CDR-L3 comprising the amino acid sequence: Q Q Y N N L P L T (SEQ ID NO: 80); (6) a heavy chain immunoglobulin variable region that comprises: a CDR-H1 comprising the amino acid sequence: G F T F D D Y A (SEQ ID NO: 84); a CDR-H2 comprising the amino acid sequence: I S W N S G D I (SEQ ID NO: 86); and a CDR-H3 comprising the amino acid sequence: A K D T L S G T G T T W Y Y F D Y (SEQ ID NO: 88); and a light chain immunoglobulin variable region that comprises: a CDR-L1 comprising the amino acid sequence: Q G I S S Y (SEQ ID NO: 92); a CDR-L2 comprising the amino acid sequence: A A S (SEQ ID NO: 94); and a CDR-L3 comprising the amino acid sequence: Q Q V N S Y P L T (SEQ ID NO: 96); (7) a heavy chain immunoglobulin variable region that comprises: a CDR-H1 comprising the amino acid sequence: G F T F S S Y G (SEQ ID NO: 100); a CDR-H2 comprising the amino acid sequence: I S Y D G S N K (SEQ ID NO: 102); and a CDR-H3 comprising the amino acid sequence: A Q D G S S A I Y Y F Y G M D V (SEQ ID NO: 104); and a light chain immunoglobulin variable region that comprises: a CDR-L1 comprising the amino acid sequence: Q D I N N Y (SEQ ID NO: 108); a CDR-L2 comprising the amino acid sequence: A A S (SEQ ID NO: 110); and a CDR-L3 comprising the amino acid sequence: L Q Y N S Y H P T (SEQ ID NO: 112); (8) a heavy chain immunoglobulin variable region that comprises: a CDR-H1 comprising the amino acid sequence: G F T F S S Y G (SEQ ID NO: 116); a CDR-H2 comprising the amino acid sequence: I W Y D G S N K (SEQ ID NO: 118); and a CDR-H3 comprising the amino acid sequence: A R G E H Y Y G S G P F D P (SEQ ID NO: 120); and a light chain immunoglobulin variable region that comprises: a CDR-L1 comprising the amino acid sequence: Q S I S N Y (SEQ ID NO: 124); a CDR-L2 comprising the amino acid sequence: A A S (SEQ ID NO: 126); and a CDR-L3 comprising the amino acid sequence: Q Q S Y S S P L T (SEQ ID NO: 128); (9) a heavy chain immunoglobulin variable region that comprises: a CDR-H1 comprising the amino acid sequence: G G S I S S F G Y Y (SEQ ID NO: 132); a CDR-H2 comprising the amino acid sequence: I Y Y S G S I (SEQ ID NO: 134); and a CDR-H3 comprising the amino acid sequence: A R E R D Y G D Y F D Y (SEQ ID NO: 136); and a light chain immunoglobulin variable region that comprises: a CDR-L1 comprising the amino acid sequence: Q S V S S N (SEQ ID NO: 140); a CDR-L2 comprising the amino acid sequence: G A S (SEQ ID NO: 142); and a CDR-L3 comprising the amino acid sequence: Q Q Y N N W P L T (SEQ ID NO: 144); (10) a heavy chain immunoglobulin variable region that comprises: a CDR-H1 comprising the amino acid sequence: G F T F S S Y G (SEQ ID NO: 148); a CDR-H2 comprising the amino acid sequence: I W Y D G S N K (SEQ ID NO: 150); and a CDR-H3 comprising the amino acid sequence: A R D Q D Y Y G S G S S Y G M D V (SEQ ID NO: 152); and a light chain immunoglobulin variable region that comprises: a CDR-L1 comprising the amino acid sequence: Q S I S S Y (SEQ ID NO: 156); a CDR-L2 comprising the amino acid sequence: A A S (SEQ ID NO: 158); and a CDR-L3 comprising the amino acid sequence: Q Q S Y S T P L T (SEQ ID NO: 160); (11) a heavy chain immunoglobulin variable region that comprises: a CDR-H1 comprising the amino acid sequence: G F T F S T Y G (SEQ ID NO: 164); a CDR-H2 comprising the amino acid sequence: I W Y D G T N K (SEQ ID NO: 166); and a CDR-H3 comprising the amino acid sequence: A R D P S G G D H Y Y Y Y G M D V (SEQ ID NO: 168); and a light chain immunoglobulin variable region that comprises: a CDR-L1 comprising the amino acid sequence: Q D I S N Y (SEQ ID NO: 172); a CDR-L2 comprising the amino acid sequence: D A S (SEQ ID NO: 174); and a CDR-L3 comprising the amino acid sequence: Q Q Y D N I P I T (SEQ ID NO: 176); (12) a heavy chain immunoglobulin variable region that comprises: a CDR-H1 comprising the amino acid sequence: G F T F S S Y G (SEQ ID NO: 180); a CDR-H2 comprising the amino acid sequence: I S F D E R N K (SEQ ID NO: 182); and a CDR-H3 comprising the amino acid sequence: A S E V G Y S F G H D A F D I (SEQ ID NO: 184); and a light chain immunoglobulin variable region that comprises: a CDR-L1 comprising the amino acid sequence: Q D I S N Y (SEQ ID NO: 188); a CDR-L2 comprising the amino acid sequence: D A S (SEQ ID NO: 190); and a CDR-L3 comprising the amino acid sequence: Q Q Y D N F P L T (SEQ ID NO: 192); (13) a heavy chain immunoglobulin variable region that comprises: a CDR-H1 comprising the amino acid sequence: G F T F N N Y A (SEQ ID NO: 196); a CDR-H2 comprising the amino acid sequence: I S G S G D S T (SEQ ID NO: 198); and a CDR-H3 comprising the amino acid sequence: A K D Q G L Y Y Y G S G S F D Y (SEQ ID NO: 200); and a light chain immunoglobulin variable region that comprises: a CDR-L1 comprising the amino acid sequence: Q S I S S Y (SEQ ID NO: 204); a CDR-L2 comprising the amino acid sequence: A A S (SEQ ID NO: 206); and a CDR-L3 comprising the amino acid sequence: Q Q S Y S T P F T (SEQ ID NO: 208); (14) a heavy chain immunoglobulin variable region that comprises: a CDR-H1 comprising the amino acid sequence: G F A F S D S A (SEQ ID NO: 212); a CDR-H2 comprising the amino acid sequence: I R N K A N R F A T (SEQ ID NO: 214); and a CDR-H3 comprising the amino acid sequence: A R H G H D T L T E G Y G M D V (SEQ ID NO: 216); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (15) a heavy chain immunoglobulin variable region that comprises: a CDR-H1 comprising the amino acid sequence: G G T F S S Y T (SEQ ID NO: 228); a CDR-H2 comprising the amino acid sequence: I I P L Y G T A (SEQ ID NO: 230); and a CDR-H3 comprising the amino acid sequence: A S T L E L R A F D A F D I (SEQ ID NO: 232); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (16) a heavy chain immunoglobulin variable region that comprises: a CDR-H1 comprising the amino acid sequence: G G S I S S G G Y Y (SEQ ID NO: 236); a CDR-H2 comprising the amino acid sequence: I Y Y S G S T (SEQ ID NO: 238); and a CDR-H3 comprising the amino acid sequence: A R A P P Y N W F D Y (SEQ ID NO: 240); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (17) a heavy chain immunoglobulin variable region that comprises: a CDR-H1 comprising the amino acid sequence: G F T F S D Y Y (SEQ ID NO: 244); a CDR-H2 comprising the amino acid sequence: I S N S G N T Q (SEQ ID NO: 246); and a CDR-H3 comprising the amino acid sequence: T R E G L E Y S S S E P F D Y (SEQ ID NO: 248); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (18) a heavy chain immunoglobulin variable region that comprises: a CDR-H1 comprising the amino acid sequence: G Y T F T A Y Y (SEQ ID NO: 252); a CDR-H2 comprising the amino acid sequence: I N P N N G D T (SEQ ID NO: 254); and a CDR-H3 comprising the amino acid sequence: A R D D L A A A G I G W F D S (SEQ ID NO: 256); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (19) a heavy chain immunoglobulin variable region that comprises: a CDR-H1 comprising the amino acid sequence: G F T F D D Y A (SEQ ID NO: 260); a CDR-H2 comprising the amino acid sequence: I S W N S E S I (SEQ ID NO: 262); and a CDR-H3 comprising the amino acid sequence: A K A P Y S G T Y F E Y F R H (SEQ ID NO: 264); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (20) a heavy chain immunoglobulin variable region that comprises: a CDR-H1 comprising the amino acid sequence: G F T F S S Y G (SEQ ID NO: 268); a CDR-H2 comprising the amino acid sequence: I S Y D G S N K (SEQ ID NO: 270); and a CDR-H3 comprising the amino acid sequence: A K D D W N Y D A F D I (SEQ ID NO: 272); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (21) a heavy chain immunoglobulin variable region that comprises: a CDR-H1 comprising the amino acid sequence: G G S I S S S G Y Y (SEQ ID NO: 276); a CDR-H2 comprising the amino acid sequence: I Y Y S G S T (SEQ ID NO: 278); and a CDR-H3 comprising the amino acid sequence: A R V D Y G S G S S F D Y (SEQ ID NO: 280); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (22) a heavy chain immunoglobulin variable region that comprises: a CDR-H1 comprising the amino acid sequence: G Y T F T S Y G (SEQ ID NO: 284); a CDR-H2 comprising the amino acid sequence: I S G F N G R T (SEQ ID NO: 286); and a CDR-H3 comprising the amino acid sequence: A R D G L E K L G D Y (SEQ ID NO: 288); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (23) a heavy chain immunoglobulin variable region that comprises: a CDR-H1 comprising the amino acid sequence: G F T F S N S G (SEQ ID NO: 292); a CDR-H2 comprising the amino acid sequence: I W H D G S Y K (SEQ ID NO: 294); and a CDR-H3 comprising the amino acid sequence: A R D D Y Y A S G T S V D V (SEQ ID NO: 296); and a light chain immunoglobulin variable region that comprises: a CDR-L1 comprising the amino acid sequence: Q S I S S Y (SEQ ID NO: 220); a CDR-L2 comprising the amino acid sequence: A A S (SEQ ID NO: 222); and a CDR-L3 comprising the amino acid sequence: Q Q S Y S T P P I T (SEQ ID NO: 224); (24) a heavy chain immunoglobulin variable region that comprises: a CDR-H1 comprising the amino acid sequence: G Y T F T G Y Y (SEQ ID NO: 300); a CDR-H2 comprising the amino acid sequence: I N P N S G G T (SEQ ID NO: 302); and a CDR-H3 comprising the amino acid sequence: A R E E V D D F W S G Y L D Y (SEQ ID NO: 304); and a light chain immunoglobulin variable region that comprises: a CDR-L1 comprising the amino acid sequence: Q S V S S S Y (SEQ ID NO: 308); a CDR-L2 comprising the amino acid sequence: G A S (SEQ ID NO: 310); and a CDR-L3 comprising the amino acid sequence: Q Q Y G S S P W T (SEQ ID NO: 312); (25) a heavy chain immunoglobulin variable region that comprises: a CDR-H1 comprising the amino acid sequence: G F A V N G D Y (SEQ ID NO: 316); a CDR-H2 comprising the amino acid sequence: I Y S S G N T (SEQ ID NO: 318); and a CDR-H3 comprising the amino acid sequence: A R D F P P M S G A D Y (SEQ ID NO: 320); and a light chain immunoglobulin variable region that comprises: a CDR-L1 comprising the amino acid sequence: Q S V S S S Y (SEQ ID NO: 308); a CDR-L2 comprising the amino acid sequence: G A S (SEQ ID NO: 310); and a CDR-L3 comprising the amino acid sequence: Q Q Y G S S P W T (SEQ ID NO: 312); (26) a heavy chain immunoglobulin variable region that comprises: a CDR-H1 comprising the amino acid sequence: G Y T L T E L S (SEQ ID NO: 324); a CDR-H2 comprising the amino acid sequence: F D P E H G K I (SEQ ID NO: 326); and a CDR-H3 comprising the amino acid sequence: A T F Y N W N S Y Y F G M D V (SEQ ID NO: 328); and a light chain immunoglobulin variable region that comprises: a CDR-L1 comprising the amino acid sequence: Q S V S S S Y (SEQ ID NO: 308); a CDR-L2 comprising the amino acid sequence: G A S (SEQ ID NO: 310); and a CDR-L3 comprising the amino acid sequence: Q Q Y G S S P W T (SEQ ID NO: 312); (27) a heavy chain immunoglobulin variable region that comprises: a CDR-H1 comprising the amino acid sequence: G F T F S S Y A (SEQ ID NO: 332); a CDR-H2 comprising the amino acid sequence: V S G S A D I T (SEQ ID NO: 334); and a CDR-H3 comprising the amino acid sequence: A K D K V Y N W N Y G I Y Y G M D V (SEQ ID NO: 336); and a light chain immunoglobulin variable region that comprises: a CDR-L1 comprising the amino acid sequence: Q S V S S S Y (SEQ ID NO: 308); a CDR-L2 comprising the amino acid sequence: G A S (SEQ ID NO: 310); and a CDR-L3 comprising the amino acid sequence: Q Q Y G S S P W T (SEQ ID NO: 312); (28) a heavy chain immunoglobulin variable region that comprises: a CDR-H1 comprising the amino acid sequence: G G S I S S S S Y Y (SEQ ID NO: 340); a CDR-H2 comprising the amino acid sequence: I Y Y S G S T (SEQ ID NO: 342); and a CDR-H3 comprising the amino acid sequence: A R Q G R W E R E N F D Y (SEQ ID NO: 344); and a light chain immunoglobulin variable region that comprises: a CDR-L1 comprising the amino acid sequence: Q S V S S S Y (SEQ ID NO: 308); a CDR-L2 comprising the amino acid sequence: G A S (SEQ ID NO: 310); and a CDR-L3 comprising the amino acid sequence: Q Q Y G S S P W T (SEQ ID NO: 312); and/or (29) a heavy chain immunoglobulin variable region that comprises: a CDR-H1 comprising the amino acid sequence: D E S F S D Y Y (SEQ ID NO: 348); a CDR-H2 comprising the amino acid sequence: I T H S G S T (SEQ ID NO: 350); and a CDR-H3 comprising the amino acid sequence: A R G G D Y G G L L D Y (SEQ ID NO: 352); and a light chain immunoglobulin variable region that comprises: a CDR-L1 comprising the amino acid sequence: Q S V S S S Y (SEQ ID NO: 308); a CDR-L2 comprising the amino acid sequence: G A S (SEQ ID NO: 310); and a CDR-L3 comprising the amino acid sequence: Q Q Y G S S P W T (SEQ ID NO: 312). For example, in an embodiment of the invention, the antigen-binding protein (e.g., antibody or antigen-binding fragment thereof) comprises (a) a heavy chain immunoglobulin that comprises the amino acid sequence set forth in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354; and/or (b) a light chain immunoglobulin that comprises the amino acid sequence set forth in SEQ ID NO: 10, 26, 42, 58, 74, 90, 106, 122, 138, 154, 170, 186, 202, 218 or 306. In an embodiment of the invention, the antigen-binding protein is multispecific (e.g., bispecific, multiparatopic or biparatopic).

In an embodiment of the invention, the antigen-binding protein (e.g., antibody or antigen-binding fragment thereof) comprises one or more of the following properties: Inhibits growth of Plasmodium falciparum in human red blood cells; Inhibits growth of Plasmodium falciparum strain D10, Dd2, 7G8, W2-mef, 3D7, HB3, FCR-1/FVO, Cam3.II or RF7 in human red blood cells; Blocks binding of PfRH5 polypeptide to basigin polypeptide; for example, at a concentration of about 6.67 micromolar, causes maximal growth inhibition (e.g., in vitro) of Plasmodium falciparum (e.g., strain FCR-1/FVO) in heat-inactivated human or Aotus monkey serum (e.g., as measured by parasite lactate dehydrogenase (LDH) activity) that at about 1-10% (e.g., 1, 2, 3, 4, 5, 6, 7, 8, 9 or 10%) (relative to uninfected erythrocytes) higher than that of non-heat-inactivated human or Aotus monkey serum, respectively; when exposed to said antigen-binding protein, does not induce mutation of PfRH5 in Plasmodium falciparum (e.g., strain 3D7), e.g., in vitro after about 45 days of gradually increasing antibody concentration, e.g., from about 1XEC50 to about 110XEC50; and/or binds to PfRH5 lacking the amino-terminal residues M1-Y139 and including residues K140-Q526 but lacking K247-L295 and having the mutations T216A and T299A, for example, wherein the antigen-binding protein comprises an amino acid sequence as set forth herein.

The present invention also includes a complex comprising an antigen-binding protein (e.g., antibody or antigen-binding fragment thereof) bound to a Plasmodium Falciparum reticulocyte binding protein homologue 5 (PfRH5) polypeptide. For example, the PfRH5 is on the surface of a cell such as Plasmodium Falciparum (e.g., merozoites of Plasmodium Falciparum), e.g., in the body of a subject (e.g., a human). In an embodiment of the invention, the PfRH5 is on the surface of a Plasmodium Falciparum, e.g., a merozoite in a red blood cell.

The present invention also provides a method for making an anti-PfRH5 antigen-binding protein (e.g., antibody or antigen-binding fragment thereof) set forth herein or immunoglobulin chain thereof comprising: (a) introducing one or more polynucleotides encoding an immunoglobulin chain of said antigen-binding protein into a host cell (e.g., a Chinese hamster ovary cell); (b) culturing the host cell under conditions favorable to expression of the polynucleotide; and (c) optionally, isolating the antigen-binding protein or immunoglobulin chain from the host cell and/or medium in which the host cell is grown. An antigen-binding protein or immunoglobulin chain which is a product of such a method also forms part of the present invention.

A lateral flow immuno-chromatographic antigen-detection test strip comprising an anti-PfRH5 antigen-binding protein set forth herein (e.g., antibody or antigen-binding fragment thereof) is part of the present invention. Methods for detecting Plasmodium falciparum in a blood sample from a subject and/or the body of a subject, using the test strip, are also part of the present invention.

A polypeptide comprising: (a) CDR-H1, CDR-H2, and CDR-H3 of an immunoglobulin heavy chain variable region of an immunoglobulin chain that comprises the amino acid sequence set forth in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354; or (b) CDR-L1, CDR-L2, and CDR-L3 of immunoglobulin light chain variable region of an immunoglobulin chain that comprises the amino acid sequence set forth in SEQ ID NO: 10, 26, 42, 58, 74, 90, 106, 122, 138, 154, 170, 186, 202, 218 or 306 also forms part of the present invention. Polynucleotides (e.g., DNA or RNA) encoding such a polypeptide also form part of the present invention along with a vector that comprises the polynucleotide.

The present invention also provides a host cell (e.g., a Chinese hamster ovary cell) comprising the antigen-binding protein (e.g., antibody or antigen-binding fragment thereof) or an immunoglobulin chain or a polypeptide or polynucleotide or vector which is set forth herein.

The present invention also provides a composition or kit that comprises one or more (e.g., 1, 2, 3 or 4) of the anti-PfRH5 antigen-binding proteins (e.g., antibody or antigen-binding fragment thereof) set forth herein, optionally in association with a further therapeutic agent (e.g., an anti-parasitic drug, chloroquine, atovaquone, proguanil, artemether, lumefantrine, mefloquine, quinine, quinidine, doxycycline (optionally in combination with quinine), clindamycin, a vaccine, an anti-malarial vaccine or RTS,S/AS01). The present invention also provides a pharmaceutical composition comprising an anti-PfRH5 antigen-binding protein set forth herein and pharmaceutically acceptable carrier and, optionally, a further therapeutic agent.

The present invention also provides a vessel or injection device (e.g., an autoinjector or pre-filled syringe) comprising an anti-PfRH5 antigen-binding protein (e.g., antibody or antigen-binding fragment thereof) or composition (e.g., a pharmaceutical composition) set forth herein.

The present invention provides a method for treating or preventing Plasmodium falciparum infection (e.g., malaria) in a subject (e.g., a human) in need thereof, comprising administering (e.g., parenterally) a therapeutically effective amount of an anti-PfFRH5 antigen-binding protein discussed herein optionally in association with a further therapeutic agent. In an embodiment of the invention, the subject is diagnosed as suffering from a Plasmodium falciparum infection (e.g., malaria) prior to initiation of treatment. For example, in an embodiment of the invention, the subject is diagnosed using a lateral flow test strip as set forth herein. In an embodiment of the invention, the subject is not infected with Plasmodium falciparum, but is administered a therapeutically effective amount of the anti-PfRH5 antigen-binding protein prophylactically, i.e., so as to prevent such an infection.

The present invention also provides a method for diagnosing Plasmodium falciparum infection in a subject comprising contacting an anti-PfRH5 antigen-binding protein of the present invention with a sample (e.g., blood) from said subject and, if a complex between the antigen-binding protein and PfRH5 polypeptide in the sample is detected, determining that the subject is infected with Plasmodium falciparum. For example, said complex can be formed on a lateral flow test strip as set forth herein comprising an anti-PfRH5 antigen-binding protein of the present invention and the PfRH5 polypeptide (from the subject's sample).

The present invention provides a method for administering an anti-PfRH5 antigen-binding protein (e.g., antibody or antigen-binding fragment thereof) set forth herein into the body of a subject (e.g., a human) comprising injecting the antigen-binding protein into the body of the subject, optionally in association with a further therapeutic agent, e.g., subcutaneously, intravenously or intramuscularly.

The present invention also encompasses any immunoglobulin polypeptide or polynucleotide set forth herein, e.g., comprising any amino acid sequence set forth in a member selected from the group consisting of SEQ ID NO: 2, 4, 6, 8, 10, 12, 14, 18, 20, 22, 24, 26, 28, 30, 34, 36, 38, 40, 42, 44, 46, 50, 52, 54, 56, 58, 60, 62, 66, 68, 70, 72, 74, 76, 78, 82, 84, 86, 88, 90, 92, 94, 98, 100, 102, 104, 106, 108, 110, 114, 116, 118, 120, 122, 124, 126, 130, 132, 134, 136, 138, 140, 142, 146, 148, 150, 152, 154, 156, 158, 162, 164, 166, 168, 170, 172, 174, 178, 180, 182, 184, 186, 188, 190, 194, 196, 198, 200, 202, 204, 206, 210, 212, 214, 216, 218, 220, 222, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 314, 316, 318, 320, 322, 324, 326, 328, 330, 332, 334, 336, 338, 340, 342, 344, 346, 348, 350, 352, 354, 356, 358 and 360; or a polynucleotide comprising any nucleotide sequence set forth in a member selected from the group consisting of SEQ ID NO: 1, 3, 5, 7, 9, 11, 13, 17, 19, 21, 23, 25, 27, 29, 33, 35, 37, 39, 41, 43, 45, 49, 51, 53, 55, 57, 59, 61, 65, 67, 69, 71, 73, 75, 77, 81, 83, 85, 87, 89, 91, 93, 97, 99, 101, 103, 105, 107, 109, 113, 115, 117, 119, 121, 123, 125, 129, 131, 133, 135, 137, 139, 141, 145, 147, 149, 151, 153, 155, 157, 161, 163, 165, 167, 169, 171, 173, 177, 179, 181, 183, 185, 187, 189, 193, 195, 197, 199, 201, 203, 205, 209, 211, 213, 215, 217, 219, 221, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 313, 315, 317, 319, 321, 323, 325, 327, 329, 331, 333, 335, 337, 339, 341, 343, 345, 347, 349, 351, 353, 355, 357 and 359.

### BRIEF DESCRIPTION OF THE FIGURES

FIG. 1 shows sequence alignments of PfRH5 corresponding to each PfRH5-specific antibody after 45 days of gradual increases in antibody pressure (1×EC₅₀ to 110×EC₅₀).

### DETAILED DESCRIPTION OF THE INVENTION

Plasmodium falciparum is a protozoan parasite, one of the species of Plasmodium that cause malaria in humans which can be transmitted by the female Anopheles mosquito. Malaria caused by this species (which may be referred to as “falciparum malaria”) is a highly dangerous form of malaria, with a high rate of complications and mortality. See e.g., Gardner et al., Genome sequence of the human malaria parasite Plasmodium falciparum, Nature 419 (6906): 498-511 (2002). Plasmodium falciparum includes any strain thereof which exhibits sensitivity to an antigen-binding protein of the present invention, e.g., D10, Dd2, 7G8, W2-mef, 3D7, HB3, FCR-1/FVO, Cam3.II or RF7. “Plasmodium falciparum infection” refers to the invasion and multiplication of Plasmodium falciparum in the body of a subject. The present invention provides various antigen-binding proteins which are effective for treatment or prophylaxis of Plasmodium falciparum infection.

An anti-PfRH5 “antigen-binding protein” is a single polypeptide (e.g., an ScFv (single chain variable fragment)) or complex of more than one polypeptide (e.g., a tetrameric IgG antibody) that binds specifically to PfRH5 polypeptide, for example, an anti-PfRH5 antibody or antigen-binding fragment whether monospecific or multispecific (e.g., bispecific) or monovalent or multivalent (e.g., bivalent). A monovalent antigen-binding protein has a single antigen-binding domain whereas a bivalent antigen-binding protein has two antigen-binding domains.

Basigin (BSG, extracellular matrix metalloproteinase inducer, EMMPRIN, CD147) is a polypeptide which is a target on erythrocytes to which PfRH5 binds. In an embodiment of the invention, the amino acid sequence of basigin is set forth in Uniprot accession no. Q54A51. See e.g., Crosnier et al., Basigin is a receptor essential for erythrocyte invasion by Plasmodium falciparum, Nature. 2011 Nov. 9; 480 (7378): 534-7. In an embodiment of the invention, an antigen-binding protein of the present invention blocks binding between PfRH5 an BSG.

### Plasmodium Falciparum Reticulocyte-Binding Protein Homologue 5 (PfRH5)

Plasmodium falciparum Reticulocyte Binding Protein Homologue 5 (PfRH5) is a member of the super family of erythrocyte ligands referred to as the Reticulocyte Binding-Like proteins (RBLs). Evidence suggests that PfRH5 is essential for blood-stage growth of a Plasmodium falciparum infection. PfRH5 binds erythrocytes and is implicated in the species tropism of erythrocyte invasion. See e.g., Bustamante et al., Vaccine. 2013 Jan. 2; 31 (2): 373-379.

In an embodiment of the invention, PfRH5 comprises the amino acid sequence:

(SEQ ID NO: 361)
MIRIKKKLIL TIIYIHLFIL NRLSFENAIK KTKNQENNLT
LLPIKSTEEE KDDIKNGKDI KKEIDNDKEN IKTNNAKDHS
TYIKSYLNTN VNDGLKYLFI PSHNSFIKKY SVFNQINDGM
LLNEKNDVKN NEDYKNVDYK NVNFLQYHFK ELSNYNIANS
IDILQEKEGH LDFVIIPHYT FLDYYKHLSY NSIYHKSSTY
GKCIAVDAFI KKINETYDKV KSKCNDIKND LIATIKKLEH
PYDINNKNDD SYRYDISEEI DDKSEETDDE TEEVEDSIQD
TDSNHTPSNK KKNDLMNRTF KKMMDEYNTK KKKLIKCIKN
HENDFNKICM DMKNYGTNLF EQLSCYNNNF CNTNGIRYHY
DEYIHKLILS VKSKNLNKDL SDMTNILQQS ELLLTNLNKK
MGSYIYIDTI KFIHKEMKHI FNRIEYHTKI INDKTKIIQD
KIKLNIWRTF QKDELLKRIL DMSNEYSLFI TSDHLRQMLY
NTFYSKEKHL NNIFHHLIYV LQMKFNDVPI KMEYFQTYKK
NKPLTQ

The polypeptide “PfRH5ΔNL.6his” (PfRH5 (K140-Q526; del M1-Y139; del K247-L295; T216A; T299A)) which lacks the N-terminal residues 1-139 and residues 247-295 and has mutations T216A and T299A as well as a C-terminal His6 tag forms part of the present invention along with polynucleotides encoding the polypeptide. In an embodiment of the invention, the PfRH5ΔNL.6his polypeptide comprises the amino acid sequence set forth in SEQ ID NO: 362:

(SEQ ID NO: 362)
KNVNFLQYHFKELSNYNIANSIDILQEKEGHLDFVIIPHYTFLDYYKHLS
YNSIYHKSSTYGKYIAVDAFIKKINEAYDKVKSKCNDIKNDLIATIKKLE
HPYDINNMNRAFKKMMDEYNTKKKKLIKCIKNHENDFNKICMDMKNYGTN
LFEQLSCYNNNFCNTNGIRYHYDEYIHKLILSVKSKNLNKDLSDMTNILQ
QSELLLTNLNKKMGSYIYIDTIKFIHKEMKHIFNRIEYHTKIINDKTKII
QDKIKLNIWRTFQKDELLKRILDMSNEYSLFITSDHLRQMLYNTFYSKEK
HLNNIFHHLIYVLQMKFNDVPIKMEYFQTYKKNKPLTQHHHHHH.

In an embodiment of the invention, the polypeptide is in a crystallized form or a non-crystallized form.

### Antibodies and Antigen-Binding Fragments

The present invention provides antigen-binding proteins, such as antibodies and antigen-binding fragments thereof, that specifically bind to PfRH5 protein or an antigenic fragment thereof. Immunoglobulin chains of the present invention are described herein in Example 1 at Table 1-1.

The term “antibody”, as used herein, refers to immunoglobulin molecules comprising four polypeptide chains, two heavy chains (HCs) and two light chains (LCs) inter-connected by disulfide bonds (i.e., “full antibody molecules”), as well as multimers thereof (e.g. IgM)—for example, H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2, H1H29214P2; or H1H29215P2. Each heavy chain (HC) comprises a heavy chain variable region (“HCVR” or “V_{H}”) (e.g., SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354) and a heavy chain constant region (comprised of domains CH1, CH2 and CH3). Each light chain (LC) is comprised of a light chain variable region (“LCVR or “V_{L}”) (e.g., SEQ ID NO: 10, 26, 42, 58, 74, 90, 106, 122, 138, 154, 170, 186, 202, 218 or 306) and a light chain constant region (CL). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) are identical to the human germline sequences, or are naturally or artificially modified.

Typically, the variable domains of both the heavy and light immunoglobulin chains comprise three hypervariable regions, also called complementarity determining regions (CDRs), located within relatively conserved framework regions (FR). In general, from N-terminal to C-terminal, both light and heavy chains variable domains comprise FR1, CDR1, FR2, CDR2, FR3, CDR3 and FR4. In an embodiment of the invention, the assignment of amino acids to each domain is in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

The present invention includes monoclonal anti-PfRH5 antigen-binding proteins, e.g., antibodies and antigen-binding fragments thereof, as well as monoclonal compositions comprising a plurality of isolated monoclonal antigen-binding proteins. The term “monoclonal antibody”, as used herein, refers to a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment of the invention, an anti-PfRH5 antigen-binding protein, e.g., antibody or antigen-binding fragment comprises a heavy chain constant domain, e.g., of the type IgA (e.g., IgA1 or IgA2), IgD, IgE, IgG (e.g., IgG1, IgG2, IgG3 and IgG4) or IgM. In an embodiment of the invention, an antigen-binding protein, e.g., antibody or antigen-binding fragment, comprises a light chain constant domain, e.g., of the type kappa or lambda.

The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See e.g., U.S. Pat. Nos. 8,502,018, 6,596,541 or U.S. Pat. No. 5,789,215. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include antibodies isolated from or generated in a human subject.

The present invention includes anti-PfRH5 chimeric antigen-binding proteins, e.g., antibodies and antigen-binding fragments thereof, and methods of use thereof. As used herein, a “chimeric antibody” is an antibody having the variable domain from a first antibody and the constant domain from a second antibody, where the first and second antibodies are from different species. (see e.g., U.S. Pat. No. 4,816,567; and Morrison et al., (1984) Proc. Natl. Acad. Sci. USA 81:6851-6855).

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) expression system or isolated from a recombinant combinatorial human antibody library.

Recombinant anti-PfRH5 antigen-binding proteins, e.g., antibodies and antigen-binding fragments, disclosed herein may also be produced in an E. coli/T7 expression system. In this embodiment, nucleic acids encoding the anti-PfRH5 antibody immunoglobulin molecules of the invention (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2, H1H29214P2; or H1H29215P2) may be inserted into a pET-based plasmid and expressed in the E. coli/T7 system. For example, the present invention includes methods for expressing an antibody or antigen-binding fragment thereof or immunoglobulin chain thereof in a host cell (e.g., bacterial host cell such as E. coli such as BL21 or BL21DE3) comprising expressing T7 RNA polymerase in the cell which also includes a polynucleotide encoding an immunoglobulin chain that is operably linked to a T7 promoter. For example, in an embodiment of the invention, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside). See U.S. Pat. Nos. 4,952,496 and 5,693,489 or Studier & Moffatt, Use of bacteriophage T7 RNA polymerase to direct selective high-level expression of cloned genes, J. Mol. Biol. 1986 May 5; 189 (1): 113-30.

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

Polynucleotides encoding the immunoglobulins set forth herein (e.g., comprising an nucleotide sequence set forth in SEQ ID NO: 1, 3, 5, 7, 9, 11, 13, 17, 19, 21, 23, 25, 27, 29, 33, 35, 37, 39, 41, 43, 45, 49, 51, 53, 55, 57, 59, 61, 65, 67, 69, 71, 73, 75, 77, 81, 83, 85, 87, 89, 91, 93, 97, 99, 101, 103, 105, 107, 109, 113, 115, 117, 119, 121, 123, 125, 129, 131, 133, 135, 137, 139, 141, 145, 147, 149, 151, 153, 155, 157, 161, 163, 165, 167, 169, 171, 173, 177, 179, 181, 183, 185, 187, 189, 193, 195, 197, 199, 201, 203, 205, 209, 211, 213, 215, 217, 219, 221, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 313, 315, 317, 319, 321, 323, 325, 327, 329, 331, 333, 335, 337, 339, 341, 343, 345, 347, 349, 351, 353, 355, 357 and 359) which are in a vector and/or operably linked to an expression control sequence such as a promoter form part of the present invention. A promoter may be, for example, a CMV promoter (e.g., a human cytomegalovirus (CMV) major immediate-early (MIE) promoter or a mouse CMV promoter) or an SV40 promoter (e.g., SV40 early promoter). A vector may be a plasmid (e.g., a circular plasmid or a linearized plasmid) or a viral vector which may be maintained ectopically in a host cell or integrated into a host chromosome. Such host cells form part of the present invention.

A polynucleotide is “operably linked” when it is placed into a functional relationship with another nucleic acid sequence. For example, a promoter or enhancer is operably linked to a coding sequence if it affects the transcription of the sequence. Generally, but not always, “operably linked” means that the DNA sequences being linked are contiguous, and, in the case of a secretory leader, contiguous and in reading phase. However, enhancers do not have to be contiguous.

Thus, the present invention includes recombinant methods for making an anti-PfRH5 antigen-binding protein, such as an antibody or antigen-binding fragment thereof of the present invention, or an immunoglobulin chain thereof, comprising (i) introducing one or more polynucleotides (e.g., including the nucleotide sequence in any one or more of SEQ ID NOs: 1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289, 297, 305, 313, 321, 329, 337, 345 and/or 353) encoding light and/or heavy immunoglobulin chains of the antigen-binding protein, e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2, H1H29214P2; or H1H29215P2, for example, wherein the polynucleotide is in a vector; and/or integrated into a host cell chromosome and/or is operably linked to a promoter; (ii) culturing the host cell (e.g., CHO or Pichia or Pichia pastoris) under conditions favorable to expression of the polynucleotide and, (iii) optionally, isolating the antigen-binding protein, (e.g., antibody or fragment) or chain from the host cell and/or medium in which the host cell is grown. When making an antigen-binding protein (e.g., antibody or antigen-binding fragment) comprising more than one immunoglobulin chain, e.g., an antibody that comprises two heavy immunoglobulin chains and two light immunoglobulin chains, co-expression of the chains in a single host cell leads to association of the chains, e.g., in the cell or on the cell surface or outside the cell if such chains are secreted, so as to form the antigen-binding protein (e.g., antibody or antigen-binding fragment). The methods of the present invention include those wherein only a heavy immunoglobulin chain or only a light immunoglobulin chain or both (e.g., any of those discussed herein including mature fragments and/or variable domains thereof) are expressed in a cell. Such single chains are useful, for example, as intermediates in the expression of an antibody or antigen-binding fragment that includes such a chain. For example, the present invention also includes anti-PfRH5 antigen-binding proteins, such as antibodies and antigen-binding fragments thereof, comprising a heavy chain immunoglobulin (or variable domain thereof or comprising the CDRs thereof) encoded by a polynucleotide comprising the nucleotide sequences set forth in SEQ ID NO: 1 17, 33, 49, 65, 81, 97, 113, 129, 145, 161, 177, 193, 209, 225, 233, 241, 249, 257, 265, 273, 281, 289, 297, 313, 321, 329, 337, 345 or 353 and a light chain immunoglobulin (or variable domain thereof or comprising the CDRs thereof) encoded by the nucleotide sequence set forth in SEQ ID NO: 9, 25, 41, 57, 73, 89, 105, 121, 137, 153, 169, 185, 201, 217 or 305, which are the product of such production methods, and, optionally, the purification methods set forth herein. For example, in an embodiment of the invention, the product of the method is an anti-PfRH5 antigen-binding protein which is an antibody or fragment comprising a V_{H} comprising the amino acid sequence set forth in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354 and a V_{L} comprising the amino acid sequence set forth in SEQ ID NO: 10, 26, 42, 58, 74, 90, 106, 122, 138, 154, 170, 186, 202, 218 or 306.

In an embodiment of the invention, a method for making an anti-PfRH5 antigen-binding protein, e.g., antibody or antigen-binding fragment thereof, includes a method of purifying the antigen-binding protein, e.g., by column chromatography, precipitation, and/or filtration. The product of such a method also forms part of the present invention.

Eukaryotic and prokaryotic host cells, including mammalian cells, may be used as hosts for expression of an anti-PfRH5 antigen-binding protein (e.g., antibody or antigen-binding fragment thereof). Such host cells are well known in the art and many are available from the American Type Culture Collection (ATCC). These host cells include, inter alia, Chinese hamster ovary (CHO) cells, NSO, SP2 cells, Hela cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present invention includes an isolated host cell (e.g., a CHO cell) comprising an antigen-binding protein, such as H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2, H1H29214P2; or H1H29215P2; or one or more polynucleotides encoding an immunoglobulin chain or chains thereof.

The present invention also includes a Plasmodium falciparum cell which is expressing PfRH5 which is bound by an antigen-binding protein of the present invention e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2, H1H29214P2; or H1H29215P2, e.g., wherein the cell is in the body of a subject or is in vitro.

The term “specifically binds” refers to those antigen-binding proteins (e.g., mAbs) having a binding affinity to an antigen, such as PfRH5 protein (e.g., PfRH5ΔNL.6his), expressed as K_{D}, of at least about 108 M (e.g., any K_{D} set forth in Table 5-1 or 5-2 herein), as measured by real-time, label free bio-layer interferometry assay, for example, at 25° C. or 37° C., e.g., an Octet® HTX biosensor, or by surface plasmon resonance, e.g., BIACORE™, or by ELISA (enzyme linked immunosorbent assay). The present invention includes antigen-binding proteins that specifically bind to PfRH5 protein. In an embodiment of the invention, an antigen-binding protein comprises a Kₐ, K_{d} and/or t_{1/2} as set forth in Table 5-1 or 5-2 herein.

The terms “antigen-binding portion” or “antigen-binding fragment” of an antibody or antigen-binding protein, and the like, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments and (vii) constrained FR3-CDR3-FR4 peptides (e.g., comprising a FR3, FR4 and CDR-H3 or CDR-L3 as set forth herein). Other engineered molecules, such as domain-specific antibodies, single domain antibodies, domain-deleted antibodies, chimeric antibodies, CDR-grafted antibodies, diabodies, triabodies, tetrabodies, minibodies and small modular immunopharmaceuticals (SMIPs) are also encompassed within the expression “antigen-binding fragment,” as used herein. In an embodiment of the invention, the antigen-binding fragment comprises three or more CDRs of H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2, H1H29214P2; or H1H29215P2 (e.g., CDR-H1, CDR-H2 and CDR-H3; and/or CDR-L1, CDR-L2 and CDR-L3).

An antigen-binding fragment of an antibody will, in an embodiment of the invention, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

Antigen-binding proteins (e.g., antibodies and antigen-binding fragments) may be monospecific or multi-specific (e.g., bispecific). Multispecific antigen-binding proteins are discussed further herein.

In an embodiment of the invention, antigen-binding proteins of the present invention (e.g., an antibody or antibody fragment) may be conjugated to a moiety such as a therapeutic moiety (“immunoconjugate”), such as an anti-malarial drug, a second anti-PfRH5 antibody, or any other therapeutic moiety useful for treating a Plasmodium falciparum infection. See e.g., below.

The present invention also provides a complex comprising an anti-PfRH5 antigen-binding protein, e.g., antibody or antigen-binding fragment, discussed herein complexed with PfRH5 polypeptide or an antigenic fragment thereof and/or with a secondary antibody or antigen-binding fragment thereof (e.g., detectably labeled secondary antibody) that binds specifically to the anti-PfRH5 antibody or fragment. In an embodiment of the invention, the complex is in vitro (e.g., is immobilized to a solid substrate) or is in the body of a subject. In an embodiment of the invention, the PfRH5 is immobilized to a solid substrate (e.g., a lateral flow test strip) or is on the surface of a cell such as Plasmodium falciparum. Immobilized anti-PfRH5 antibodies and antigen-binding fragments thereof which are covalently linked to an insoluble matrix material (e.g., glass or polysaccharide such as agarose or sepharose, e.g., a bead or other particle thereof) are also part of the present invention; optionally, wherein the immobilized antibody is complexed with PfRH5 or antigenic fragment thereof or a secondary antibody or fragment thereof.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antigen-binding proteins (e.g., antibodies or antigen-binding fragments).

The present invention includes antigen-binding proteins, e.g., antibodies or antigen-binding fragments, that bind to the same epitope as an antigen-binding protein of the present invention (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2, H1H29214P2; or H1H29215P2). For example, the present invention includes antigen-binding proteins that bind to a PfRH5 variant epitope (e.g., PfRH5ΔNL.6his) lacking amino acids M1-Y139 and including residues K140-Q526 but lacking K247-L295 and having the mutations T216A and T299A (optionally, including a HisX6 tag).

The term “epitope” refers to an antigenic determinant (e.g., on PfRH5 polypeptide) that interacts with a specific antigen-binding site of an antigen-binding protein, e.g., a variable region of an antibody molecule, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Methods for determining the epitope of an antigen-binding protein, e.g., antibody or fragment or polypeptide, include alanine scanning mutational analysis, peptide blot analysis (Reineke (2004) Methods Mol. Biol. 248:443-63), peptide cleavage analysis, crystallographic studies and NMR analysis. In addition, methods such as epitope excision, epitope extraction and chemical modification of antigens can be employed (Tomer (2000) Prot. Sci. 9:487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267:252-259; Engen and Smith (2001) Anal. Chem. 73: 256A-265A.

The present invention includes antigen-binding proteins that compete for binding to PfRH5, e.g., a variant PfRH5 epitope as discussed herein, with an antigen-binding protein of the present invention, e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2, H1H29214P2; or H1H29215P2. The term “competes” as used herein, refers to an antigen-binding protein (e.g., antibody or antigen-binding fragment thereof) that binds to an antigen (e.g., PfRH5 or PfRH5ΔNL.6his) and inhibits or blocks the binding of another antigen-binding protein (e.g., antibody or antigen-binding fragment thereof) to the antigen. The term also includes competition between two antigen-binding proteins e.g., antibodies, in both orientations, i.e., a first antibody that binds and blocks binding of second antibody and vice versa. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. In an embodiment of the invention, competition between a first and second anti-PfRH5 antigen-binding protein (e.g., antibody) is determined by measuring the ability of an immobilized first anti-PfRH5 antigen-binding protein (e.g., antibody) (not initially complexed with PfRH5 protein) to bind to PfRH5 protein or a fragment thereof complexed with a second anti-PfRH5 antigen-binding protein (e.g., antibody). A reduction in the ability of the first anti-PfRH5 antigen-binding protein (e.g., antibody) to bind to the complexed PfRH5 protein, relative to uncomplexed PfRH5 protein, indicates that the first and second anti-PfRH5 antigen-binding proteins (e.g., antibodies) compete. The degree of competition can be expressed as a percentage of the reduction in binding. Such competition can be measured using a real time, label-free bio-layer interferometry assay, e.g., on an Octet RED384 biosensor (Pall ForteBio Corp.), ELISA (enzyme-linked immunosorbent assays) or SPR (surface plasmon resonance).

Binding competition between anti-PfRH5 antigen-binding proteins (e.g., monoclonal antibodies (mAbs)) can be determined using a real time, label-free bio-layer interferometry assay on an Octet RED384 biosensor (Pall ForteBio Corp.). For example, to determine competition between two anti-PfRH5 monoclonal antibodies, the anti-PfRH5 mAb can be first captured onto anti-hFc antibody coated Octet biosensor tips (Pall ForteBio Corp., #18-5060) by submerging the tips into a solution of anti-PfRH5 mAb (subsequently referred to as “mAb1”). As a positive-control for blocking, the antibody captured biosensor tips can then be saturated with a known blocking isotype control mAb (subsequently referred to as “blocking mAb”) by dipping into a solution of blocking mAb. To determine if mAb2 competes with mAb1, the biosensor tips can then be subsequently dipped into a complexed solution of PfRH5 polypeptide and a second anti-PfRH5 mAb (subsequently referred to as “mAb2”), that had been pre-incubated for a period of time and binding of mAb1 to the PfRH5 polypeptide can be determined. The biosensor tips can be washed in buffer in between every step of the experiment. The real-time binding response can be monitored during the course of the experiment and the binding response at the end of every step can be recorded.

In an embodiment of the invention, the competition assay is conducted on an biosensor platform (e.g., Octet HTX), wherein one antibody is bound/complexed to PfRH5 polypeptide which has been bound to a sensor tip and binding of a second antibody to the PfRH5 is then assessed. The ability of the second antibody to bind to the pre-complexed PfRH5 polypeptide can be assessed and, if reduced binding (e.g., relative to PfRH5 not complexed with a first antibody) or an absence of binding of the second antibody is detected, then the first and second antibodies are determined to compete for PfRH5 polypeptide binding. In an embodiment of the invention, the assay is conducted at 25° C. and pH about 7, e.g., 7.4, e.g., in the presence of buffer (e.g., HEPES), salt (e.g., NaCl), EDTA, surfactant (e.g., Tween-20) and/or a non-specific protein (e.g., bovine serum albumin). In an embodiment of the invention, binding to a PfRH5 variant (e.g., PfRH5ΔNL.6his) is assessed in the competition assay, e.g., wherein the variant is PfRH5 lacking amino acids M1-Y139 and including residues K140-Q526 but lacking K247-L295 and having the mutations T216A and T299A (optionally, including a HisX6 tag). A HisX6 or His6 tag is a tag that includes HHHHHH (SEQ ID NO: 365).

Typically, an antibody or antigen-binding fragment of the invention which is modified in some way retains the ability to specifically bind to PfRH5, e.g., retains at least 10% of its PfRH5 binding activity (when compared to the parental antibody) when that activity is expressed on a molar basis. Preferably, an antibody or antigen-binding fragment of the invention retains at least 20%, 50%, 70%, 80%, 90%, 95% or 100% or more of the PfRH5 binding affinity as the parental antibody. It is also intended that an antibody or antigen-binding fragment of the invention may include conservative or non-conservative amino acid substitutions (referred to as “conservative variants” or “function conserved variants” of the antibody) that do not substantially alter its biologic activity.

Anti-PfRH5 antigen-binding proteins of the present invention may comprise variants of the immunoglobulin chains whose amino acid and nucleotide sequences are specifically set forth herein.

A “variant” of a polypeptide, such as an immunoglobulin chain (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2 V_{H}, V_{L}, HC or LC), refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346, 354, 10, 26, 42, 58, 74, 90, 106, 122, 138, 154, 170, 186, 202, 218 or 306); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein (e.g., SEQ ID NO: 1, 17, 33, 49, 65, 81, 97, 113, 129, 145, 161, 177, 193, 209, 225, 233, 241, 249, 257, 265, 273, 281, 289, 297, 313, 321, 329, 337, 345, 353, 9, 25, 41, 57, 73, 89, 105, 121, 137, 153, 169, 185, 201, 217 or 305); when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1,−2; gap costs: linear).

Anti-PfRH5 antigen-binding proteins, e.g., antibodies and antigen-binding fragments thereof of the present invention, in an embodiment of the invention, include a heavy chain immunoglobulin variable region having at least 70% (e.g., 80%, 85%, 90%, 95%, 99%) amino acid sequence identity to that of an immunoglobulin heavy chain whose amino acid sequence is set forth herein, e.g., in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354; and/or a light chain immunoglobulin variable region having at least 70% (e.g., 80%, 85%, 90%, 95%, 99%) amino acid sequence identity to an immunoglobulin light chain whose amino acid sequence is set forth herein, e.g., in SEQ ID NO: 10, 26, 42, 58, 74, 90, 106, 122, 138, 154, 170, 186, 202, 218 or 306.

In addition, an anti-PfRH5 antigen-binding protein of the present invention may include a variant immunoglobulin polypeptide comprising an amino acid sequence that is set forth herein except for one or more (e.g., 1, 2, 3, 4, 5, 6, 7, 8, 9 or 10) mutations such as, for example, missense mutations (e.g., conservative substitutions), non-sense mutations, deletions, or insertions. For example, the present invention includes antigen-binding proteins which include an immunoglobulin light chain variant comprising the amino acid sequence set forth in SEQ ID NO: 10, 26, 42, 58, 74, 90, 106, 122, 138, 154, 170, 186, 202, 218 or 306 but having one or more of such mutations and/or an immunoglobulin heavy chain variant comprising the amino acid sequence set forth in SEQ ID NO: 2, 18, 34, 50, 66, 82, 98, 114, 130, 146, 162, 178, 194, 210, 226, 234, 242, 250, 258, 266, 274, 282, 290, 298, 314, 322, 330, 338, 346 or 354 but having one or more of such mutations. In an embodiment of the invention, an anti-PfRH5 antigen-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) relative to a sequence which is specifically set forth herein and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions) relative to a sequence which is specifically set forth herein.

The invention further provides variant anti-PfRH5 antigen-binding proteins, e.g., antibodies or antigen-binding fragments thereof, comprising one or more variant CDRs (e.g., any one or more of CDR-L1, CDR-L2, CDR-L3, CDR-H1, CDR-H2 and/or CDR-H3) that are set forth herein with at least 70%, 75%, 80%, 85%, 90%, 95%, 98%, 99% or 99.9% sequence identity or similarity thereto.

Embodiments of the present invention also include anti-PfRH5 antibodies and antigen-binding fragments thereof, that comprise variant immunoglobulin VHS and VLs; or HCs and LCs, which comprise an amino acid sequence having 70% or more (e.g., 80%, 85%, 90%, 95%, 97% or 99%) overall amino acid sequence identity or similarity to the amino acid sequences of the corresponding VHS, VLS, HCs or LCs specifically set forth herein, but wherein the CDR-L1, CDR-L2, CDR-L3, CDR-H1, CDR-H2 and CDR-H3 of such immunoglobulins are not variants and comprise the amino acid sequences set forth herein. Thus, in such embodiments, the CDRs within such antigen-binding proteins are not, themselves, variants.

A “conservatively modified variant” or a “conservative substitution” refers to a variant wherein there are one or more substitutions of amino acids in a polypeptide with other amino acids having similar characteristics (e.g. charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to significantly disrupt biological activity. Anti-PfRH5 antigen-binding proteins of the present invention may include immunoglobulin chains having an amino acid sequence set forth herein but having one or more conservatively modified variations.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45.

In an embodiment of the invention, an anti-PfRH5 antigen-binding protein of the present invention, e.g., comprising an immunoglobulin chain comprising a variant of an amino acid sequence set forth herein, exhibits one or more of the following functional properties:
Inhibits in vitro or in vivo growth of Plasmodium falciparum in human red blood cells, e.g., as measured by parasite lactate dehydrogenase (LDH) activity (e.g., at a rate of about 51 to about 69%, or up to 100% (e.g., when anti-PfRH5 antigen-binding protein is incubated with the cells for 96 hours) relative to uninfected red blood cells), for example, wherein the antigen-binding protein is H1H29100P, H1H29104P, H1H29127P, H1H29143P or any combination thereof of two of such proteins (see e.g., Table 2-1).
Inhibits growth of in vitro or in vivo Plasmodium falciparum strain 3D7 or 7G8 in human red blood cells, e.g., as measured by Parasite lactate dehydrogenase (LDH) activity, e.g., in the presence of chloroquine phosphate (CQ) (e.g., at a concentration of about 4.81 nM or 6.58 nM), for example, at a rate of about 34 to 61% in the absence of CQ, about 32 to 51% in the presence of 4.81 nM CQ or about 20% to 75% in the presence of 6.58 nM CQ.
Binds to PfRH5 polypeptide or an antigenic fragment thereof, e.g., PfRH5ΔNL.6his, with a K_{D} of about 4.72 pM to about 1.67 nM at 25° C. and/or of about 1.10 pM to about 1.10 nM at 37° C., e.g., as set forth in Tables 5-1 and 5-2 herein.
Inhibits growth of in vitro or in vivo Plasmodium falciparum strain D10, Dd2, 7G8, W2-mef, 3D7, HB3, FCR-1/FVO, Cam3.II or RF7 in human red blood cells, e.g., as measured by Parasite lactate dehydrogenase (LDH) activity, for example, at a concentration of about 666.67 nM at a rate as set forth in Table 4-2 relative to uninfected red blood cells.
Blocks binding of PfRH5 polypeptide (e.g., PfRH5ΔNL.6his, for example, at a concentration of about 0.5 nM or 2.0 nM) to basigin polypeptide (e.g., by about 1%, 10%, 20%, 30%, 40%, 50%, 60%, 70%, 80%, 90%, 95%, 99% or 100%), for example, binding of PfRH5 to basigin that is bound to a solid matrix (e.g., an ELISA plate) wherein the anti-PfRH5 antigen-binding protein is present at about 100 nM. For example, wherein basigin was amino acids Thr25-His205 thereof, e.g., expressed with a C-terminal linker, DIEGRMD (SEQ ID NO: 363), followed by a portion of the human IgG1 (Pro100-Lys330) and a 6X histidine tag.
Binds to the same epitope as H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2, for example, binds to PfRH5 lacking the amino-terminal residues M1-Y139 and including residues K140-Q526 but lacking K247-L295 and having the mutations T216A and T299A (PfRH5ΔNL.6his), e.g., further including C-terminal hexahistidine tag, for example, as measured by surface plasmon resonance.
Competes with H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2, H1H29214P2; or H1H29215P2 for binding to PfRH5 polypeptide, e.g., PfRH5ΔNL.6his.

The present invention includes a non-human primate (NHP) (e.g., monkey such as an Aotus monkey) whose body includes an anti-PfRH5 antigen-binding protein (e.g., antibody or antigen-binding fragment) such as H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2. For example, the non-human primate may have been injected with the antigen-binding protein or may be engineered to express the protein. In an embodiment of the invention, the non-human primate is Aotus nancymaae. In an embodiment of the invention, the non-human primate (e.g., monkey) is infected with Plasmodium falciparum (e.g., strain 3D7).

The present invention includes “neutralizing” or “antagonist” anti-PfRH5 antigen-binding proteins, e.g., antibody or antigen-binding fragment, which include molecules that inhibits an activity of PfRH5 to any detectable degree. For example, a neutralizing anti-PfRH5 antigen-binding protein may inhibit Plasmodium falciparum growth and/or block PfRH5/BSG binding.

“H1H29089P”; “H1H29094P”; “H1H29100P”; “H1H29104P”; “H1H29106P”; “H1H29109P”; “H1H29125P”; “H1H29127P”; “H1H29131P”; “H1H29134P”; “H1H29138P”; “H1H29141P”; “H1H29143P”; “H1H29146P2”; “H1H29147P2”; H1H29149P2″; “H1H29151P2”; “H1H29163P2”; “H1H29166P2”; “H1H29171P2”; “H1H29179P2”; “H1H29183P2”; “H1H29187P2”; “H1H29192P2”; “H1H29196P2”; “H1H29198P2”; “H1H29207P2”; “H1H29209P2”; “H1H29214P2”; or “H1H29215P2” refer to antigen-binding proteins, e.g., antibodies and antigen-binding fragments thereof (including multispecific antigen-binding proteins), comprising an immunoglobulin heavy chain variable region (V_{H}; or a variant thereof) and an immunoglobulin light chain variable region (V_{L}; or a variant thereof) which are set forth herein in Table 1-1; or that comprise a V_{H} that comprises the CDRs thereof (CDR-H1 (or a variant thereof), CDR-H2 (or a variant thereof) and CDR-H3 (or a variant thereof)) and/or a V_{L} that comprises the CDRs thereof (CDR-L1 (or a variant thereof), CDR-L2 (or a variant thereof) and CDR-L3 (or a variant thereof)), e.g., wherein the immunoglobulin chains, variable regions and/or CDRs comprise the specific amino acid sequences described below. In an embodiment of the invention, the V_{H} is linked to a constant heavy immunoglobulin chain (e.g., an IgG such as IgG1 or IgG4) and/or the V_{L} is linked to a constant light immunoglobulin chain (e.g., kappa or lambda).

Antibodies and antigen-binding fragments of the present invention comprise immunoglobulin chains including the amino acid sequences set forth herein as well as cellular and in vitro post-translational modifications to the antibody or fragment. For example, the present invention includes antibodies and antigen-binding fragments thereof that specifically bind to PfRH5 comprising heavy and/or light chain amino acid sequences set forth herein (e.g., CDR-H1, CDR-H2, CDR-H3, CDR-L1, CDR-L2 and/or CDR-L3) as well as antibodies and fragments wherein one or more amino acid residues is glycosylated, one or more Asn residues is deamidated, one or more residues (e.g., Met, Trp and/or His) is oxidized, the N-terminal Gln is pyroglutamate (pyroE) and/or the C-terminal Lysine is missing.

The present invention provides a vessel (e.g., a plastic or glass vial, e.g., with a cap or a chromatography column, hollow bore needle or a syringe cylinder) comprising an anti-PfRH5 antigen-binding protein of the present invention, e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2.

The present invention also provides an injection device comprising one or more antigen-binding proteins (e.g., antibody or antigen-binding fragment) that bind specifically to PfRH5, e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2, or a pharmaceutical composition thereof. The injection device may be packaged into a kit. An injection device is a device that introduces a substance into the body of a subject via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising the antibody or fragment or a pharmaceutical composition thereof), a needle for piecing skin and/or blood vessels for injection of the fluid; and a plunger for pushing the fluid out of the cylinder and through the needle bore.

The present invention further provides methods for administering an anti-PfRH5 antigen-binding protein of the present invention, e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2, to a subject, comprising introducing the antigen-binding protein into the body of the subject (e.g., a human), for example, parenterally. For example, the method comprises piercing the body of the subject with a needle of a syringe and injecting the antigen-binding protein into the body of the subject, e.g., into the vein, artery, tumor, muscular tissue or subcutis of the subject.

### Preparation of Human Antibodies

Methods for generating human antibodies in transgenic mice are known in the art. Any such known methods can be used in the context of the present invention to make human antibodies that specifically bind to PfRH5. An immunogen comprising any one of the following can be used to generate antibodies that specifically bind to PfRH5. In certain embodiments of the invention, the antibodies of the invention are obtained from mice immunized with a full length, native PfRH5, or with a live attenuated or inactivated virus, or with DNA encoding the protein or fragment thereof. Alternatively, the PfRH5 protein or a fragment thereof may be produced using standard biochemical techniques and modified and used as immunogen. In one embodiment of the invention, the immunogen is a recombinantly produced PfRH5 protein or fragment thereof. In certain embodiments of the invention, the immunogen may be a PfRH5 polypeptide vaccine. In certain embodiments, one or more booster injections may be administered. In certain embodiments, the immunogen may be a recombinant PfRH5 polypeptide expressed in E. coli or in any other eukaryotic or mammalian cells such as Chinese hamster ovary (CHO) cells.

Using VELOCIMMUNE® technology (see, for example, U.S. Pat. No. 6,596,541, Regeneron Pharmaceuticals, VELOCIMMUNE®), high affinity chimeric antibodies to PfRH5 can be initially isolated having human variable regions and mouse constant regions. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Antibodies of interest may also be isolated from mouse B-cells. Briefly, splenocytes are harvested from each mouse and B-cells are sorted (as described in US 2007/0280945A1, for example) by FACS using the antigen of interest as the sorting reagent that binds and identifies reactive antibodies (antigen-positive B cells). Various methods of identifying and sorting antigen-positive B cells, as well as constructing immunoglobulin gene expression cassettes by PCR for preparation of cells expressing recombinant antibodies, are well-known in the art. See e.g. WO20141460741, U.S. Pat. No. 7,884,054B2, and Liao, et al. June 2009. High-Throughput Isolation of Immunoglobulin Genes from Single Human B Cells and Expression as Monoclonal Antibodies. J Virol Methods 158 (1-2): 171-9.

Initially, high affinity chimeric antibodies are isolated having a human variable region and a mouse constant region. The antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

### Anti-PfRH5 Antibodies Comprising Fc Variants

According to certain embodiments of the present invention, anti-PfRH5 antigen-binding proteins, e.g., antibodies or antigen-binding fragments, are provided comprising an Fc domain comprising one or more mutations, which, for example, enhance or diminish antibody binding to the FcRn receptor, e.g., at acidic pH as compared to neutral pH. For example, the present invention includes anti-PfRH5 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-PfRH5 antigen-binding proteins, e.g., antibodies or antigen-binding fragments, comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 2571 and 3111 (e.g., P2571 and Q3111); 2571 and 434H (e.g., P2571 and N434H); 376V and 434H (e.g., D376V and N434H); 307A, 380A and 434A (e.g., T307A, E380A and N434A); and 433K and 434F (e.g., H433K and N434F).

Anti-PfRH5 antigen-binding proteins, e.g., antibodies and antigen-binding fragments thereof, that comprise a V_{H} and/or V_{L} as set forth herein comprising any possible combinations of the foregoing Fc domain mutations, are contemplated within the scope of the present invention.

The present invention also includes anti-PfRH5 antigen-binding proteins, antibodies or antigen-binding fragments, comprising a V_{H} set forth herein and a chimeric heavy chain constant (C_{H}) region, wherein the chimeric C_{H} region comprises segments derived from the C_{H} regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., WO2014/022540).

### Multispecific Antigen-Binding Proteins

The present invention includes anti-PfRH5 antigen-binding proteins, e.g., antibodies and antigen-binding fragments thereof, as well as methods of use thereof and methods of making such antigen-binding proteins. The term “anti-PfRH5” antigen-binding proteins, e.g., antibodies or antigen-binding fragments, includes multispecific (e.g., bispecific or biparatopic) molecules that include at least one first antigen-binding domain that specifically binds to PfRH5 (e.g., an antigen-binding domain from H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2) and at least one second antigen-binding domain that binds to a different antigen or to an epitope in PfRH5 which is different from that of the first antigen-binding domain (e.g., CD3, CD16, BSG (basigin), EXP1, MSP1, MSP2, MSPMSP3, MSP4, MSP5, MSP6, MSP7, MSP9, MSP10 GLURP, Sera, RAMA, SEA, AMA1, MTRAP, PTRAMP, ASP, RH1, RH2a, RH2b, RH4, RAP1, RAP2, RAP3, RhopH1, RhopH2, RhopH3, EMA175, EMA140 and/or EBA181). In an embodiment of the invention, the first and second epitopes overlap. In another embodiment of the invention, the first and second epitopes do not overlap.

In an embodiment of the invention, a multispecific antigen-binding protein binds to PfRH5 and to an antigen which causes activation of the immune cells such as cytotoxic T cells, NK cells, mononuclear phagocytes or neutrophils, e.g., CD3 or CD16.

“H1H29089P”; “H1H29094P”; “H1H29100P”; “H1H29104P”; “H1H29106P”; “H1H29109P”; “H1H29125P”; “H1H29127P”; “H1H29131P”; “H1H29134P”; “H1H29138P”; “H1H29141P”; “H1H29143P”; “H1H29146P2”; “H1H29147P2”; “H1H29149P2”; “H1H29151P2”; “H1H29163P2”; “H1H29166P2”; “H1H29171P2”; “H1H29179P2”; “H1H29183P2”; “H1H29187P2”; “H1H29192P2”; “H1H29196P2”; “H1H29198P2”; “H1H29207P2”; “H1H29214P2”; or “H1H29215P2” includes a multispecific molecules, e.g., antibodies or antigen-binding fragments, that include the HCDRs and LCDRs, V_{H} and V_{L}, or HC and LC of H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2, respectively (including variants thereof as set forth herein) and one or more antigen-binding domains that bind to a different epitope.

In an embodiment of the invention, an antigen-binding domain that binds specifically to PfRH5, which may be included in a multispecific molecule, comprises:

(1)
(i) a heavy chain variable domain sequence that comprises CDR-H1, CDR-H2 and CDR-H3 from an immunoglobulin heavy chain selected from: H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; and H1H29215P2, and
(ii) a light chain variable domain sequence that comprises CDR-L1, CDR-L2 and CDR-L3 from an immunoglobulin heavy chain selected from: H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; and H1H29215P2, respectively;
   or,
   (2)
(i) a heavy chain variable domain (V_{H}) sequence selected from: H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; and H1H29215P2; and
(ii) a light chain variable domain (V_{L}) sequence selected from: H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; and H1H29215P2, respectively;
and one or more antigen-binding domains that bind to a different epitope.

In an embodiment of the invention, the multispecific antibody or fragment includes more than two different binding specificities (e.g., a trispecific molecule), for example, one or more additional antigen-binding domains which are the same or different from the first and/or second antigen-binding domain.

In one embodiment of the invention, a bispecific antigen-binding fragment comprises a first scFv (e.g., comprising V_{H} and V_{L} of H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; and H1H29215P2) having binding specificity for a first epitope (e.g., PfRH5) and a second scFv having binding specificity for a second, different epitope. For example, in an embodiment of the invention, the first and second scFv are tethered with a linker, e.g., a peptide linker (e.g., a GS linker such as (GGGGS) n (SEQ ID NO: 364) wherein n is, for example, 1, 2, 3, 4, 5, 6, 7, 8, 9 or 10). Other bispecific antigen-binding fragments include an F(ab) 2 of a bispecific IgG antibody which comprises the heavy and light chain CDRs of H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2 H1H29209P2; H1H29214P2; and H1H29215P2 and of another antibody that binds to a different epitope.

### Immunoconjugates

The invention encompasses anti-PfRH5 antigen-binding proteins, e.g., antibodies or antigen-binding fragments, conjugated to another moiety, e.g., a therapeutic moiety (an “immunoconjugate”), such as a toxoid (e.g., diptheria toxoid or tetanus toxoid) or an anti-parasitic drug to treat Plasmodium falciparum infection. In an embodiment of the invention, an anti-PfRH5 antibody or fragment is conjugated to any of the further therapeutic agents set forth herein. As used herein, the term “immunoconjugate” refers to an antigen-binding protein, e.g., an antibody or antigen-binding fragment, which is chemically or biologically linked to another molecule.

### Therapeutic Methods

The present invention provides methods for treating or preventing Plasmodium falciparum infection (e.g., malaria) by administering a therapeutically effective amount of anti-PfRH5 antigen-binding protein, e.g., antibody or antigen-binding fragment, (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2) to a subject (e.g., a human) in need of such treatment or prevention. “Malaria” is a disease, frequently transmitted by the bite of an infected female mosquito (e.g., Anopheles mosquitos), caused by infection of a host with the parasite Plasmodium falciparum. The term “Plasmodium falciparum infection” refers to invasion of the body of a subject with Plasmodium falciparum and encompasses malaria.

An effective or therapeutically effective dose of anti-PfRH5 antigen-binding protein, e.g., antibody or antigen-binding fragment (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2), for treating or preventing a Plasmodium falciparum infection refers to the amount of the antibody or fragment sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment of the invention, an effective or therapeutically effective dose of anti-PfRH5 antigen-binding protein, e.g., antibody or antigen-binding fragment thereof, of the present invention, for treating or preventing Plasmodium falciparum infection, e.g., in an adult human subject, is about 1 mg/kg to 150 mg/kg. Depending on the severity of the infection, the frequency and the duration of the treatment can be adjusted. In certain embodiments, the antigen-binding protein of the present invention can be administered at an initial dose, followed by one or more secondary doses. In certain embodiments, the initial dose may be followed by administration of a second or a plurality of subsequent doses of antigen-binding protein in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

As used herein, the term “subject” refers to a mammal (e.g., rat, mouse, cat, dog, cow, sheep, horse, goat, rabbit), preferably a human, for example, in need of prevention and/or treatment of a Plasmodium falciparum infection. A subject may have a Plasmodium falciparum infection or be predisposed to developing a Plasmodium falciparum infection or be at elevated risk of developing such an infection. Subjects predisposed to developing a Plasmodium falciparum infection or subjects who may be at elevated risk for contracting a Plasmodium falciparum infection, include those subjects with compromised immune systems, e.g., because of autoimmune disease, those persons receiving immunosuppressive therapy, those persons afflicted with human immunodeficiency syndrome (HIV) or acquired immune deficiency syndrome (AIDS), certain forms of anemia that deplete or destroy white blood cells, those persons receiving radiation or chemotherapy, or those persons afflicted with an inflammatory disorder. Additionally, subjects of extreme young or old age may be predisposed. Any person who comes into contact with or close proximity to mosquitos, especially in the tropics, South America, Central America, Africa, South East Asia, and the Eastern Mediterranean Region, has an increased risk of developing Plasmodium falciparum infection.

“Treat” or “treating” means to administer an anti-PfRH5 antigen-binding protein, e.g., antibody or antigen-binding fragment of the present invention (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2), to a subject having Plasmodium falciparum infection, such that one or more signs or symptoms of the infection in the subject are reduced or eliminated, e.g., wherein Plasmodium falciparum is reduced or substantially eliminated (e.g., completely eliminated) from the body of the subject.

Signs and symptoms of Plasmodium falciparum infection include:
Anemia;
Bloody stools;
Chills, fever, sweating;
Coma;
Convulsions;
Headache;
Jaundice;
Muscle pain;
Nausea and vomiting;
Enlarged spleen;
Jaundice;
Enlargement of the liver;
Increased respiratory rate;
Plasmodium falciparum in the blood stream, liver or erythrocytes;
Anemia;
Hemolysis;
Free hemoglobin in the blood stream;
Hemoglobinuria;
Acute kidney failure;
Acute respiratory distress syndrome (ARDS);
Low blood pressure;
Metabolic acidosis; and
Hypoglycemia.

The present invention also encompasses prophylactically administering an anti-PfRH5 antigen-binding protein, e.g., antibody or antigen-binding fragment thereof of the present invention (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2), to a subject who is at risk (e.g., predisposed or at an elevated risk) of Plasmodium falciparum infection so as to prevent such infection. “Prevent” or “preventing” means to administer an anti-PfRH5 antigen-binding protein, e.g., antibody or antigen-binding fragment of the present invention, to a subject who is not infected with Plasmodium falciparum such that manifestation of the infection in the body of a subject is inhibited or decreased in likelihood or decreased in severity if infection does occur.

### Combinations and Pharmaceutical Compositions

The present invention provides compositions that include anti-PfRH5 antigen-binding proteins and one or more ingredients; as well as methods of use thereof and methods of making such compositions.

To prepare pharmaceutical compositions of the anti-PfRH5 antigen-binding proteins, e.g., antibodies and antigen-binding fragments thereof (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2), antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In an embodiment of the invention, the pharmaceutical composition is sterile. Such compositions are part of the present invention.

Pharmaceutical compositions of the present invention include pharmaceutically acceptable carriers, diluents, excipients and/or stabilizers, such as, for example, water, buffering agents, stabilizing agents, preservatives, isotonifiers, non-ionic detergents, antioxidants and/or other miscellaneous additives.

The scope of the present invention includes desiccated, e.g., freeze-dried, compositions comprising an anti-PfRH5 antigen-binding protein, e.g., antibody or antigen-binding fragment thereof (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2), or a pharmaceutical composition thereof that includes a pharmaceutically acceptable carrier but substantially lacks water.

In a further embodiment of the invention, a further therapeutic agent that is administered to a subject in association with an anti-PfRH5 antigen-binding protein, e.g., antibody or antigen-binding fragment thereof (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2), disclosed herein is administered to the subject in accordance with the Physicians' Desk Reference 2003 (Thomson Healthcare; 57th edition (Nov. 1, 2002)).

The mode of administration of an antigen-binding protein or composition thereof can vary. Routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal or intra-arterial.

The present invention provides methods for administering an anti-PfRH5 antigen-binding protein, e.g., antibody or antigen-binding fragment thereof (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2) to a subject, comprising introducing the protein or a pharmaceutical composition or combination thereof into the body of the subject. For example, the method comprises piercing the body of the subject with a needle of a syringe and injecting the antigen-binding protein or a pharmaceutical composition or combination thereof into the body of the subject, e.g., into the vein, artery, tumor, muscular tissue or subcutis of the subject.

The present invention provides a vessel (e.g., a plastic or glass vial, e.g., with a cap or a chromatography column, hollow bore needle or a syringe cylinder) comprising any of the anti-PfRH5 antigen-binding proteins, e.g., antibodies or antigen-binding fragments thereof (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2) or a pharmaceutical composition comprising a pharmaceutically acceptable carrier or combination thereof.

The present invention includes combinations including an anti-PfRH5 antigen-binding protein, e.g., antibody or antigen-binding fragment thereof of the present invention (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2), in association with one or more further therapeutic agents. The anti-PfRH5 antigen-binding protein and the further therapeutic agent can be in a single composition or in separate compositions. For example, in an embodiment of the invention, the further therapeutic agent is an anti-parasitic or anti-malarial therapeutic agent. In an embodiment of the invention, the further therapeutic agent is chloroquine, atovaquone and/or proguanil, artemether and/or lumefantrine, mefloquine, quinine, quinidine, doxycycline (optionally in combination with quinine) and/or clindamycin (optionally in combination with quinine). In an embodiment of the invention, the further therapeutic agent is a vaccine such as an anti-malarial vaccine, e.g., RTS,S/AS01 (sold as Mosquirix). Methods for treating or preventing Plasmodium falciparum infection in a subject in need of said treatment or prevention by administering H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2 in association with a further therapeutic agent are part of the present invention.

The present invention includes a combination comprising two or more (e.g., 2, 3 or 4) of the antigen-binding proteins of the present invention (e.g., antibody or antigen-binding protein) in association with one another. For example, if H1H29089P is Ab1; if H1H29094P is Ab2; if H1H29100P is Ab3; if H1H29104P is Ab4; if H1H29106P is Ab5; if H1H29109P is Ab6; if H1H29125P is Ab7; if H1H29127P is Ab8; if H1H29131P is Ab9; if H1H29134P is Ab10; if H1H29138P is Ab11; if H1H29141P is Ab12; if H1H29143P is Ab13; if H1H29146P2 is Ab14; if H1H29147P2 is Ab15; if H1H29149P2 is Ab16; if H1H29151P2 is Ab17; if H1H29163P2 is Ab18; if H1H29166P2 is Ab19; if H1H29171P2 is Ab20; if H1H29179P2 is Ab21; if H1H29183P2 is Ab22; if H1H29187P2 is Ab23; if H1H29192P2 is Ab24; if H1H29196P2 is Ab25; if H1H29198P2 is Ab26; if H1H29207P2 is Ab27; if H1H29214P2 is Ab28; if H1H29215P2 is Ab29 and if H1H29209P is Ab30, then such compositions of the present invention include combinations including the following antigen-binding proteins of the present invention (e.g., antibodies and/or antigen-binding proteins) in association with one another:

| | | | | | | | | | |
|---|---|---|---|---|---|---|---|---|---|
| Ab1 & | Ab1 & | Ab2 & | Ab2 & | Ab3 & | Ab3 & | Ab4 & | Ab5 & | Ab5 & | Ab6 & |
| Ab2 | Ab18 | Ab7 | Ab23 | Ab13 | Ab29 | Ab20 | Ab12 | Ab28 | Ab21 |
| Ab1 & | Ab1 & | Ab2 & | Ab2 & | Ab3 & | Ab4 & | Ab4 & | Ab5 & | Ab5 & | Ab6 & |
| Ab3 | Ab19 | Ab8 | Ab24 | Ab14 | Ab5 | Ab21 | Ab13 | Ab29 | Ab22 |
| Ab1 & | Ab1 & | Ab2 & | Ab2 & | Ab3 & | Ab4 & | Ab4 & | Ab5 & | Ab6 & | Ab6 & |
| Ab4 | Ab20 | Ab9 | Ab25 | Ab15 | Ab6 | Ab22 | Ab14 | Ab7 | Ab23 |
| Ab1 & | Ab1 & | Ab2 & | Ab2 & | Ab3 & | Ab4 & | Ab4 & | Ab5 & | Ab6 & | Ab6 & |
| Ab5 | Ab21 | Ab10 | Ab26 | Ab16 | Ab7 | Ab23 | Ab15 | Ab8 | Ab24 |
| Ab1 & | Ab1 & | Ab2 & | Ab2 & | Ab3 & | Ab4 & | Ab4 & | Ab5 & | Ab6 & | Ab6 & |
| Ab6 | Ab22 | Ab11 | Ab27 | Ab17 | Ab8 | Ab24 | Ab16 | Ab9 | Ab25 |
| Ab1 & | Ab1 & | Ab2 & | Ab2 & | Ab3 & | Ab4 & | Ab4 & | Ab5 & | Ab6 & | Ab6 & |
| Ab7 | Ab23 | Ab12 | Ab28 | Ab18 | Ab9 | Ab25 | Ab17 | Ab10 | Ab26 |
| Ab1 & | Ab1 & | Ab2 & | Ab2 & | Ab3 & | Ab4 & | Ab4 & | Ab5 & | Ab6 & | Ab6 & |
| Ab8 | Ab24 | Ab13 | Ab29 | Ab19 | Ab10 | Ab26 | Ab18 | Ab11 | Ab27 |
| Ab1 & | Ab1 & | Ab2 & | Ab3 & | Ab3 & | Ab4 & | Ab4 & | Ab5 & | Ab6 & | Ab6 & |
| Ab9 | Ab25 | Ab14 | Ab4 | Ab20 | Ab11 | Ab27 | Ab19 | Ab12 | Ab28 |
| Ab1 & | Ab1 & | Ab2 & | Ab3 & | Ab3 & | Ab4 & | Ab4 & | Ab5 & | Ab6 & | Ab6 & |
| Ab10 | Ab26 | Ab15 | Ab5 | Ab21 | Ab12 | Ab28 | Ab20 | Ab13 | Ab29 |
| Ab1 & | Ab1 & | Ab2 & | Ab3 & | Ab3 & | Ab4 & | Ab4 & | Ab5 & | Ab6 & | Ab7 & |
| Ab11 | Ab27 | Ab16 | Ab6 | Ab22 | Ab13 | Ab29 | Ab21 | Ab14 | Ab8 |
| Ab1 & | Ab1 & | Ab2 & | Ab3 & | Ab3 & | Ab4 & | Ab5 & | Ab5 & | Ab6 & | Ab7 & |
| Ab12 | Ab28 | Ab17 | Ab7 | Ab23 | Ab14 | Ab6 | Ab22 | Ab15 | Ab9 |
| Ab1 & | Ab1 & | Ab2 & | Ab3 & | Ab3 & | Ab4 & | Ab5 & | Ab5 & | Ab6 & | Ab7 & |
| Ab13 | Ab29 | Ab18 | Ab8 | Ab24 | Ab15 | Ab7 | Ab23 | Ab16 | Ab10 |
| Ab1 & | Ab2 & | Ab2 & | Ab3 & | Ab3 & | Ab4 & | Ab5 & | Ab5 & | Ab6 & | Ab7 & |
| Ab14 | Ab3 | Ab19 | Ab9 | Ab25 | Ab16 | Ab8 | Ab24 | Ab17 | Ab11 |
| Ab1 & | Ab2 & | Ab2 & | Ab3 & | Ab3 & | Ab4 & | Ab5 & | Ab5 & | Ab6 & | Ab7 & |
| Ab15 | Ab4 | Ab20 | Ab10 | Ab26 | Ab17 | Ab9 | Ab25 | Ab18 | Ab12 |
| Ab1 & | Ab2 & | Ab2 & | Ab3 & | Ab3 & | Ab4 & | Ab5 & | Ab5 & | Ab6 & | Ab7 & |
| Ab16 | Ab5 | Ab21 | Ab11 | Ab27 | Ab18 | Ab10 | Ab26 | Ab19 | Ab13 |
| Ab1 & | Ab2 & | Ab2 & | Ab3 & | Ab3 & | Ab4 & | Ab5 & | Ab5 & | Ab6 & | Ab7 & |
| Ab17 | Ab6 A | Ab22 | Ab12 | Ab28 | Ab19 | Ab11 | Ab27 | Ab20 | Ab14 |
| Ab7 & | Ab8 & | Ab8 & | Ab9 & | Ab10 | Ab10 | Ab11 | Ab12 | Ab13 | Ab13 |
| Ab15 | Ab10 | Ab26 | Ab22 | & | & | & | & | & | & |
| Ab7 & | Ab8 & | Ab8 & | Ab9 & | Ab17 | Ab29 | Ab23 | Ab18 | Ab14 | Ab26 |
| Ab16 | Ab11 | Ab27 | Ab23 | Ab10 | Ab11 | Ab11 | Ab12 | Ab13 | Ab13 |
| Ab7 & | Ab8 & | Ab8 & | Ab9 & | & | & | & | & | & | & |
| Ab17 | Ab12 | Ab28 | Ab24 | Ab18 | Ab12 A | Ab24 | Ab19 | Ab15 | Ab27 |
| Ab7 & | Ab8 & | Ab8 & | Ab9 & | Ab10 | Ab11 | Ab11 | Ab12 | Ab13 | Ab13 |
| Ab18 | Ab13 | Ab29 | Ab25 | & | & | & | & | & | & |
| Ab7 & | Ab8 & | Ab9 & | Ab9 & | Ab19 | Ab13 | Ab25 | Ab20 | Ab16 | Ab28 |
| Ab19 | Ab14 | Ab10 | Ab26 | Ab10 | Ab11 | Ab11 | Ab12 | Ab13 | Ab13 |
| Ab7 & | Ab8 & | Ab9 & | Ab9 & | & | & | & | & | & | & |
| Ab20 | Ab15 | Ab11 | Ab27 | Ab20 | Ab14 | Ab26 | Ab21 | Ab17 | Ab29 |
| Ab7 & | Ab8 & | Ab9 & | Ab9 & | Ab10 | Ab11 | Ab11 | Ab12 | Ab13 | Ab14 |
| Ab21 | Ab16 | Ab12 | Ab28 | & | & | & | & | & | & |
| Ab7 & | Ab8 & | Ab9 & | Ab9 & | Ab21 | Ab15 | Ab27 | Ab22 | Ab18 | Ab15 |
| Ab22 | Ab17 | Ab13 | Ab29 | Ab10 | Ab11 | Ab11 | Ab12 | Ab13 | Ab14 |
| Ab7 & | Ab8 & | Ab9 & | Ab10 | & | & | & | & | & | & |
| Ab23 | Ab18 | Ab14 | & | Ab22 | Ab16 | Ab28 | Ab23 | Ab19 | Ab16 |
| Ab7 & | Ab8 & | Ab9 & | Ab11 | Ab10 | Ab11 | Ab11 | Ab12 | Ab13 | Ab14 |
| Ab24 | Ab19 | Ab15 | Ab10 | & | & | & | & | & | & |
| Ab7 & | Ab8 & | Ab9 & | & | Ab23 | Ab17 | Ab29 | Ab24 | Ab20 | Ab17 |
| Ab25 | Ab20 | Ab16 | Ab12 | Ab10 | Ab11 | Ab12 | Ab12 | Ab13 | Ab14 |
| Ab7 & | Ab8 & | Ab9 & | Ab10 | & | & | & | & | & | & |
| Ab26 | Ab21 | Ab17 | & | Ab24 | Ab18 | Ab13 | Ab25 | Ab21 | Ab18 |
| Ab7 & | Ab8 & | Ab9 & | Ab13 | Ab10 | Ab11 | Ab12 | Ab12 | Ab13 | Ab14 |
| Ab27 | Ab22 | Ab18 | Ab10 | & | & | & | & | & | & |
| Ab7 & | Ab8 & | Ab9 & | & | Ab25 | Ab19 | Ab14 | Ab26 | Ab22 | Ab19 |
| Ab28 | Ab23 | Ab19 | Ab14 | Ab10 | Ab11 | Ab12 | Ab12 | Ab13 | Ab14 |
| Ab7 & | Ab8 & | Ab9 & | Ab10 | & | & | & | & | & | & |
| Ab29 | Ab24 | Ab20 | & | Ab26 | Ab20 | Ab15 | Ab27 | Ab23 | Ab20 |
| Ab8 & | Ab8 & | Ab9 & | Ab15 | Ab10 | Ab11 | Ab12 | Ab12 | Ab13 | Ab14 |
| Ab9 | Ab25 | Ab21 | Ab10 | & | & | & | & | & | & |
| Ab14 | Ab15 | Ab16 | & | Ab27 | Ab21 | Ab16 | Ab28 | Ab24 | Ab21 |
| & | & | & | Ab16 | Ab10 | Ab11 | Ab12 | Ab12 | Ab13 | Ab14 |
| Ab23 | Ab21 | Ab20 | Ab17 | & | & | & | & | & | & |
| Ab14 | Ab15 | Ab16 | & | Ab28 | Ab22 | Ab17 | Ab29 | Ab25 | Ab22 |
| & | & | & | Ab20 | Ab18 | Ab19 | Ab20 | Ab22 | Ab23 | Ab26 |
| Ab24 | Ab22 | Ab21 | Ab17 | & | & | & | & | & | & |
| Ab14 | Ab15 | Ab16 | & | Ab21 | Ab23 | Ab26 | Ab23 | Ab29 | Ab29 |
| & | & | & | Ab21 | Ab18 | Ab19 | Ab20 | Ab22 | Ab24 | Ab27 |
| Ab25 | Ab23 | Ab22 | Ab17 | & | & | & | & | & | & |
| Ab14 | Ab15 | Ab16 | & | Ab22 | Ab24 | Ab27 | Ab24 | Ab25 | Ab28 |
| & | & | & | Ab22 | Ab18 | Ab19 | Ab20 | Ab22 | Ab24 | Ab27 |
| Ab26 | Ab24 | Ab23 | Ab17 | & | & | & | & | & | & |
| Ab14 | Ab15 | Ab16 | & | Ab23 | Ab25 | Ab28 | Ab25 | Ab26 | Ab29 |
| & | & | & | Ab23 | Ab18 | Ab19 | Ab20 | Ab22 | Ab24 | Ab28 |
| Ab27 | Ab25 | Ab24 | Ab17 | & | & | & | & | & | & |
| Ab14 | Ab15 | Ab16 | & | Ab24 | Ab26 | Ab29 | Ab26 | Ab27 | Ab29 |
| & | & | & | Ab24 | Ab18 | Ab19 | Ab21 | Ab22 | Ab24 | Ab1 & |
| Ab28 | Ab26 | Ab25 | Ab17 | & | & | & | & | & | Ab30 |
| Ab14 | Ab15 | Ab16 | & | Ab25 | Ab27 | Ab22 | Ab27 | Ab28 | Ab2 & |
| & | & | & | Ab25 | Ab18 | Ab19 | Ab21 | Ab22 | Ab24 | Ab30 |
| Ab29 | Ab27 | Ab26 | Ab17 | & | & | & | & | & | Ab3 & |
| Ab15 | Ab15 | Ab16 | & | Ab26 | Ab28 | Ab23 | Ab28 | Ab29 | Ab30 |
| & | & | & | Ab26 | Ab18 | Ab19 | Ab21 | Ab22 | Ab25 | Ab4 & |
| Ab16 | Ab28 | Ab27 | Ab17 | & | & | & | & | & | Ab30 |
| Ab15 | Ab15 | Ab16 | & | Ab27 | Ab29 | Ab24 | Ab29 | Ab26 | Ab5 & |
| & | & | & | Ab27 | Ab18 | Ab20 | Ab21 | Ab23 | Ab25 | Ab30 |
| Ab17 | Ab29 | Ab28 | Ab17 | & | & | & | & | & | Ab6 & |
| Ab15 | Ab16 | Ab16 | & | Ab28 | Ab21 | Ab25 | Ab24 | Ab27 | Ab30 |
| & | & | & | Ab28 | Ab18 | Ab20 | Ab21 | Ab23 | Ab25 | Ab7 & |
| Ab18 | Ab17 | Ab29 | Ab17 | & | & | & | & | & | Ab30 |
| Ab15 | Ab16 | Ab17 | & | Ab29 | Ab22 | Ab26 | Ab25 | Ab28 | Ab8 & |
| & | & | & | Ab29 | Ab19 | Ab20 | Ab21 | Ab23 | Ab25 | Ab30 |
| Ab19 | Ab18 | Ab18 | Ab18 | & | & | & | & | & | Ab9 & |
| Ab15 | Ab16 | Ab17 | & | Ab20 | Ab23 | Ab27 | Ab26 | Ab29 | Ab30 |
| & | & | & | Ab19 | Ab19 | Ab20 | Ab21 | Ab23 | Ab26 | Ab10 |
| Ab20 | Ab19 | Ab19 | Ab18 | & | & | & | & | & | & |
| Ab11 | Ab13 | Ab15 | & | Ab21 | Ab24 | Ab28 | Ab27 | Ab27 | Ab30 |
| & | & | & | Ab20 | Ab19 | Ab20 | Ab21 | Ab23 | Ab26 | Ab29 |
| Ab30 | Ab30 | Ab30 | Ab17 | & | & | & | & | & | & |
| Ab12 | Ab14 | Ab16 | & | Ab22 | Ab25 | Ab29 | Ab28 | Ab28 | Ab30 |
| & | & | & | Ab30 | Ab19 | Ab21 | Ab23 | Ab25 | Ab27 | |
| Ab30 | Ab30 | Ab30 | Ab18 | & | & | & | & | & | |
| | | | & | Ab30 | Ab30 | Ab30 | Ab30 | Ab30 | |
| | | | Ab30 | Ab20 | Ab22 | Ab24 | Ab26 | Ab28 | |
| | | | | & | & | & | & | & | |
| | | | | Ab30 | Ab30 | Ab30 | Ab30 | Ab30 | |

In an embodiment of the invention, the composition comprises two or more non-competing antigen-binding proteins. Cross-competition between anti-PfRH5 antibodies of the present invention is summarized below in Table 6-1.

The term “in association with” indicates that components, an anti-PfRH5 antigen-binding protein, e.g., antibody or antigen-binding fragment thereof of the present invention, along with another agent such as chloroquine, can be formulated into a single composition, e.g., for simultaneous delivery, or formulated separately into two or more compositions (e.g., a kit including each component). Each component can be administered to a subject at a different time than when the other component is administered; for example, each administration may be given non-simultaneously (e.g., separately or sequentially) at intervals over a given period of time. Moreover, the separate components may be administered to a subject by the same or by a different route.

### Testing and Diagnosis

Early diagnosis of malaria is helpful to obtaining a positive clinical outcome. The present invention provides methods for treating Plasmodium falciparum infection (e.g., malaria), in a subject, comprising diagnosing the infection in the subject and, if the subject is diagnosed as having the infection, administering a therapeutically effective amount of the anti-PfRH5 antigen-binding protein, e.g., antibody or antigen-binding fragment, to the subject. See e.g., Moody, Rapid Diagnostic Tests for Malaria Parasites, Clinical Microbiology Reviews 15 (1): 66-78 (2002).

Plasmodium falciparum infection can be diagnosed microscopically (e.g., fluorescence microscopy). For example, Plasmodium falciparum can be identified by examining, under the microscope, a drop of a subject's blood, e.g., spread out as a “blood smear” on a microscope slide (e.g., thick blood film or thin blood film). Prior to examination, the specimen can be stained (e.g., with Giemsa stain). The present invention includes methods for treatment of Plasmodium falciparum infection (as discussed herein) wherein the infection is diagnosed microscopically.

In an embodiment of the invention, the presence of Plasmodium falciparum in a sample from the subject is detected by detecting Plasmodium falciparum lactate dehydrogenase. If LDH is detected in a test sample above that of a control sample of a known uninfected sample, then the test sample is determined to contain Plasmodium falciparum. See e.g., Miura, H. Zhou, A. Diouf, SE. Moretz, MP. Fay, LH. Miller, LB. Martin, MA. Pierce, RD. Ellis, GED. Mullen, CA. Long. Anti-Apical-Membrane-Antigen-1 antibody is more effective than anti-42-kilodalton-Merozoite-Surface-Protein-1 antibody in inhibiting Plasmodium falciparum growth, as determined by the in vitro growth inhibition assay. Clin Vaccine Immunol. 16, 963-968 (2009). PMID: PMC2708396.

The presence of Plasmodium falciparum nucleic acids can also be detected, e.g., using polymerase chain reaction (PCR) to detect, for example, the small-subunit 18S rRNA and/or circumsporozoite (CS) genes.

Plasmodium falciparum infection can also be diagnosed by detection of the parasite's antigens in the body of a subject. For example, in an embodiment of the invention, the antigen is detected immunogenically, e.g., using a rapid diagnostic test. See e.g., Van der Palen et al. Test characteristics of two rapid antigen detection tests (SD FK50 and SD FK60) for the diagnosis of malaria in returned travelers, Malaria Journal 8:90 (2009); or U.S. Pat. No. 5,712,170.

The anti-PfRH5 antigen-binding proteins, e.g., antibodies or antigen-binding fragments thereof of the present invention (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2), may be used to detect and/or measure PfRH5 (e.g., a Plasmodium falciparum cell which includes the PfRH5 protein) in a sample (e.g., a bodily fluid such as blood). Exemplary assays for PfRH5 may include, e.g., contacting the sample with an anti-PfRH5 antigen-binding protein of the invention, wherein, for example, the anti-PfRH5 antigen-binding protein is labeled with a detectable label or reporter molecule. If the anti-PfRH5 antigen-binding protein complexed with PfRH5 is detected, then this indicates the presence of PfRH5 in the sample and/or the presence of Plasmodium falciparum in the sample and in the body of the subject.

For example, the present invention includes methods for detecting PfRH5 polypeptide or a cell including such a polypeptide (e.g., Plasmodium falciparum) using a lateral flow ‘immuno-chromatographic’ antigen-detection test. Such lateral flow tests rely on the capture of detectably labeled antigen-binding proteins (e.g., antibodies and antigen-binding fragments thereof) to produce a visible band on a strip of substrate. With Plasmodium falciparum malaria diagnostic tests, the labeled antigen-binding protein first binds to the parasite antigen, PfRH5, and the resultant complex is captured on the strip by a band of bound antigen-binding protein, forming a visible line (test line). A control line gives information on the integrity of the antibody-label conjugate, but does not confirm the ability to detect parasite antigen.

The present invention provides a lateral flow test strip for detecting the presence of PfRH5 (e.g., a Plasmodium falciparum cell) in a sample comprising a substrate (e.g., nitrocellulose) which includes the following regions arranged laterally across the substrate:
(i) a spot for introduction of a sample (sample zone);
(ii) a conjugate pad which includes a first anti-PfRH5 antigen-binding protein (e.g., H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; or H1H29215P2) which is detectably labeled, e.g., with a dye, but not immobilized to the substrate;
(iii) a test line containing a second anti-PfRH5 antigen-binding protein which is not detectably labeled and does not significantly compete with the first anti-PfRH5 antigen-binding protein for binding to PfRH5, but is immobilized to the substrate; and
(iv) a control line containing a secondary antigen-binding protein, immobilized to the substrate, which binds to the first anti-PfRH5 antigen-binding protein (e.g., an antibody or antigen-binding fragment or protein-A).

In an embodiment of the invention:
(i) once a liquid-containing sample is placed on the sample pad, the sample (and any PfRH5 polypeptide contained therein) diffuses, by capillary action, across the substrate, onto the conjugation pad, then into the test line and then into the control line;
(ii) PfRH5 polypeptide which reaches the conjugate pad forms a complex with the detectably labeled first anti-PfRH5 antigen-binding protein, and the complex further diffuses into the test line wherein a further complex is formed with the second anti-PfRH5 antigen-binding protein (forming a triple complex wherein PfRH5 is bound by said first and second antigen-binding proteins and immobilized within the test line);
(iii) any excess first anti-PfRH5 antigen-binding protein which does not immobilize at the test line continues to diffuse into the control line and binds to the secondary antigen-binding protein, thereby becoming immobilized in the control line; and/or
(iv) a positive test, indicating the presence of PfRH5 in the sample, is indicated by the presence of the detectable label in the test line; a negative test, indicating the lack of detectable levels of PfRH5 in the sample, is indicated by the absence of the detectable label in the test line; and detectable label in the control line indicates that the test system is operating properly.

In an embodiment of the invention, the detectable label is a dye (e.g., indigo blue), an enzyme, a ferritin, a fluorescent or colored microparticle/bead or nanoparticle/bead or a colloid metal (e.g., gold, selenium dye (e.g., in a liposome) or silver, e.g., a colloidal particle thereof). In an embodiment of the invention, the detectable label is visually detectable.

In an embodiment of the invention, the substrate is an insoluble material capable of supporting fluid flow, e.g., glass fiber filter paper; natural polymeric materials, cellulose-based materials, filter paper, chromatographic paper, nitrocellulose, cellulose acetate, poly (vinyl chloride), polyacrylamide or crosslinked dextran.

The present invention also provides a method for determining if a sample (e.g., bodily fluid such as, for example, blood) contains PfRH5 (e.g., a cell containing PfRH5 such as Plasmodium falciparum) comprising contacting the sample zone of a lateral flow device, as set forth herein, with the sample, waiting for capillary flow to carry the sample across the substrate to the control line, optionally waiting for a period of time, and observing the test and control lines, wherein the presence of the detectable label in the test line indicates that the sample contained PfRH5 and the presence of the detectable label in the control line indicates that the lateral flow test strip is functioning correctly. The absence of detectable label in the test line, with the presence of detectable label in the control line indicates the absence of PfRH5 from the sample. In an embodiment of the invention, the method further comprises treating the subject, from whom the sample was taken, with a therapeutically effective amount of anti-PfRH5 antigen-binding protein (e.g., antibody or antigen-binding fragment) if the test indicates the presence of PfRH5 in the sample and the flow test strip is functioning correctly.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Antibodies That Bind to PfRH5

Human antibodies to P. falciparum RH5 (PfRH5) were generated in a VELOCIMMUNE® mouse comprising DNA encoding human immunoglobulin heavy and kappa light chain variable regions. The mice were immunized with recombinant RH5 protein (PfRH5ΔNL). Some mice were immunized with recombinant PfRH5 protein (PfRH5ΔNL.6his) followed by a booster of P. falciparum merozoites isolated from strain 3D7. The antibody immune response was monitored by a PfRH5-specific immunoassay. When a desired immune response was achieved, splenocytes were harvested and antibodies isolated directly from antigen-positive mouse B cells without fusion to myeloma cells, as described in U.S. Pat. No. 7,582,298, herein specifically incorporated by reference in its entirety. Using this method, several fully human anti-PfRH5 antibodies (i.e., antibodies possessing human variable domains and human constant domains) were obtained; exemplary antibodies generated in this manner were designated as H1H29089P; H1H29094P; H1H29100P; H1H29104P; H1H29106P; H1H29109P; H1H29125P; H1H29127P; H1H29131P; H1H29134P; H1H29138P; H1H29141P; H1H29143P; H1H29146P2; H1H29147P2; H1H29149P2; H1H29151P2; H1H29163P2; H1H29166P2; H1H29171P2; H1H29179P2; H1H29183P2; H1H29187P2; H1H29192P2; H1H29196P2; H1H29198P2; H1H29207P2; H1H29209P2; H1H29214P2; and H1H29215P2.

The biological properties of the exemplary antibodies generated in accordance with the methods of this Example are described in detail in the Examples set forth below. Sequences of the antibody immunoglobulin chains are set forth below.

TABLE 1-1
Immunoglobulin chain sequences of the present invention*

| Antibody | | V_{H} | | CDR-H1 | | CDR-H2 | | CDR-H3 | | V_{L} | | CDR-L1 | | CDR-L2 | | CDR-L3 | |
|---|---|---|---|---|---|---|---|---|---|---|---|---|---|---|---|---|---|
| # | Name | DNA | PEP | DNA | PEP | DNA | PEP | DNA | PEP | DNA | PEP | DNA | PEP | DNA | PEP | DNA | PEP |
| 1 | H1H29089P | 1 | 2 | 3 | 4 | 5 | 6 | 7 | 8 | 9 | 10 | 11 1 | 12 | 13 | 14 | 15 | 16 |
| 2 | H1H29094P | 17 | 18 | 19 | 20 | 21 | 22 | 23 | 24 | 25 | 26 | 27 | 28 | 29 | 30 | 31 | 32 |
| 3 | H1H29100P | 33 | 34 | 35 | 36 | 37 | 38 | 39 | 40 | 41 | 42 | 43 | 44 | 45 | 46 | 47 | 48 |
| 4 | H1H29104P | 49 | 50 | 51 | 52 | 53 | 54 | 55 | 56 | 57 | 58 | 59 | 60 | 6 | 62 | 63 | 64 |
| 5 | H1H29106P | 65 | 66 | 67 | 68 | 69 | 70 | 71 | 72 | 73 | 74 | 75 | 76 | 77 | 78 | 79 | 80 |
| 6 | H1H29109P | 81 | 82 | 83 | 84 | 85 | 86 | 87 | 88 | 89 | 90 | 91 | 92 | 93 | 94 | 95 | 96 |
| 7 | H1H29125P | 97 | 98 | 99 | 100 | 101 | 102 | 103 | 104 | 105 | 106 | 107 | 108 | 109 | 110 | 111 | 112 |
| 8 | H1H29127P | 113 | 114 | 115 | 116 | 117 | 118 | 119 | 120 | 121 | 122 | 123 | 124 | 125 | 126 | 127 | 128 |
| 9 | H1H29131P | 129 | 130 | 131 | 132 | 133 | 134 | 135 | 136 | 137 | 138 | 139 | 140 | 141 | 142 | 143 | 144 |
| 10 | H1H29134P | 145 | 146 | 147 | 148 | 149 | 150 | 151 | 152 | 153 | 154 | 155 | 156 | 157 | 158 | 159 | 160 |
| 11 | H1H29138P | 161 | 162 | 163 | 164 | 165 | 166 | 167 | 168 | 169 | 170 | 171 | 172 | 173 | 174 | 175 | 176 |
| 12 | H1H29141P | 177 | 178 | 179 | 180 | 181 | 182 | 183 | 184 | 185 | 186 | 187 | 188 | 189 | 190 | 191 | 192 |
| 13 | H1H29143P | 193 | 194 | 195 | 196 | 197 | 198 | 199 | 200 | 201 | 202 | 203 | 204 | 205 | 206 | 207 | 208 |
| 14 | H1H29146P2 | 209 | 210 | 211 | 212 | 213 | 214 | 215 | 216 | 217 | 218 | 219 | 220 | 221 | 222 | 223 | 224 |
| 15 | H1H29147P2 | 225 | 226 | 227 | 228 | 229 | 230 | 231 | 232 | 217 | 218 | 219 | 220 | 221 | 222 | 223 | 224 |
| 16 | H1H29149P2 | 233 | 234 | 235 | 236 | 237 | 238 | 239 | 240 | 217 | 218 | 219 | 220 | 221 | 222 | 223 | 224 |
| 17 | H1H29151P2 | 241 | 242 | 243 | 244 | 245 | 246 | 247 | 248 | 217 | 218 | 219 | 220 | 221 | 222 | 223 | 224 |
| 18 | H1H29163P2 | 249 | 250 | 251 | 252 | 253 | 254 | 255 | 256 | 217 | 218 | 219 | 220 | 221 | 222 | 223 | 224 |
| 19 | H1H29166P2 | 257 | 258 | 259 | 260 | 261 | 262 | 263 | 264 | 217 | 218 | 219 | 220 | 221 | 222 | 223 | 224 |
| 20 | H1H29171P2 | 265 | 266 | 267 | 268 | 269 | 270 | 271 | 272 | 217 | 218 | 219 | 220 | 221 | 222 | 223 | 224 |
| 21 | H1H29179P2 | 273 | 274 | 275 | 276 | 277 | 278 | 279 | 280 | 217 | 218 | 219 | 220 | 221 | 222 | 223 | 224 |
| 22 | H1H29183P2 | 281 | 282 | 283 | 284 | 285 | 286 | 287 | 288 | 217 | 218 | 219 | 220 | 221 | 222 | 223 | 224 |
| 23 | H1H29187P2 | 289 | 290 | 291 | 292 | 293 | 294 | 295 | 296 | 217 | 218 | 219 | 220 | 221 | 222 | 223 | 224 |
| 24 | H1H29192P2 | 297 | 298 | 299 | 300 | 301 | 302 | 303 | 304 | 305 | 306 | 307 | 308 | 309 | 310 | 311 | 312 |
| 25 | H1H29196P2 | 313 | 314 | 315 | 316 | 317 | 318 | 319 | 320 | 305 | 306 | 307 | 308 | 309 | 310 | 311 | 312 |
| 26 | H1H29198P2 | 321 | 322 | 323 | 324 | 325 | 326 | 327 | 328 | 305 | 306 | 307 | 308 | 309 | 310 | 311 | 312 |
| 27 | H1H29207P2 | 329 | 330 | 331 | 332 | 333 | 334 | 335 | 336 | 305 | 306 | 307 | 308 | 309 | 310 | 311 | 312 |
| 28 | H1H29214P2 | 337 | 338 | 339 | 340 | 341 | 342 | 343 | 344 | 305 | 306 | 307 | 308 | 309 | 310 | 311 | 312 |
| 29 | H1H29215P2 | 345 | 346 | 347 | 348 | 349 | 350 | 351 | 352 | 305 | 306 | 307 | 308 | 309 | 310 | 311 | 312 |
| 30 | H1H29209P2 | 353 | 354 | 355 | 356 | 357 | 358 | 359 | 360 | 305 | 306 | 307 | 308 | 309 | 310 | 311 | 312 |

*Numbers corresponding to V_{H}, CDR-H1, CDR-H2, CDR-H3, V_{L}, CDR-L1, CDR-L2 and CDR-L3 refer to SEQ ID NOs set forth herein.
“PEP” refers to an amino acid sequence;
“DNA” refers to a nucleotide sequence.

TABLE 1-1
Immunoglobulin chain sequences of the present invention*

| Antibody | | V_{H} | | CDR-H1 | | CDR-H2 | | CDR-H3 | | V_{L} | | CDR-L1 | | CDR-L2 | | CDR-L3 | |
|---|---|---|---|---|---|---|---|---|---|---|---|---|---|---|---|---|---|
| # | Name | DNA | PEP | DNA | PEP | DNA | PEP | DNA | PEP | DNA | PEP | DNA | PEP | DNA | PEP | DNA | PEP |
| 1 | H1H29089P | 1 | 2 | 3 | 4 | 5 | 6 | 7 | 8 | 9 | 10 | 11 1 | 12 | 13 | 14 | 15 | 16 |
| 2 | H1H29094P | 17 | 18 | 19 | 20 | 21 | 22 | 23 | 24 | 25 | 26 | 27 | 28 | 29 | 30 | 31 | 32 |
| 3 | H1H29100P | 33 | 34 | 35 | 36 | 37 | 38 | 39 | 40 | 41 | 42 | 43 | 44 | 45 | 46 | 47 | 48 |
| 4 | H1H29104P | 49 | 50 | 51 | 52 | 53 | 54 | 55 | 56 | 57 | 58 | 59 | 60 | 6 | 62 | 63 | 64 |
| 5 | H1H29106P | 65 | 66 | 67 | 68 | 69 | 70 | 71 | 72 | 73 | 74 | 75 | 76 | 77 | 78 | 79 | 80 |
| 6 | H1H29109P | 81 | 82 | 83 | 84 | 85 | 86 | 87 | 88 | 89 | 90 | 91 | 92 | 93 | 94 | 95 | 96 |
| 7 | H1H29125P | 97 | 98 | 99 | 100 | 101 | 102 | 103 | 104 | 105 | 106 | 107 | 108 | 109 | 110 | 111 | 112 |
| 8 | H1H29127P | 113 | 114 | 115 | 116 | 117 | 118 | 119 | 120 | 121 | 122 | 123 | 124 | 125 | 126 | 127 | 128 |
| 9 | H1H29131P | 129 | 130 | 131 | 132 | 133 | 134 | 135 | 136 | 137 | 138 | 139 | 140 | 141 | 142 | 143 | 144 |
| 10 | H1H29134P | 145 | 146 | 147 | 148 | 149 | 150 | 151 | 152 | 153 | 154 | 155 | 156 | 157 | 158 | 159 | 160 |
| 11 | H1H29138P | 161 | 162 | 163 | 164 | 165 | 166 | 167 | 168 | 169 | 170 | 171 | 172 | 173 | 174 | 175 | 176 |
| 12 | H1H29141P | 177 | 178 | 179 | 180 | 181 | 182 | 183 | 184 | 185 | 186 | 187 | 188 | 189 | 190 | 191 | 192 |
| 13 | H1H29143P | 193 | 194 | 195 | 196 | 197 | 198 | 199 | 200 | 201 | 202 | 203 | 204 | 205 | 206 | 207 | 208 |
| 14 | H1H29146P2 | 209 | 210 | 211 | 212 | 213 | 214 | 215 | 216 | 217 | 218 | 219 | 220 | 221 | 222 | 223 | 224 |
| 15 | H1H29147P2 | 225 | 226 | 227 | 228 | 229 | 230 | 231 | 232 | 217 | 218 | 219 | 220 | 221 | 222 | 223 | 224 |
| 16 | H1H29149P2 | 233 | 234 | 235 | 236 | 237 | 238 | 239 | 240 | 217 | 218 | 219 | 220 | 221 | 222 | 223 | 224 |
| 17 | H1H29151P2 | 241 | 242 | 243 | 244 | 245 | 246 | 247 | 248 | 217 | 218 | 219 | 220 | 221 | 222 | 223 | 224 |
| 18 | H1H29163P2 | 249 | 250 | 251 | 252 | 253 | 254 | 255 | 256 | 217 | 218 | 219 | 220 | 221 | 222 | 223 | 224 |
| 19 | H1H29166P2 | 257 | 258 | 259 | 260 | 261 | 262 | 263 | 264 | 217 | 218 | 219 | 220 | 221 | 222 | 223 | 224 |
| 20 | H1H29171P2 | 265 | 266 | 267 | 268 | 269 | 270 | 271 | 272 | 217 | 218 | 219 | 220 | 221 | 222 | 223 | 224 |
| 21 | H1H29179P2 | 273 | 274 | 275 | 276 | 277 | 278 | 279 | 280 | 217 | 218 | 219 | 220 | 221 | 222 | 223 | 224 |
| 22 | H1H29183P2 | 281 | 282 | 283 | 284 | 285 | 286 | 287 | 288 | 217 | 218 | 219 | 220 | 221 | 222 | 223 | 224 |
| 23 | H1H29187P2 | 289 | 290 | 291 | 292 | 293 | 294 | 295 | 296 | 217 | 218 | 219 | 220 | 221 | 222 | 223 | 224 |
| 24 | H1H29192P2 | 297 | 298 | 299 | 300 | 301 | 302 | 303 | 304 | 305 | 306 | 307 | 308 | 309 | 310 | 311 | 312 |
| 25 | H1H29196P2 | 313 | 314 | 315 | 316 | 317 | 318 | 319 | 320 | 305 | 306 | 307 | 308 | 309 | 310 | 311 | 312 |
| 26 | H1H29198P2 | 321 | 322 | 323 | 324 | 325 | 326 | 327 | 328 | 305 | 306 | 307 | 308 | 309 | 310 | 311 | 312 |
| 27 | H1H29207P2 | 329 | 330 | 331 | 332 | 333 | 334 | 335 | 336 | 305 | 306 | 307 | 308 | 309 | 310 | 311 | 312 |
| 28 | H1H29214P2 | 337 | 338 | 339 | 340 | 341 | 342 | 343 | 344 | 305 | 306 | 307 | 308 | 309 | 310 | 311 | 312 |
| 29 | H1H29215P2 | 345 | 346 | 347 | 348 | 349 | 350 | 351 | 352 | 305 | 306 | 307 | 308 | 309 | 310 | 311 | 312 |
| 30 | H1H29209P2 | 353 | 354 | 355 | 356 | 357 | 358 | 359 | 360 | 305 | 306 | 307 | 308 | 309 | 310 | 311 | 312 |

*Numbers corresponding to V_{H}, CDR-H1, CDR-H2, CDR-H3, V_{L}, CDR-L1, CDR-L2 and CDR-L3 refer to SEQ ID NOs set forth herein.
“PEP” refers to an amino acid sequence;
“DNA” refers to a nucleotide sequence.

The immunoglobulin sequences and their corresponding SEQ ID NOs which are summarized in Table 1-1 are set forth below. The sequences below are also in a Sequence Listing which is incorporated herein by reference.

SEQ ID NO: 1
GAGGTGCAGCTGGTGCAGTCTGGAGCAGAGGTGAAAAAGCCCGGGGAGTCTCTGAAGATCTCCTGTAAGGGTTCTGGATA
CAGCTTTACCAGTTACTGGATCGTCTGGGTGCGCCAGATGCCCGGGAAAGGCCTGGAGTGGATGGGGATCATCTATCCTG
GTGACTCTGATACCAGATACAGCCCGTCCTTCCAAGGCCAGGTCACCATCTCAGCCGACAAGTCCATCAGCACCGCCTAC
CTGCAGTGGAGCAGCCTGAAGGCCTCGGACACCGCCATGTATTACTGTGCGAGACAAGATATAACTGGAACTACGGGGTT
TGACTACTGGGGCCAGGGAACCCTGGTCACCGTCTCCTCA;
SEQ ID NO: 2
EVQLVQSGAEVKKPGESLKISCKGSGYSFTSYWIVWVRQMPGKGLEWMGIIYPGDSDTRYSPSFQGQVTISADKSISTAY
LQWSSLKASDTAMYYCARQDITGTTGFDYWGQGTLVTVSS;
SEQ ID NO: 3
GGA TAC AGC TTT ACC AGT TAC TGG;
SEQ ID NO: 4
G Y S F T S Y W;
SEQ ID NO: 5
ATC TAT CCT GGT GAC TCT GAT ACC;
SEQ ID NO: 6
I Y P G D S D T;
SEQ ID NO: 7
GCG AGA CAA GAT ATA ACT GGA ACT ACG GGG TTT GAC TAC;
SEQ ID NO: 8
A R Q D I T G T T G F D Y;
SEQ ID NO: 9
GACATCCAGATGACCCAGTCTCCATCCTCCCTGTCTGCATCTGTAGGAGACAGAGTCACCATCACTTGCCGGGCAAGTCA
GAGCATTAGGAACTATTTGAATTGGTATCAGCAGAAACCAGGGAAAGCCCCTAAGCTCCTGATCTATGCTGCATCCAGTT
TGCAAAGTGGGGTCCCATCAAGGTTCAGTGGCAGTGGATCTGGGACAGATTTCACTCTCACCATCAGCAGTCTGCAACCT
GAAGATTTTGCAACTTATTTCTGTCAACAGAGTTACAGTACCCCATTCACTTTCGGCCCTGGGACCAAAGTGGATATCAA
ACGA;
SEQ ID NO: 10
DIQMTQSPSSLSASVGDRVTITCRASQSIRNYLNWYQQKPGKAPKLLIYAASSLQSGVPSRFSGSGSGTDFTLTISSLQP
EDFATYFCQQSYSTPFTFGPGTKVDIKR;
SEQ ID NO: 11
CAG AGC ATT AGG AAC TAT;
SEQ ID NO: 12
Q S I R N Y;
SEQ ID NO: 13
GCT GCA TCC;
SEQ ID NO: 14
A A S;
SEQ ID NO: 15
CAA CAG AGT TAC AGT ACC CCA TTC ACT;
SEQ ID NO: 16
Q Q S Y S T P F T;
SEQ ID NO: 17
CAGGTGCAGCTGGTGGAGTCTGGGGGAGACGTGGTCCAGCCTGGGAGGTCCCTGCGACTCTCCTGTTCAGGCACTGGATT
CACCTTCAGTAGCTATGCCATGCACTGGGTCCGCCAGGCTCCAGGCAAGGGACTGGAATGGGTGGCACTTATATCATATG
ATGGAAGTAATAAATATTATGGAGACTCCGTGAAGGGCCGATTCACCGTCTCCAGAGACAATTCCAAGAACACGCTGTCT
CTGCAAATGAACAGCCTGAAAACTGAGGACACGGCGATATATTACTGTGCGAAAGAGAGGCTTTTTGGAGTGGTCTCTTA
TTACGGTATGGACGTCTGGGGCCAAGGGACCACGGTCACCGTCTCCTCA;
SEQ ID NO: 18
QVQLVESGGDVVQPGRSLRLSCSGTGFTFSSYAMHWVRQAPGKGLEWVALISYDGSNKYYGDSVKGRFTVSRDNSKNTLS
LQMNSLKTEDTAIYYCAKERLFGVVSYYGMDVWGQGTTVTVSS;
SEQ ID NO: 19
GGA TTC ACC TTC AGT AGC TAT GCC;
SEQ ID NO: 20
G F T F S S Y A;
SEQ ID NO: 21
ATA TCA TAT GAT GGA AGT AAT AAA;
SEQ ID NO: 22
I S Y D G S N K;
SEQ ID NO: 23
GCG AAA GAG AGG CTT TTT GGA GTG GTC TCT TAT TAC GGT ATG GAC GTC;
SEQ ID NO: 24
A K E R L F G V V S Y Y G M D V;
SEQ ID NO: 25
GACATCCAGATGACCCAGTCTCCATCCTCCCTGTCTGCATCTGTAGGAGACAGAGTCACCATCACTTGCCAGGCGAGTCA
GGACATTAATAGGGATCTAAATTGGTATCAGCAGAAATCAGGGAAAGGCCCCAAACTCCTGATCTACGATGCATCCAATT
TGGAAACAGGGGTCCCATCAAGGTTCAGTGGAAATAGATTTGGGACAGATTTTACTTTCACCATCAGCAGACTGCAGCCT
GAAGATATTGCAACATATTTCTGTCAACAGTATAAAAATCTCCCGTACACTTTTGGCCAGGGGACCAAGCTGGAGATCAA
ACGA;
SEQ ID NO: 26
DIQMTQSPSSLSASVGDRVTITCQASQDINRDLNWYQQKSGKGPKLLIYDASNLETGVPSRFSGNRFGTDFTFTISRLQP
EDIATYFCQQYKNLPYTFGQGTKLEIKR;
SEQ ID NO: 27
CAG GAC ATT AAT AGG GAT;
SEQ ID NO: 28
Q D I N R D;
SEQ ID NO: 29
GAT GCA TCC;
SEQ ID NO: 30
D A S;
SEQ ID NO: 31
CAA CAG TAT AAA AAT CTC CCG TAC ACT;
SEQ ID NO: 32
Q Q Y K N L P Y T;
SEQ ID NO: 33
CAGCTGCAGCTGCAGGAGTCGGGCCCAGGACTGGTGAAGCCTTCGGAGACCCTGTCCCTCACCTGCACTGTCTCTGGTGG
CTCCATCAGCAGTAGTAGTTACTACTGGGGCTGGATCCGCCAGCCCCCAGGGAAGGGCCTGGAGTGGATTGGGATTATCT
ATTATAGTGGGAGCACCTACTACAACCCGTCCCTCAAGAGTCGAGTCACCATTTCCGTAGACACGTCCAAGAACCAGTTC
TCCCTGAAGCTGAGCTCTGTGACCGCCGCAGACACGGCTGTGTATTACTGTGCGAGACAGGACAGGGAGGCCCTCTTTGA
CTACTGGGGCCAGGGAACCCTGGTCACCGTCTCCTCA;
SEQ ID NO: 34
QLQLQESGPGLVKPSETLSLTCTVSGGSISSSSYYWGWIRQPPGKGLEWIGIIYYSGSTYYNPSLKSRVTISVDTSKNQF
SLKLSSVTAADTAVYYCARQDREALFDYWGQGTLVTVSS;
SEQ ID NO: 35
GGT GGC TCC ATC AGC AGT AGT AGT TAC TAC;
SEQ ID NO: 36
G G S I S S S S Y Y;
SEQ ID NO: 37
ATC TAT TAT AGT GGG AGC ACC;
SEQ ID NO: 38
I Y Y S G S T;
SEQ ID NO: 39
GCG AGA CAG GAC AGG GAG GCC CTC TTT GAC TAC;
SEQ ID NO: 40
A R Q D R E A L F D Y;
SEQ ID NO: 41
GAAATTGTGCTGACTCAGTCTCCAGACTTTCAGTCTGTGACTCCAAAGGAGAAAGTCACCATCACCTGCCGGGCCAGTCA
GCGCATTGGTAGTAGCTTACACTGGTACCAGCAGAAACCAGATCAGTCTCCAAAGCTCCTCATCAAGTATGCTTCCCAGT
CCTTCTCAGGGGTCCCCTCGAGGTTCAGTGGCAGTGGATCTGGGACAGATTTCACCCTCACCATCAATAGCCTGGAAGCT
GAAGATGCTGCAACGTATTACTGTCATCAGAGTAGTACTTTACCCACCTTCGGCCAAGGGACACGACTGGAGATTAAACG
A;
SEQ ID NO: 42
EIVLTQSPDFQSVTPKEKVTITCRASQRIGSSLHWYQQKPDQSPKLLIKYASQSFSGVPSRFSGSGSGTDFTLTINSLEA
EDAATYYCHQSSTLPTFGQGTRLEIKR;
SEQ ID NO: 43
CAG CGC ATT GGT AGT AGC;
SEQ ID NO: 44
Q R I G S S;
SEQ ID NO: 45
TAT GCT TCC;
SEQ ID NO: 46
Y A S;
SEQ ID NO: 47
CAT CAG AGT AGT ACT TTA CCC ACC;
SEQ ID NO: 48
H Q S S T L P T;
SEQ ID NO: 49
GAAGTGCAGCTGGTGGAGTCTGGGGGAGGCCTGGTACAGCCTGGCAGGTCCCTGAGACTCTCCTGTGCAGCCTCTGGATT
CAGGTTTGACGATTATGCCATGCACTGGGTCCGACAAGCTCCAGGGAAGGGCCTGGAATGGGTCTCAGGTATTAATTGGA
ATAGTGGTGGCAAAGGCTATGCGGACTCTGTGCAGGGCCGATTCACCATCTCCAGAGACAACGCCAAGAACTCCCTTTAT
CTGCAAATGAACAGTCTGAGAACTGAGGACACGGCCTTGTATTATTGTGCAAAAGATAGGGGTATAGCAGCTCGTCTTCT
CTCTCGTGATGCTTTTGATATGTGGGGCCAAGGGACAATGGTCACCGTCTCTTCA;
SEQ ID NO: 50
EVQLVESGGGLVQPGRSLRLSCAASGFRFDDYAMHWVRQAPGKGLEWVSGINWNSGGKGYADSVQGRFTISRDNAKNSLY
LQMNSLRTEDTALYYCAKDRGIAARLLSRDAFDMWGQGTMVTVSS;
SEQ ID NO: 51
GGA TTC AGG TTT GAC GAT TAT GCC;
SEQ ID NO: 52
G F R F D D Y A;
SEQ ID NO: 53
ATT AAT TGG AAT AGT GGT GGC AAA;
SEQ ID NO: 54
I N W N S G G K;
SEQ ID NO: 55
GCA AAA GAT AGG GGT ATA GCA GCT CGT CTT CTC TCT CGT GAT GCT TTT GAT ATG;
SEQ ID NO: 56
A K D R G I A A R L L S R D A F D M;
SEQ ID NO: 57
GACATCCAGTTGACCCAGTCTCCATCCTTCCTGTCTGCATCTGTAGGAGACAGAGTCACCATCACTTGCTGGGCCAGTCA
GGACGTTAGCAGTTATTTAGCCTGGTATCAGCAAAAACCAGGGAAATCCCCTAAGCTCCTAATCTTTGCTGCATCCACTT
TGCAAGGTGGGATCCCATCAAGGTTCAGCGGCAGTGGATCTGGGACAGAATTCACTCTCACAATCAGCAGCCTGCGGCCT
GAAGATTTTGCAACTTATTACTGTCAACACCTTAATACTTACCCGTACACTTTTGGCCAGGGGACCAAGCTGGAGATCAA
ACGA;
SEQ ID NO: 58
DIQLTQSPSFLSASVGDRVTITCWASQDVSSYLAWYQQKPGKSPKLLIFAASTLQGGIPSRFSGSGSGTEFTLTISSLRP
EDFATYYCQHLNTYPYTFGQGTKLEIKR;
SEQ ID NO: 59
CAG GAC GTT AGC AGT TAT;
SEQ ID NO: 60
Q D V S S Y;
SEQ ID NO: 61
GCT GCA TCC;
SEQ ID NO: 62
A A S;
SEQ ID NO: 63
CAA CAC CTT AAT ACT TAC CCG TAC ACT;
SEQ ID NO: 64
Q H L N T Y P Y T;
SEQ ID NO: 65
CAGGTGCAGCTGGTGGAGTCTGGGGGAGGCGTGGTCCAGTCTGGGAGGTCCCTGAGACTCTCCTGTGCAGCGTCTTCATT
CACCTTCAGTAGCTATGGCATGCACTGGGTCCGCCAGTCTCCAGGCAAGGGGCTGGAGTGGGTGGCAGTTATAAGTTATG
ATGGAAGTAATAAATACTATGGAGACTTCGTGAGGGGCCGATTCACCATCTCCAGAGACAATTCCAAGAACACGCTGTAT
CTGCAAATGAACAGCCTGAGAGCCGAGGACACGGCTATGTATTATTGTGCGAGAGAAGTTCGTCGCTACTATTATTACGG
TATGGACGTCTGGGGCCAAGGGACCACGGTCACCGTCTCCTCA;
SEQ ID NO: 66
QVQLVESGGGVVQSGRSLRLSCAASSFTFSSYGMHWVRQSPGKGLEWVAVISYDGSNKYYGDFVRGRFTISRDNSKNTLY
LQMNSLRAEDTAMYYCAREVRRYYYYGMDVWGQGTTVTVSS;
SEQ ID NO: 67
TCA TTC ACC TTC AGT AGC TAT GGC;
SEQ ID NO: 68
S F T F S S Y G;
SEQ ID NO: 69
ATA AGT TAT GAT GGA AGT AAT AAA;
SEQ ID NO: 70
I S Y D G S N K;
SEQ ID NO: 71
GCG AGA GAA GTT CGT CGC TAC TAT TAT TAC GGT ATG GAC GTC;
SEQ ID NO: 72
A R E V R R Y Y Y Y G M D V;
SEQ ID NO: 73
GACATCCAGATGACCCAGTCTCCATCCTCCCTGTCTGCATCTGTAGGAGACAGAGTCACCATCACTTGCCAGGCGAGTCA
GGACATTAGTAATTATTTAAATTGGTATCTGCAGAAACCAGGGAAAGCCCCTAAGCTCCTGATCTCCGATGCATCCAATT
TGGAAACAGGGGTCCCATCAAGGTTCAGTGGAAGTGGATCTGGGACAGATTTTACTTTCACCATCAGCAGCCTGCAGCCT
GAAGATATTGCAACATATTACTGTCAACAGTATAATAATCTCCCGCTCACTTTCGGCGGAGGGACCAAGGTGGAGATCAA
ACGA;
SEQ ID NO: 74
DIQMTQSPSSLSASVGDRVTITCQASQDISNYLNWYLQKPGKAPKLLISDASNLETGVPSRFSGSGSGTDFTFTISSLQP
EDIATYYCQQYNNLPLTFGGGTKVEIKR;
SEQ ID NO: 75
CAG GAC ATT AGT AAT TAT;
SEQ ID NO: 76
Q D I S N Y;
SEQ ID NO: 77
GAT GCA TCC;
SEQ ID NO: 78
D A S;
SEQ ID NO: 79
CAA CAG TAT AAT AAT CTC CCG CTC ACT;
SEQ ID NO: 80
Q Q Y N N L P L T;
SEQ ID NO: 81
GAAGTGCAGCTGGTGGAGTCTGGGGGAGGCTTGGTACAGCCTGGCAGGTCCCTGAGACTCTCCTGTGCAGTCTCTGGATT
CACCTTTGATGATTATGCCATGCACTGGGTCCGGCAAGCTCCAGGGAAGGGCCTGGAGTGGGTCTCAGGTATTAGTTGGA
ATAGTGGTGACATAGACTATGCGGACTCTGTGAAGGGCCGATTCACCATTTCCAGAGACAACGCCAAGAACTCCCTGTAT
CTGCAAATGAACAGTCTGAGAGCTGAGGACACGGCCTTGTATTACTGTGCAAAAGATACCCTCTCAGGGACTGGAACTAC
GTGGTACTATTTTGACTACTGGGGCCAGGGAACCCTGGTCACCGTCTCCTCA;
SEQ ID NO: 82
EVQLVESGGGLVQPGRSLRLSCAVSGFTFDDYAMHWVRQAPGKGLEWVSGISWNSGDIDYADSVKGRFTISRDNAKNSLY
LQMNSLRAEDTALYYCAKDTLSGTGTTWYYFDYWGQGTLVTVSS;
SEQ ID NO: 83
GGA TTC ACC TTT GAT GAT TAT GCC;
SEQ ID NO: 84
G F T F D D Y A;
SEQ ID NO: 85
ATT AGT TGG AAT AGT GGT GAC ATA;
SEQ ID NO: 86
I S W N S G D I;
SEQ ID NO: 87
GCA AAA GAT ACC CTC TCA GGG ACT GGA ACT ACG TGG TAC TAT TTT GAC TAC;
SEQ ID NO: 88
A K D T L S G T G T T W Y Y F D Y;
SEQ ID NO: 89
GACATCCAGTTGACCCAGTCTCCATCCTTCCTGTCTGCATCTGTAGGAGACAGAGTCACCATCACTTGCTGGGCCAGTCA
GGGTATTAGCAGTTATTTAATCTGGTATCAGCAAAAACCAGGGAAAGCCCCTAAGCTCCTGATCTATGCTGCATCCACTT
TGCAAAGTGGGGTCCCATCAAGGTTCAGCGGCAGTGGATCTGGGACAGAATTCACTCTCACAATCAGCAGCCTGCAGCCT
GAAGATTTTGCAACTTATTACTGTCAACAGGTGAATAGTTACCCTCTCACTTTCGGCGGAGGGACCAAGGTGGAGATCAA
ACGA;
SEQ ID NO: 90
DIQLTQSPSFLSASVGDRVTITCWASQGISSYLIWYQQKPGKAPKLLIYAASTLQSGVPSRFSGSGSGTEFTLTISSLQP
EDFATYYCQQVNSYPLTFGGGTKVEIKR;
SEQ ID NO: 91
CAG GGT ATT AGC AGT TAT;
SEQ ID NO: 92
Q G I S S Y;
SEQ ID NO: 93
GCT GCA TCC;
SEQ ID NO: 94
A A S;
SEQ ID NO: 95
CAA CAG GTG AAT AGT TAC CCT CTC ACT;
SEQ ID NO: 96
Q Q V N S Y P L T;
SEQ ID NO: 97
CAGGTGCAGCTGGTGGAGTCTGGGGGAGGCGTGGTCCAGCCTGGGAGGTCCCTGAGACTCTCCTGTGCAGCCTCTGGATT
CACCTTCAGTAGTTATGGCATGCACTGGGTCCGCCAGGCTCCAGGCAAGGGACTGGAGTGGATGGCAGTTATATCATATG
ATGGAAGTAATAAATATTATGCAGACTCCGTGAAGGGCCGATTCACCATCTCCAGAGACAATTCCAAGAACACGCTGTTT
CTGCAAATGAACAGCCTGAGACCTGAAGACACGGCTGTATATTACTGTGCGCAAGATGGCAGCTCGGCGATTTACTATTT
CTACGGTATGGACGTCTGGGGCCAAGGGACCACGGTCACCGTCTCCTCA;
SEQ ID NO: 98
QVQLVESGGGVVQPGRSLRLSCAASGFTFSSYGMHWVRQAPGKGLEWMAVISYDGSNKYYADSVKGRFTISRDNSKNTLF
LQMNSLRPEDTAVYYCAQDGSSAIYYFYGMDVWGQGTTVTVSS;
SEQ ID NO: 99
GGA TTC ACC TTC AGT AGT TAT GGC;
SEQ ID NO: 100
G F T F S S Y G;
SEQ ID NO: 101
ATA TCA TAT GAT GGA AGT AAT AAA;
SEQ ID NO: 102
I S Y D G S N K;
SEQ ID NO: 103
GCG CAA GAT GGC AGC TCG GCG ATT TAC TAT TTC TAC GGT ATG GAC GTC;
SEQ ID NO: 104
A Q D G S S A I Y Y F Y G M D V;
SEQ ID NO: 105
GACATCCAGATGACCCAGTCTCCATCCTCACTGTCTGCATCTATAGGAGACAGAGTCACCATCACTTGTCGGGCGAGTCA
GGACATCAACAATTATTTAGCCTGGTTTCAGCAGAAACCAGGGAAAGCCCCTAAGTCCCTGATCTATGCTGCATCCAGTT
TGCAAAGTGGGGTCCCATCAAAGTTCAGCGGCAGTGGATCTGGGACAGATTTCACTCTCACCATCAGCAGCCTGCAGCCT
GAAGATTTTGCAACTTATTACTGCCTTCAGTATAATAGTTACCATCCCACTTTTGGCCAGGGGACCAAGCTGGAGATCAA
ACGA;
SEQ ID NO: 106
DIQMTQSPSSLSASIGDRVTITCRASQDINNYLAWFQQKPGKAPKSLIYAASSLQSGVPSKFSGSGSGTDFTLTISSLQP
EDFATYYCLQYNSYHPTFGQGTKLEIKR;
SEQ ID NO: 107
CAG GAC ATC AAC AAT TAT;
SEQ ID NO: 108
Q D I N N Y;
SEQ ID NO: 109
GCT GCA TCC;
SEQ ID NO: 110
A A S;
SEQ ID NO: 111
CTT CAG TAT AAT AGT TAC CAT CCC ACT;
SEQ ID NO: 112
L Q Y N S Y H P T;
SEQ ID NO: 113
CAGGTGCAGCTGGTGGAGTCTGGGGGAGGCGTGGTCCAGCCTGGGAGGTCCCTGAGACTCTCCTGTGCAGCGTCTGGATT
CACCTTCAGTAGCTATGGCATGCACTGGGTCCGCCAGGCTCCAGGCAAGGGGCTGGAGTGGGTGGCAGCTATATGGTATG
ATGGAAGTAATAAATATTATGCAGACTCCGTGAAGGGCCGATTCACCATCTCCAGAGACAATTCCAAGAACACGCTGTAT
CTGCAAATGAACAGCCTGAGAGCCGAGGACACGGCTGTGTATTTCTGTGCGAGAGGGGAACATTACTATGGTTCGGGGCC
GTTCGACCCCTGGGGCCAGGGAACCCTGGTCACCGTCTCCTCA;
SEQ ID NO: 114
QVQLVESGGGVVQPGRSLRLSCAASGFTFSSYGMHWVRQAPGKGLEWVAAIWYDGSNKYYADSVKGRFTISRDNSKNTLY
LQMNSLRAEDTAVYFCARGEHYYGSGPFDPWGQGTLVTVSS;
SEQ ID NO: 115
GGA TTC ACC TTC AGT AGC TAT GGC;
SEQ ID NO: 116
G F T F S S Y G;
SEQ ID NO: 117
ATA TGG TAT GAT GGA AGT AAT AAA;
SEQ ID NO: 118
I W Y D G S N K;
SEQ ID NO: 119
GCG AGA GGG GAA CAT TAC TAT GGT TCG GGG CCG TTC GAC CCC;
SEQ ID NO: 120
A R G E H Y Y G S G P F D P;
SEQ ID NO: 121
GACATCCAGATGACCCAGTCTCCATCCTCCCTGTCTGCATCTGTAGGAGACAGAGTCACCATCACTTGCCGGGCAAGTCA
GAGCATTAGCAACTATTTAAATTGGTATCAGCAGAAACCAGGGAAAGCCCCTAAGCTCCTGATCTTTGCTGCATCCAGTT
TACAAAGTGGGGTCCCATCAAGGTTCAGTGGCAGTGGATCTGGGACAGATTTCACTCTCACCATCAGCAGTCTGCAACCT
GAAGATTTTGCAACTTACTACTGTCAACAGAGTTACAGTTCCCCGCTCACTTTCGGCGGAGGGACCAAGGTGGAGATCAA
ACGA;
SEQ ID NO: 122
DIQMTQSPSSLSASVGDRVTITCRASQSISNYLNWYQQKPGKAPKLLIFAASSLQSGVPSRFSGSGSGTDFTLTISSLQP
EDFATYYCQQSYSSPLTFGGGTKVEIKR;
SEQ ID NO: 123
CAG AGC ATT AGC AAC TAT;
SEQ ID NO: 124
Q S I S N Y;
SEQ ID NO: 125
GCT GCA TCC;
SEQ ID NO: 126
A A S;
SEQ ID NO: 127
CAA CAG AGT TAC AGT TCC CCG CTC ACT;
SEQ ID NO: 128
Q Q S Y S S P L T;
SEQ ID NO: 129
CAGGTGCAGCTGCAGGAGTCGGGGCCAGGACTGGTGAAGCCTTCACAGACCCTGTCCCTCACCTGCACTGTCTCAGGTGG
CTCCATCAGCAGTTTTGGTTACTACTGGAGCTGGATCCGCCAGCACCCAGGGAAGGGCCTGGAGTGGATTGGGTACATCT
ATTACAGTGGGAGCATCGACTACAACCCGTCCCTCAAGAGTCGAATTACCATATCAGTCGACACGTCTAAGAACCAGTTC
TCCCTGAAGCTGAGCTCTGTGACTGCCGCGGACACGGCCGTGTATTACTGTGCGAGAGAAAGGGACTACGGTGACTACTT
TGACTACTGGGGCCAGGGAACCCTGGTCACCGTCTCCTCA;
SEQ ID NO: 130
QVQLQESGPGLVKPSQTLSLTCTVSGGSISSFGYYWSWIRQHPGKGLEWIGYIYYSGSIDYNPSLKSRITISVDTSKNQF
SLKLSSVTAADTAVYYCARERDYGDYFDYWGQGTLVTVSS;
SEQ ID NO: 131
GGT GGC TCC ATC AGC AGT TTT GGT TAC TAC;
SEQ ID NO: 132
G G S I S S F G Y Y;
SEQ ID NO: 133
ATC TAT TAC AGT GGG AGC ATC;
SEQ ID NO: 134
I Y Y S G S I;
SEQ ID NO: 135
GCG AGA GAA AGG GAC TAC GGT GAC TAC TTT GAC TAC;
SEQ ID NO: 136
A R E R D Y G D Y F D Y;
SEQ ID NO: 137
GAAATAGTGATGACGCAGTCTCCAGCCACCCTGTCTGTCTCTCCAGGGGAAAGAGCCACCCTCTCCTGCAGGGCCAGTCA
GAGTGTTAGCAGCAACTTAGCCTGGTACCAGCAGAAACCTGGCCAGGCTCCCAGGCTCCTCATCTATGGTGCATCCACCA
GGGCCACTGGTGTCCCAGCCAGGTTCAGTGGCAGTGGGTCTGGGACAGAGTTCACTCTCACCATCAGCAGTTTGCAGTCT
GAGGATTTTGCAGTTTATTCCTGTCAGCAGTATAATAACTGGCCTCTCACTTTCGGCGGAGGGACCAAGGTGGAGATCAA
ACGA;
SEQ ID NO: 138
EIVMTQSPATLSVSPGERATLSCRASQSVSSNLAWYQQKPGQAPRLLIYGASTRATGVPARFSGSGSGTEFTLTISSLQS
EDFAVYSCQQYNNWPLTFGGGTKVEIKR;
SEQ ID NO: 139
CAG AGT GTT AGC AGC AAC;
SEQ ID NO: 140
Q S V S S N;
SEQ ID NO: 141
GGT GCA TCC;
SEQ ID NO: 142
G A S;
SEQ ID NO: 143
CAG CAG TAT AAT AAC TGG CCT CTC ACT;
SEQ ID NO: 144
Q Q Y N N W P L T;
SEQ ID NO: 145
CAGGTGCAGCTGGTGGAGTCTGGGGGAGGCGTGGTCCAGCCTGGGAGGTCCCTGAGACTCTCCTGTGCAGCGTCTGGATT
CACCTTCAGTAGCTATGGCATACACTGGGTCCGCCAGGCTCCAGGCAAGGGGCTGGAGTGGGTGGCACTTATATGGTATG
ATGGAAGTAATAAATACTATGCAGACTCCGTGAAGGGCCGATTCACCATCTCCAGAGACAATTCCAAGAACACGCTGTAT
CTGCAAATGAACAGCCTGAGAGCCGAGGACACGGCTGTGTATTACTGTGCGAGAGATCAGGATTACTATGGTTCGGGGAG
TTCCTACGGTATGGACGTCTGGGGCCAAGGGACCACGGTCACCGTCTCCTCA;
SEQ ID NO: 146
QVQLVESGGGVVQPGRSLRLSCAASGFTFSSYGIHWVRQAPGKGLEWVALIWYDGSNKYYADSVKGRFTISRDNSKNTLY
LQMNSLRAEDTAVYYCARDQDYYGSGSSYGMDVWGQGTTVTVSS;
SEQ ID NO: 147
GGA TTC ACC TTC AGT AGC TAT GGC;
SEQ ID NO: 148
G F T F S S Y G;
SEQ ID NO: 149
ATA TGG TAT GAT GGA AGT AAT AAA;
SEQ ID NO: 150
I W Y D G S N K;
SEQ ID NO: 151
GCG AGA GAT CAG GAT TAC TAT GGT TCG GGG AGT TCC TAC GGT ATG GAC GTC;
SEQ ID NO: 152
A R D Q D Y Y G S G S S Y G M D V;
SEQ ID NO: 153
GACATCCAGATGACCCAGTCGCCAGCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCGCCTGATCTATGCTGCATCCAGTT
TGCAAAGTGGGGTCCCATCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCTCACTTTTGGCCAGGGGACCAAGCTGGAGATCAA
ACGA;
SEQ ID NO: 154
DIQMTQSPASLSASVGDRVTITCRASQSISSYLNWYQQKPGKAPKRLIYAASSLQSGVPSRFSGSGSGTDFTLTISSLQP
EDFATYYCQQSYSTPLTFGQGTKLEIKR;
SEQ ID NO: 155
CAG AGC ATT AGC AGC TAT;
SEQ ID NO: 156
Q S I S S Y;
SEQ ID NO: 157
GCT GCA TCC;
SEQ ID NO: 158
A A S;
SEQ ID NO: 159
CAA CAG AGT TAC AGT ACC CCT CTC ACT;
SEQ ID NO: 160
Q Q S Y S T P L T;
SEQ ID NO: 161
CAGGTGCAGCTGGTGGAGTCTGGGGGAGGCGTGGTCCAGCCTGGGAGGTCCCTGAGACTCTCCTGTGCAGCGTCTGGATT
CACCTTCAGTACCTATGGCATGCACTGGGTCCGCCAGGCTCCAGGCAAGGGGCTGGAGTGGGTGGCAGTTATATGGTATG
ATGGAACTAATAAATACTATGCAGACTCCGTGAAGGGCCGATTCACCATCTCCAGAGACAATTCCAAGAACACGCTGTAT
CTGCAAATGATCAGCCTGAGAGCCGAGGACACGGCTGTGTATTACTGTGCGAGAGACCCCTCAGGTGGGGACCACTACTA
TTACTACGGTATGGACGTCTGGGGCCAAGGGACCACGGTCACCGTCTCCTCA;
SEQ ID NO: 162
QVQLVESGGGVVQPGRSLRLSCAASGFTFSTYGMHWVRQAPGKGLEWVAVIWYDGTNKYYADSVKGRFTISRDNSKNTLY
LQMISLRAEDTAVYYCARDPSGGDHYYYYGMDVWGQGTTVTVSS;
SEQ ID NO: 163
GGA TTC ACC TTC AGT ACC TAT GGC;
SEQ ID NO: 164
G F T F S T Y G;
SEQ ID NO: 165
ATA TGG TAT GAT GGA ACT AAT AAA;
SEQ ID NO: 166
I W Y D G T N K;
SEQ ID NO: 167
GCG AGA GAC CCC TCA GGT GGG GAC CAC TAC TAT TAC TAC GGT ATG GAC GTC;
SEQ ID NO: 168
A R D P S G G D H Y Y Y Y G M D V;
SEQ ID NO: 169
GACATCCAGATGACCCAGTCTCCATCCTCCCTGTCTGCATCTGTAGGAGACAGAATCACCATCACTTGCCAGGCGAGTCA
GGACATTAGCAACTATTTAAATTGGTATCAGCAGAAGCCAGGGAAAGCCCCTAACCTCCTGATCTCCGATGCATCCGATT
TGGAAACAGGGGTCCCATCAAGGTTCAGTGGAAGTGGATCTGGGACAGATTTTACTTTCACCATCAGCAGCCTGCAGCCT
GAAGATTTTGCAACATATTACTGTCAACAGTATGATAATATACCGATCACCTTCGGCCAAGGGACACGACTGGAGATTAA
ACGA;
SEQ ID NO: 170
DIQMTQSPSSLSASVGDRITITCQASQDISNYLNWYQQKPGKAPNLLISDASDLETGVPSRFSGSGSGTDFTFTISSLQP
EDFATYYCQQYDNIPITFGQGTRLEIKR;
SEQ ID NO: 171
CAG GAC ATT AGC AAC TAT;
SEQ ID NO: 172
Q D I S N Y;
SEQ ID NO: 173
GAT GCA TCC;
SEQ ID NO: 174
D A S;
SEQ ID NO: 175
CAA CAG TAT GAT AAT ATA CCG ATC ACC;
SEQ ID NO: 176
Q Q Y D N I P I T;
SEQ ID NO: 177
CAGGTGCAGCTGGTGGAGTCAGGGGGAGGCGTGGTCCAGCCTGGGAGGTCCCTGAGACTCTCCTGTGCAGCCTCTGGATT
CACCTTCAGTAGCTATGGCATGCACTGGGTCCGCCAGGCTCCAGGCAAGGGGCTGGAGTGGGTGACATTTATATCATTTG
ATGAAAGGAATAAATACTATGCAGACTCCGTTAAGGGCCGATTCACCATCTCCAGAGACAATTCCAAGAACACGCTGTAT
CTGCAAATGAACAGCCTGAGAGCTGAGGACACGGCTATGTATTACTGTGCGAGCGAAGTCGGGTACAGTTTTGGTCATGA
TGCTTTTGATATCTGGGGCCAAGGGACAATGGTCACCGTCTCTTCA;
SEQ ID NO: 178
QVQLVESGGGVVQPGRSLRLSCAASGFTFSSYGMHWVRQAPGKGLEWVTFISFDERNKYYADSVKGRFTISRDNSKNTLY
LQMNSLRAEDTAMYYCASEVGYSFGHDAFDIWGQGTMVTVSS;
SEQ ID NO: 179
GGA TTC ACC TTC AGT AGC TAT GGC;
SEQ ID NO: 180
G F T F S S Y G;
SEQ ID NO: 181
ATA TCA TTT GAT GAA AGG AAT AAA;
SEQ ID NO: 182
I S F D E R N K;
SEQ ID NO: 183
GCG AGC GAA GTC GGG TAC AGT TTT GGT CAT GAT GCT TTT GAT ATC;
SEQ ID NO: 184
A S E V G Y S F G H D A F D I;
SEQ ID NO: 185
GACATCCAGATGACCCAGTCTCCATCCTCCCTGTCTGCATCTGTAGGAGACAGAGTCACCATCACTTGCCAGGCGAGTCA
GGACATTAGCAACTATTTAAATTGGTATCAGAAGAAACCAGGGAAAGCCCCTAAACTCCTGATCTACGATGCATCCAATT
TGGAAACAGGGGTCCCGTCAAGGTTCAGTGGAAGTGGATCTGGGACAGATTTTACTTTCACCATCAGCAGCCTGCAGCCT
GAAGATATTGCAACATATTACTGTCAACAGTATGATAATTTCCCGCTCACTTTCGGCGGAGGGACCAAGGTGGAGATCAA
ACGA;
SEQ ID NO: 186
DIQMTQSPSSLSASVGDRVTITCQASQDISNYLNWYQKKPGKAPKLLIYDASNLETGVPSRFSGSGSGTDFTFTISSLQP
EDIATYYCQQYDNFPLTFGGGTKVEIKR;
SEQ ID NO: 187
CAG GAC ATT AGC AAC TAT;
SEQ ID NO: 188
Q D I S N Y;
SEQ ID NO: 189
GAT GCA TCC;
SEQ ID NO: 190
D A S;
SEQ ID NO: 191
CAA CAG TAT GAT AAT TTC CCG CTC ACT;
SEQ ID NO: 192
Q Q Y D N F P L T;
SEQ ID NO: 193
GAGGTGCAGCTGGTGGAGTCTGGGGGAGGCTTGGTACAGCCTGGGGGGTCCCTGAGACTCTCCTGTGCAGCCTCTGGATT
CACCTTTAACAACTATGCCATGAGCTGGGTCCGCCAGGCTCCAGGGAGGGGGCTGGAGTGGGTCTCAGCTATTAGTGGTA
GTGGTGATAGCACATACTACTCAGACTCCGTGAAGGGCCGGTTCACCATCTCCAGAGACAATTCCAAGAACACGCTGTAT
CTGCAAATGAACAGCCTGAGAGCCGAGGACACGGCCGTATATTACTGTGCGAAAGATCAGGGCCTGTATTACTATGGTTC
GGGGAGTTTTGACTACTGGGGCCAGGGAACCCTGGTCACCGTCTCCTCA;
SEQ ID NO: 194
EVQLVESGGGLVQPGGSLRLSCAASGFTFNNYAMSWVRQAPGRGLEWVSAISGSGDSTYYSDSVKGRFTISRDNSKNTLY
LQMNSLRAEDTAVYYCAKDQGLYYYGSGSFDYWGQGTLVTVSS;
SEQ ID NO: 195
GGA TTC ACC TTT AAC AAC TAT GCC;
SEQ ID NO: 196
G F T F N N Y A;
SEQ ID NO: 197
ATT AGT GGT AGT GGT GAT AGC ACA;
SEQ ID NO: 198
I S G S G D S T;
SEQ ID NO: 199
GCG AAA GAT CAG GGC CTG TAT TAC TAT GGT TCG GGG AGT TTT GAC TAC;
SEQ ID NO: 200
A K D Q G L Y Y Y G S G S F D Y;
SEQ ID NO: 201
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCCAAGCTGCATCCAGTT
TGCAAAGTGGGGTCCCATCAAGGTTCAGTGGCAGTGGATCTGGGACAGATATCACTCTCACCATCAGCAGTCTGCAACCC
GAAGATTTTGCAACTTACTACTGTCAACAGAGTTACAGTACCCCATTCACTTTCGGCCCTGGGACCAAAGTGGATATCAA
ACGA;
SEQ ID NO: 202
DIQMTQSPSSLSASVGDRVTITCRASQSISSYLNWYQQKPGKAPKLLIQAASSLQSGVPSRFSGSGSGTDITLTISSLQP
EDFATYYCQQSYSTPFTFGPGTKVDIKR;
SEQ ID NO: 203
CAG AGC ATT AGC AGC TAT;
SEQ ID NO: 204
Q S I S S Y;
SEQ ID NO: 205
GCT GCA TCC;
SEQ ID NO: 206
A A S;
SEQ ID NO: 207
CAA CAG AGT TAC AGT ACC CCA TTC ACT;
SEQ ID NO: 208
Q Q S Y S T P F T;
SEQ ID NO: 209
GAGGTGCAGCTGGTGGAGTCTGGGGGAGGCTTGGTCCAGCCTGGGGGGTCCCTGCAACTCTCCTGTGCAGCCTCTGGGTT
TGCCTTCAGCGACTCTGCTATATACTGGGTCCGCCAGGCTTCCGGGAAAGGGCTGGAGTGGGTTGGCCGCATTAGAAACA
AAGCTAATAGGTTCGCGACAGCATATGGTGCGTCGGTGAAAGGCAGGTTCAGCATACACAGAGATGATTCAAAGAACACG
GCGTATCTACAAATGAACAGCCTGAAAACCGAGGACACGGCCGTGTATTACTGTGCCAGACATGGACACGATACTTTGAC
TGAGGGCTACGGTATGGACGTCTGGGGCCAAGGGACCACGGTCACCGTCTCCTCA;
SEQ ID NO: 210
EVQLVESGGGLVQPGGSLQLSCAASGFAFSDSAIYWVRQASGKGLEWVGRIRNKANRFATAYGASVKGRFSIHRDDSKNT
AYLQMNSLKTEDTAVYYCARHGHDTLTEGYGMDVWGQGTTVTVSS;
SEQ ID NO: 211
GGG TTT GCC TTC AGC GAC TCT GCT;
SEQ ID NO: 212
G F A F S D S A;
SEQ ID NO: 213
ATT AGA AAC AAA GCT AAT AGG TTC GCG ACA;
SEQ ID NO: 214
I R N K A N R F A T;
SEQ ID NO: 215
GCC AGA CAT GGA CAC GAT ACT TTG ACT GAG GGC TAC GGT ATG GAC GTC;
SEQ ID NO: 216
A R H G H D T L T E G Y G M D V;
Light chain #1-
SEQ ID NO: 217
GACATCCAGATGACCCAGTCTCCATCCTCCCTGTCTGCATCTGTAGGAGACAGAGTCACCATCACTTGCCGGGCAAGTCA
GAGCATTAGCAGCTATTTAAATTGGTATCAGCAGAAACCAGGGAAAGCCCCTAAGCTCCTGATCTATGCTGCATCCAGTT
TGCAAAGTGGGGTCCCGTCAAGGTTCAGTGGCAGTGGATCTGGGACAGATTTCACTCTCACCATCAGCAGTCTGCAACCT
GAAGATTTTGCAACTTACTACTGTCAACAGAGTTACAGTACCCCTCCGATCACCTTCGGCCAAGGGACACGACTGGAGAT
TAAA;
Light chain #1-
SEQ ID NO: 218
DIQMTQSPSSLSASVGDRVTITCRASQSISSYLNWYQQKPGKAPKLLIYAASSLQSGVPSRFSGSGSGTDFTLTISSLQP
EDFATYYCQQSYSTPPITFGQGTRLEIK;
SEQ ID NO: 219
CAG AGC ATT AGC AGC TAT;
SEQ ID NO: 220
Q S I S S Y;
SEQ ID NO: 221
GCT GCA TCC;
SEQ ID NO: 222
A A S;
SEQ ID NO: 223
CAA CAG AGT TAC AGT ACC CCT CCG ATC ACC;
SEQ ID NO: 224
Q Q S Y S T P P I T;
SEQ ID NO: 225
CAGGTGCAGCTGGTGCAGTCTGGGGCTGAGGTAAAGAACCCTGGGTCCTCGGTGAAGGTCTCCTGCAAGGCTTCTGGAGG
CACCTTCAGCAGTTATACTATCAACTGGGTGCGACAGGCCCCTGGACAAGGGCTTGAGTGGATGGGAGGGATCATCCCTC
TCTATGGAACAGCAAACTACGCACAGAAGTTCCAGGCCAGAGTCACGATTTCCACGGACGAATCCACGAACACAGCCTAC
ATGGAACTGAGCAACCTGAGATTTGAAGACACGGCCGTGTATTTCTGTGCGAGTACACTGGAACTACGGGCTTTTGATGC
CTTTGATATCTGGGGCCAAGGGACAATGGTCACCGTCTCTTCA;
SEQ ID NO: 226
QVQLVQSGAEVKNPGSSVKVSCKASGGTFSSYTINWVRQAPGQGLEWMGGIIPLYGTANYAQKFQARVTISTDESTNTAY
MELSNLRFEDTAVYFCASTLELRAFDAFDIWGQGTMVTVSS;
SEQ ID NO: 227
GGA GGC ACC TTC AGC AGT TAT ACT;
SEQ ID NO: 228
G G T F S S Y T;
SEQ ID NO: 229
ATC ATC CCT CTC TAT GGA ACA GCA;
SEQ ID NO: 230
I I P L Y G T A;
SEQ ID NO: 231
GCG AGT ACA CTG GAA CTA CGG GCT TTT GAT GCC TTT GAT ATC;
SEQ ID NO: 232
A S T L E L R A F D A F D I;
SEQ ID NO: 233
CAGGTGCAGCTGCAGGAGTCGGGCCCAGGACTGGTGAAGCCTTCACAGACCCTGTCCCTCACCTGCACTGTCTCTGGTGG
CTCCATCAGCAGTGGTGGTTACTACTGGAACTGGATCCGCCAGCACCCAGGGAAGGGCCTGGAGTGGATTGGGTACATCT
ATTACAGTGGAAGCACCTACTACAACCCGTCCCTCAAGAGTCGAGTTACCATATCAGTAGACACGTCTAAGAACCAGTTC
TCCCTGAAGCTGGGCTCTGTGACTGCCGCGGACACGGCCGTGTATTACTGTGCGCGAGCTCCTCCTTATAACTGGTTTGA
CTACTGGGGCCAGGGAACCCTGGTCACCGTCTCCTCA;
SEQ ID NO: 234
QVQLQESGPGLVKPSQTLSLTCTVSGGSISSGGYYWNWIRQHPGKGLEWIGYIYYSGSTYYNPSLKSRVTISVDTSKNQF
SLKLGSVTAADTAVYYCARAPPYNWFDYWGQGTLVTVSS;
SEQ ID NO: 235
GGT GGC TCC ATC AGC AGT GGT GGT TAC TAC;
SEQ ID NO: 236
G G S I S S G G Y Y;
SEQ ID NO: 237
ATC TAT TAC AGT GGA AGC ACC;
SEQ ID NO: 238
I Y Y S G S T;
SEQ ID NO: 239
GCG CGA GCT CCT CCT TAT AAC TGG TTT GAC TAC;
SEQ ID NO: 240
A R A P P Y N W F D Y;
SEQ ID NO: 241
CAGGTGCAGCTGGTGGAGTCTGGGGGAGGCTTGGTCAAGCCTGGAGGGTCCCTGAGACTCTCCTGTGCAGCCTCTGGATT
CACCTTCAGTGACTACTACATGAACTGGGTCCGCCAGGCTCCAGGGAAGGGGCTGGAATGGGTTTCATACATTAGTAATA
GTGGTAATACCCAATACTACGCAGACTCTGTGAAGGGCCGGTTCACCATCTCCAGGGACAATGCCAAGAACTCCCTGTTT
CTGCAAATGAACAGCCTGCGAGCCGAGGACACGGCCGTTTATTACTGTACGAGAGAGGGACTCGAATATAGCAGCTCGGA
GCCCTTTGACTACTGGGGCCAGGGAACCCTGGTCACCGTCTCCTCA;
SEQ ID NO: 242
QVQLVESGGGLVKPGGSLRLSCAASGFTFSDYYMNWVRQAPGKGLEWVSYISNSGNTQYYADSVKGRFTISRDNAKNSLF
LQMNSLRAEDTAVYYCTREGLEYSSSEPFDYWGQGTLVTVSS;
SEQ ID NO: 243
GGA TTC ACC TTC AGT GAC TAC TAC;
SEQ ID NO: 244
G F T F S D Y Y;
SEQ ID NO: 245
ATT AGT AAT AGT GGT AAT ACC CAA;
SEQ ID NO: 246
I S N S G N T Q;
SEQ ID NO: 247
ACG AGA GAG GGA CTC GAA TAT AGC AGC TCG GAG CCC TTT GAC TAC;
SEQ ID NO: 248
T R E G L E Y S S S E P F D Y;
SEQ ID NO: 249
CAGGTGCAGCTGGTGCAGTCTGGGGCTGAAGTGAAGAAGCCTGGGGCCTCAGTGAAGGTCTCCTGCAAGACTTCTGGATA
CACCTTCACCGCCTACTACATACACTGGGTGCGACAGGCCCCTGGACAAGGGCTTGAGTGGATGGGATGGATCAACCCTA
ACAATGGTGACACAAACTATGCACTGAGGTTTCAGGGCAGGGTCACCATGACCAGGGACATGTCCATCAACACAGCCTAC
ATGGAGCTGCGCGGGCTGAGATCTGACGACACGGCCGTGTATTATTGTGCGAGAGATGATCTAGCAGCAGCGGGTATCGG
CTGGTTCGACTCCTGGGGCCAGGGAACCCTGGTCACCGTCTCCTCA;
SEQ ID NO: 250
QVQLVQSGAEVKKPGASVKVSCKTSGYTFTAYYIHWVRQAPGQGLEWMGWINPNNGDTNYALRFQGRVTMTRDMSINTAY
MELRGLRSDDTAVYYCARDDLAAAGIGWFDSWGQGTLVTVSS;
SEQ ID NO: 251
GGA TAC ACC TTC ACC GCC TAC TAC;
SEQ ID NO: 252
G Y T F T A Y Y;
SEQ ID NO: 253
ATC AAC CCT AAC AAT GGT GAC ACA;
SEQ ID NO: 254
I N P N N G D T;
SEQ ID NO: 255
GCG AGA GAT GAT CTA GCA GCA GCG GGT ATC GGC TGG TTC GAC TCC;
SEQ ID NO: 256
A R D D L A A A G I G W F D S;
SEQ ID NO: 257
GAAGTGCAGCTGGTGGAGTCTGGGGGAGGCTTGCTACAGCCTGGCAGGTCCCTGAGACTCTCCTGTGTAGCCTCTGGATT
CACCTTTGATGATTATGCCATGCACTGGGTCCGGCAAGCTCCAGGGAAGGGCCTGGAGTGGGTCTCAGGAATTAGTTGGA
ATAGTGAAAGTATAGGCTATGCGGACTCTGTGAGGGGCCGATTCACCATTTCCAGAGACAACGCCAAGAACTCCCTGTAT
CTCCAAATGAACAGTCTGAGAGCTGAGGACACGGCCTTGTATTACTGTGCAAAAGCCCCGTATAGTGGGACCTACTTCGA
ATACTTCCGCCACTGGGGCCAGGGCACCCTGGTCACCGTCTCCTCA;
SEQ ID NO: 258
EVQLVESGGGLLQPGRSLRLSCVASGFTFDDYAMHWVRQAPGKGLEWVSGISWNSESIGYADSVRGRFTISRDNAKNSLY
LQMNSLRAEDTALYYCAKAPYSGTYFEYFRHWGQGTLVTVSS;
SEQ ID NO: 259
GGA TTC ACC TTT GAT GAT TAT GCC;
SEQ ID NO: 260
G F T F D D Y A;
SEQ ID NO: 261
ATT AGT TGG AAT AGT GAA AGT ATA;
SEQ ID NO: 262
I S W N S E S I;
SEQ ID NO: 263
GCA AAA GCC CCG TAT AGT GGG ACC TAC TTC GAA TAC TTC CGC CAC;
SEQ ID NO: 264
A K A P Y S G T Y F E Y F R H;
SEQ ID NO: 265
CAGGTGCAGCTGGTGGAGTCTGGGGGAGGCGTGGTCCAGCCTGGGAGGTCCCTGAGACTCTCCTGTGCAGCCTCTGGATT
CACCTTCAGTAGCTATGGCATGCACTGGGTCCGCCAGGCTCCAGGCAAGGGGCTGGAGTGGGTGGCAGTTATATCATATG
ATGGAAGTAATAAATACTATGCAGACTCCGTGAAGGGCCGATTCACCATCTCCAGAGACAATTCCAAGAACACGCTGTAT
CTGCAAATGAACAGCCTGAGAGCTGAGGACACGGCTGTGTATTACTGTGCGAAAGATGACTGGAACTACGACGCCTTTGA
TATCTGGGGCCAAGGGACAATGGTCACCGTCTCTTCA;
SEQ ID NO: 266
QVQLVESGGGVVQPGRSLRLSCAASGFTFSSYGMHWVRQAPGKGLEWVAVISYDGSNKYYADSVKGRFTISRDNSKNTLY
LQMNSLRAEDTAVYYCAKDDWNYDAFDIWGQGTMVTVSS;
SEQ ID NO: 267
GGA TTC ACC TTC AGT AGC TAT GGC;
SEQ ID NO: 268
G F T F S S Y G;
SEQ ID NO: 269
ATA TCA TAT GAT GGA AGT AAT AAA;
SEQ ID NO: 270
I S Y D G S N K;
SEQ ID NO: 271
GCG AAA GAT GAC TGG AAC TAC GAC GCC TTT GAT ATC;
SEQ ID NO: 272
A K D D W N Y D A F D I;
SEQ ID NO: 273
CAGGTGCAGCTGCAGGAGTCGGGCCCAGGACTGGTGAAGCCTTCACAGACCCTGTCCCTCACCTGCACTGTCTCTGGTGG
CTCCATCAGCAGTAGTGGTTACTACTGGAGCTGGATCCGCCAGCACCCAGGGAGGGGCCTGGAGTGGATTGGATACATCT
ATTACAGTGGGAGCACCTACTACAACCCGTCCCTCAAGAGTCGAGTTACCATATCAGTAGACACGTCTAAGAACCAGTTC
TCCCTGAAGCTGAACTCTGTGACTGCCGCGGACACGGCCGTGTATTACTGTGCGAGAGTGGACTATGGTTCGGGGAGTTC
GTTTGACTACTGGGGCCAGGGAACCCTGGTCACCGTCTCCTCA;
SEQ ID NO: 274
QVQLQESGPGLVKPSQTLSLTCTVSGGSISSSGYYWSWIRQHPGRGLEWIGYIYYSGSTYYNPSLKSRVTISVDTSKNQF
SLKLNSVTAADTAVYYCARVDYGSGSSFDYWGQGTLVTVSS;
SEQ ID NO: 275
GGT GGC TCC ATC AGC AGT AGT GGT TAC TAC;
SEQ ID NO: 276
G G S I S S S G Y Y;
SEQ ID NO: 277
ATC TAT TAC AGT GGG AGC ACC;
SEQ ID NO: 278
I Y Y S G S T;
SEQ ID NO: 279
GCG AGA GTG GAC TAT GGT TCG GGG AGT TCG TTT GAC TAC;
SEQ ID NO: 280
A R V D Y G S G S S F D Y;
SEQ ID NO: 281
CAGGTTCAGCTGGTGCAGTCTGGACCTGAGGTGAAGAAGCCTGGGGCCTCAGTGAAGGTCTCCTGCAAGGCTTCTGGTTA
CACCTTTACCAGCTATGGCATCAGCTGGGTGCGACAGGCCCCTGGACAAGGGCTTGAGTGGCTGGGATGGATCAGCGGTT
TCAATGGTAGAACAGACTATACAGAGAAGCTCCAGGACAGAATCACCATGACCACAGACAGATCCTCGAGCACAGCCTAC
ATGGAACTGAGGAGCCTGAGATATGACGACACGGCCGTGTATTACTGTGCGAGAGATGGACTGGAAAAACTTGGTGACTA
CTGGGGCCAGGGAACCCTGGTCACCGTCTCCTCA;
SEQ ID NO: 282
QVQLVQSGPEVKKPGASVKVSCKASGYTFTSYGISWVRQAPGQGLEWLGWISGFNGRTDYTEKLQDRITMTTDRSSSTAY
MELRSLRYDDTAVYYCARDGLEKLGDYWGQGTLVTVSS;
SEQ ID NO: 283
GGT TAC ACC TTT ACC AGC TAT GGC;
SEQ ID NO: 284
G Y T F T S Y G;
SEQ ID NO: 285
ATC AGC GGT TTC AAT GGT AGA ACA;
SEQ ID NO: 286
I S G F N G R T;
SEQ ID NO: 287
GCG AGA GAT GGA CTG GAA AAA CTT GGT GAC TAC;
SEQ ID NO: 288
A R D G L E K L G D Y;
SEQ ID NO: 289
CAGGTGCAGCTGGTGGAGTCTGGGGGAGGCGTGGTCCAGCCGGGGAGGTTCCTGAGACTCTCCTGTGCAGCGTCTGGATT
CACCTTCAGTAACTCTGGCATGCACTGGGTCCGCCAGGCTCCAGGCAAGGGGCTGGAGTGGGTGGCAGGAATATGGCATG
ATGGAAGTTATAAATATTATGTAGACTCCGTGAAGGGCCGATTCACCATCTCCAGAGACAATTCTAAGAACACGCTGTTT
CTGCAAATGAACAGCCTGCGAGCCGAGGACACGGCTGTATATTATTGTGCGAGAGATGATTACTATGCTTCGGGGACCAG
CGTGGACGTATGGGGCCAAGGGACCACGGTCACCGTCTCCTCA;
SEQ ID NO: 290
QVQLVESGGGVVQPGRFLRLSCAASGFTFSNSGMHWVRQAPGKGLEWVAGIWHDGSYKYYVDSVKGRFTISRDNSKNTLF
LQMNSLRAEDTAVYYCARDDYYASGTSVDVWGQGTTVTVSS;
SEQ ID NO: 291
GGA TTC ACC TTC AGT AAC TCT GGC;
SEQ ID NO: 292
G F T F S N S G;
SEQ ID NO: 293
ATA TGG CAT GAT GGA AGT TAT AAA;
SEQ ID NO: 294
I W H D G S Y K;
SEQ ID NO: 295
GCG AGA GAT GAT TAC TAT GCT TCG GGG ACC AGC GTG GAC GTA;
SEQ ID NO: 296
A R D D Y Y A S G T S V D V;
SEQ ID NO: 297
CAGGTGCAGCTGGTGCAGTCTGGGGCTGAGGTGAAGAAGCCTGGGGCCTCAGTGAGGGTCTCCTGCATGGCCTCTGGATA
CACCTTCACCGGCTACTATATGCACTGGGTGCGACAGGCCCCTGGACAAGGGCTTGAGTGGATGGGATGGATCAACCCTA
ACAGTGGTGGCACAAAATATGCACAGAAGTTTCAGGGCAGGGTCACCATGACCAGGGACACGTCCATCAGCACAGCCTAC
ATGGAGCTGAGCAGACTGAGATCTGACGACACGGCCGTATATTACTGTGCGAGAGAAGAAGTCGACGATTTTTGGAGTGG
TTACCTTGACTACTGGGGCCAGGGAACCCTGGTCACCGTCTCCTCA;
SEQ ID NO: 298
QVQLVQSGAEVKKPGASVRVSCMASGYTFTGYYMHWVRQAPGQGLEWMGWINPNSGGTKYAQKFQGRVTMTRDTSISTAY
MELSRLRSDDTAVYYCAREEVDDFWSGYLDYWGQGTLVTVSS;
SEQ ID NO: 299
GGA TAC ACC TTC ACC GGC TAC TAT;
SEQ ID NO: 300
G Y T F T G Y Y;
SEQ ID NO: 301
ATC AAC CCT AAC AGT GGT GGC ACA;
SEQ ID NO: 302
I N P N S G G T;
SEQ ID NO: 303
GCG AGA GAA GAA GTC GAC GAT TTT TGG AGT GGT TAC CTT GAC TAC;
SEQ ID NO: 304
A R E E V D D F W S G Y L D Y;
Light chain #2-
SEQ ID NO: 305
GAAATTGTGTTGACGCAGTCTCCAGGCACCCTGTCTTTGTCTCCAGGGGAAAGAGCCACCCTCTCCTGCAGGGCCAGTCA
GAGTGTTAGCAGCAGCTACTTAGCCTGGTACCAGCAGAAACCTGGCCAGGCTCCCAGGCTCCTCATCTATGGTGCATCCA
GCAGGGCCACTGGCATCCCAGACAGGTTCAGTGGCAGTGGGTCTGGGACAGACTTCACTCTCACCATCAGCAGACTGGAG
CCTGAAGATTTTGCAGTGTATTACTGTCAGCAGTATGGTAGCTCACCTTGGACGTTCGGCCAAGGGACCAAGGTGGAAAT
CAAA;
Light chain #2-
SEQ ID NO: 306
EIVLTQSPGTLSLSPGERATLSCRASQSVSSSYLAWYQQKPGQAPRLLIYGASSRATGIPDRFSGSGSGTDFTLTISRLE
PEDFAVYYCQQYGSSPWTFGQGTKVEIK;
SEQ ID NO: 307
CAG AGT GTT AGC AGC AGC TAC;
SEQ ID NO: 308
Q S V S S S Y;
SEQ ID NO: 309
GGT GCA TCC;
SEQ ID NO: 310
G A S;
SEQ ID NO: 311
CAG CAG TAT GGT AGC TCA CCT TGG ACG;
SEQ ID NO: 312
Q Q Y G S S P W T;
SEQ ID NO: 313
GAGGTGCAGCTGGTGGAGTCTGGAGGAGACTTGGTCCAGCCGGGGGGGTCCCTGAGACTCTCCTGTGCAGCCTCTGGGTT
CGCCGTCAATGGCGACTATTTTAGTTGGGTCCGCCAGGCTCCAGGGAAGGGGCTGGAGTGGATCTCAGTTATTTATAGCA
GTGGTAACACATACTACGCAGACTCCGTGAAGGGCCGATTCACCATCTCCAGACACAATTCCAAGAACACGCTGTATCTT
CAAATGAGCAGCCTAAGACCTGAGGACACGGCCGTGTATTACTGTGCGAGAGACTTCCCTCCAATGTCTGGTGCGGACTA
CTGGGGCCAGGGAACCCTGGTCACCGTCTCCTCA;
SEQ ID NO: 314
EVQLVESGGDLVQPGGSLRLSCAASGFAVNGDYFSWVRQAPGKGLEWISVIYSSGNTYYADSVKGRFTISRHNSKNTLYL
QMSSLRPEDTAVYYCARDFPPMSGADYWGQGTLVTVSS;
SEQ ID NO: 315
GGG TTC GCC GTC AAT GGC GAC TAT;
SEQ ID NO: 316
G F A V N G D Y;
SEQ ID NO: 317
ATT TAT AGC AGT GGT AAC ACA;
SEQ ID NO: 318
I Y S S G N T;
SEQ ID NO: 319
GCG AGA GAC TTC CCT CCA ATG TCT GGT GCG GAC TAC;
SEQ ID NO: 320
A R D F P P M S G A D Y;
SEQ ID NO: 321
CAGGTCCAGCTGGTGCAGTCTGGGGCTGAGGTGAAGAAGCCTGGGGCCTCAGTGAAGGTCTCCTGCAAGGTTTCCGGATA
CACCCTCACTGAATTGTCCATGCACTGGGTGCGACAGGCTCCTGGAAAAGGGCTTGAATGGATGGGAGGTTTTGATCCTG
AACATGGTAAAATAATCTACGCACAGAAATTCCAGGGCAGAGTCACCATGACCGAGGACACATCTACAGACACAGCCTAC
ATGGAACTGAGCAGCCTGAGATCTGAGGACACGGCCGTCTATTACTGTGCAACATTTTATAACTGGAACTCCTACTACTT
CGGTATGGACGTCTGGGGCCACGGGACCACGGTCACCGTCTCCTCA;
SEQ ID NO: 322
QVQLVQSGAEVKKPGASVKVSCKVSGYTLTELSMHWVRQAPGKGLEWMGGFDPEHGKIIYAQKFQGRVTMTEDTSTDTAY
MELSSLRSEDTAVYYCATFYNWNSYYFGMDVWGHGTTVTVSS;
SEQ ID NO: 323
GGA TAC ACC CTC ACT GAA TTG TCC;
SEQ ID NO: 324
G Y T L T E L S;
SEQ ID NO: 325
TTT GAT CCT GAA CAT GGT AAA ATA;
SEQ ID NO: 326
F D P E H G K I;
SEQ ID NO: 327
GCA ACA TTT TAT AAC TGG AAC TCC TAC TAC TTC GGT ATG GAC GTC;
SEQ ID NO: 328
A T F Y N W N S Y Y F G M D V;
SEQ ID NO: 329
GAGGTGCAGCTGGTGGAGTCGGGGGGAGGCTTGGTACAGCCTGGGGGGTCCCTGAGACTCTCCTGTGCAGCCTCTGGATT
CACCTTTAGCAGCTATGCCATGAACTGGGTCCGCCAGGCTCCAGGAAAGGGACTGGAGTGGGTCTCAGCTGTTAGTGGAA
GTGCTGATATCACAAACTACGCAGACTCCGTGAAGGGCCGGTTCACCATCTCCAGAGACAATTCCAAACACACGCTGTAT
CTGCAAATGAACAGCCTGAGAGCCGAGGACACGGCCGTATATTACTGTGCGAAGGATAAAGTGTATAACTGGAACTACGG
GATCTACTACGGTATGGACGTCTGGGGCCAAGGGACCACGGTCACCGTCTCCTCA;
SEQ ID NO: 330
EVQLVESGGGLVQPGGSLRLSCAASGFTFSSYAMNWVRQAPGKGLEWVSAVSGSADITNYADSVKGRFTISRDNSKHTLY
LQMNSLRAEDTAVYYCAKDKVYNWNYGIYYGMDVWGQGTTVTVSS;
SEQ ID NO: 331
GGA TTC ACC TTT AGC AGC TAT GCC;
SEQ ID NO: 332
G F T F S S Y A;
SEQ ID NO: 333
GTT AGT GGA AGT GCT GAT ATC ACA;
SEQ ID NO: 334
V S G S A D I T;
SEQ ID NO: 335
GCG AAG GAT AAA GTG TAT AAC TGG AAC TAC GGG ATC TAC TAC GGT ATG GAC GTC;
SEQ ID NO: 336
A K D K V Y N W N Y G I Y Y G M D V;
SEQ ID NO: 337
CAGCTGCAGCTGCAGGAGTCGGGCCCAGGACTGGTGAAGCCTTCGGAGACCCTGTCCCTCACCTGCACTGTCTCTGGTGG
CTCCATCAGCAGTAGTAGTTACTACTGGGGCTGGATCCGCCAGCCCCCAGGGAAGGGACTAGAGTGGATTGGGAGTATCT
ATTATAGTGGGAGCACCTACTACAATCCGTCCCTCAAGAGTCGAGTCACCATATCCGTAGACACGTCCAAGAACCAGTTC
TCCCTGAAGCTGAGCTCTGTGACCGCCGCAGACACGGCTGTGTATTACTGTGCGAGACAAGGGAGGTGGGAGCGAGAAAA
CTTTGACTACTGGGGCCAGGGAACCCTGGTCACCGTCTCCTCA;
SEQ ID NO: 338
QLQLQESGPGLVKPSETLSLTCTVSGGSISSSSYYWGWIRQPPGKGLEWIGSIYYSGSTYYNPSLKSRVTISVDTSKNQF
SLKLSSVTAADTAVYYCARQGRWERENFDYWGQGTLVTVSS;
SEQ ID NO: 339
GGT GGC TCC ATC AGC AGT AGT AGT TAC TAC;
SEQ ID NO: 340
G G S I S S S S Y Y;
SEQ ID NO: 341
ATC TAT TAT AGT GGG AGC ACC;
SEQ ID NO: 342
I Y Y S G S T;
SEQ ID NO: 343
GCG AGA CAA GGG AGG TGG GAG CGA GAA AAC TTT GAC TAC;
SEQ ID NO: 344
A R Q G R W E R E N F D Y;
SEQ ID NO: 345
CAGGTGCAGCTACAGCAGTGGGGCGCAGGACTATTGAAGCCTTCGGAGACCCTGTCCCTCACCTGCGCTGTCTCTGATGA
GTCCTTCAGTGATTACTACTGGACCTGGATCCGCCAGCCCCCAGGGAAGGGGCTGGAGTGGATTGGGGAAATTACTCATA
GTGGAAGTACCCACTACAACCCGTCCCTCAAGAGCCGAGTCACCCTGTCAGTTGACACGTCCAAGAACCACTTCTCCCTG
AGCCTCAACTCTGTGACCGCCGCGGACACGGCTATTTATTACTGTGCGAGAGGCGGTGACTACGGTGGTTTACTTGACTA
CTGGGGCCAGGGAACCCTGGTCACCGTCTCCTCA;
SEQ ID NO: 346
QVQLQQWGAGLLKPSETLSLTCAVSDESFSDYYWTWIRQPPGKGLEWIGEITHSGSTHYNPSLKSRVTLSVDTSKNHFSL
SLNSVTAADTAIYYCARGGDYGGLLDYWGQGTLVTVSS;
SEQ ID NO: 347
GAT GAG TCC TTC AGT GAT TAC TAC;
SEQ ID NO: 348
D E S F S D Y Y;
SEQ ID NO: 349
ATT ACT CAT AGT GGA AGT ACC;
SEQ ID NO: 350
I T H S G S T;
SEQ ID NO: 351
GCG AGA GGC GGT GAC TAC GGT GGT TTA CTT GAC TAC;
SEQ ID NO: 352
A R G G D Y G G L L D Y;
SEQ ID NO: 353
CAGCTGCAGCTGCAGGAGTCGGGCCCAGGACTGGTGAAGCCCTCGGAGACCCTGTCCCTCACCTGCACTGTCTCTGGTGG
CTCCATCAGCAGTAGGAGTCACTACTGGGGCTGGATCCGCCAGCCCCCAGGGAAGGGGCTGGAGTGGATTGGGAGTATCT
ATTATAGTGGGAGCACCTATTACAACCCGTCCCTCAAGAGTCGAGTCACCATATCCGTAGACACGTCCAAGAACCAGTTC
TCCCTGAAGCTGAGCTCTGTGACCGCCGCAGACACGGCTGTGTATTACTGTGCGAGACTTGGCTGGTACGCAGAGGAGGC
TTTTGAAATCTGGGGTCAAGGGACAATGGTCACCGTCTCTTCA;
SEQ ID NO: 354
QLQLQESGPGLVKPSETLSLTCTVSGGSISSRSHYWGWIRQPPGKGLEWIGSIYYSGSTYYNPSLKSRVTISVDTSKNQF
SLKLSSVTAADTAVYYCARLGWYAEEAFEIWGQGTMVTVSS;
SEQ ID NO: 355
GGT GGC TCC ATC AGC AGT AGG AGT CAC TAC;
SEQ ID NO: 356
G G S I S S R S H Y;
SEQ ID NO: 357
ATC TAT TAT AGT GGG AGC ACC;
SEQ ID NO: 358
I Y Y S G S T;
SEQ ID NO: 359
GCG AGA CTT GGC TGG TAC GCA GAG GAG GCT TTT GAA ATC;
SEQ ID NO: 360
A R L G W Y A E E A F E I

### Example 2: Growth Inhibition Assay to Assess the Ability of Anti-PfRH5 Antibodies to Inhibit The In Vitro Invasion of Human Red Blood Cells and Growth of P. falciparum Parasites

In this example, a set of four PfRH5-specific mAbs of the invention were tested alone and in combination in a standard growth inhibition assay with one strain of Plasmodium falciparum (Dd2).

### Experimental Procedure

The P.falciparum strain, Dd2 (BEI Resources) was first synchronized with 5% D-sorbitol following standard protocols at 3-5% hematocrit and 1-2% parasitaemia 20-24 hours prior to the start of the assay. Infected human erythrocytes were prepared at a starting parasitaemia of 0.4-0.7% and 2% hematocrit. Infected erythrocytes were combined with PfRH5-specific or control antibodies, starting at a concentration of 666.67 nM with 1:5 serial dilution for each antibody or antibody combination. All antibodies used were human IgG1. The parasites were grown for 40-48 h until the schizont stage was reached (one complete life cycle). Parasite growth was stopped with three washes of cold PBS. Final parasitaemia was determined by measuring the parasite lactate dehydrogenase (LDH) activity (Miura, H. Zhou, A. Diouf, SE. Moretz, MP. Fay, LH. Miller, LB. Martin, MA. Pierce, RD. Ellis, GED. Mullen, CA. Long. Anti-Apical-Membrane-Antigen-1 antibody is more effective than anti-42-kilodalton-Merozoite-Surface-Protein-1 antibody in inhibiting Plasmodium falciparum growth, as determined by the in vitro growth inhibition assay. Clin Vaccine Immunol. 16, 963-968 (2009). PMID: PMC2708396). Percent growth inhibition is expressed relative to uninfected erythrocytes.

### Results Summary and Conclusions

PfRH5-specific antibodies were produced and tested in vitro in a growth inhibition assay in the laboratory-adapted strain as described above. Table 2-1 shows the maximum percent growth inhibition for a subset of PfRH5-specific antibodies and PfRH5-specific antibody combinations. The individual antibodies and antibody combinations displayed similar percent maximum growth inhibition, ranging from approximately 51-69%.

TABLE 2-1
Summary of maximum growth inhibition
activity of antibodies against PfRH5
from Plasmodium falciparum strain Dd2.*

| | Maximum growth |
|---|---|
| | inhibition (%) |
| mAb/combo mAbs | Dd2 |
| H1H29127P + | 66.33 |
| H1H29100P | |
| H1H29127P + | 51.37 |
| H1H29143P | |
| H1H29127P + | 56.96 |
| H1H29104P | |
| H1H29100P + | 68.70 |
| H1H29143P | |
| H1H29100P + | 54.04 |
| H1H29104P | |
| H1H29143P + | 58.54 |
| H1H29104P | |
| H1H29100P | 65.73 |
| H1H29104P | 54.12 |
| H1H29127P | 56.87 |
| H1H29143P | 56.00 |
| human IgG1 negative | −0.93 |
| control antibody specific | |
| for cat allergy antigen - | |
| Fel d 1 | |

*Maximum growth inhibition of antibodies against PfRH5 relative to uninfected human red blood cells. The growth inhibition assay was performed on P. falciparum (mature trophozoite or early schizont) infected red blood cells at 0.4-0.7% parasitaemia. The antibodies were combined with the infected red blood cells. The parasites were grown for 40-48 hours (the timing is parasite strain-dependent) until the schizont stage was reached. Parasite growth was stopped with three washes of cold PBS. Parasite lactate dehydrogenase (LDH) activity was measured immediately after the washes. Percent growth inhibition was expressed relative to uninfected erythrocytes. The results of one representative assay of the growth inhibition assay are shown above.

### Example 3: Growth Inhibition Assay to Assess the Ability of Anti-PfRH5 Antibodies to Inhibit the In Vitro Invasion of Human Red Blood Cells and Growth of P. falciparum Parasites in Combination with Chloroquine

In this example, a subset of four PfRH5-specific mAbs of the invention were tested alone and in combination with chloroquine (CQ), a commonly-used antimalarial drug in a standard growth inhibition assay with two laboratory strains. One strain of the Plasmodium falciparum parasite, 3D7, is susceptible to chloroquine, while strain 7G8 is resistant to the drug.

### Experimental Procedure

Each P.falciparum strain (BEI Resources) was first synchronized with 5% D-sorbitol following standard protocols at 3-5% hematocrit and 1-2% parasitaemia 20-24 hours prior to the start of the assay. Infected human erythrocytes were prepared at a starting parasitaemia of 0.4-0.7% and 2% hematocrit. Infected erythrocytes were combined with PfRH5-specific or control antibodies starting at a concentration of 666.67 nM with 1:5 serial dilution for each IgG1 antibody and chloroquine at one of two concentrations, 4.91 or 6.58 nM. The two concentrations were selected based on the IC25, 4.91 nM, and IC50, 6.58 nM, of chloroquine with the susceptible 3D7 strain. The parasites were grown for 40-48 h until the schizont stage was reached (one complete life cycle). Parasite growth was stopped with three washes of cold PBS. Final parasitaemia was determined by measuring the parasite lactate dehydrogenase (LDH) activity. Percent growth inhibition was expressed relative to uninfected erythrocytes.

### Results Summary and Conclusions

Table 3-1 shows the maximum percent growth inhibition for each antibody alone and antibody/chloroquine combination. Combining chloroquine with PfRH5-specific antibodies further increased the percent maximum growth inhibition obtained with the antibodies alone in the 3D7 strain. Maximum growth inhibition with antibody alone was approximately 34 to 61%, while the addition of 4.81 nM CQ to the mAb had similar maximum growth inhibition (32 to 51%). The addition of 6.58 nM of CQ to the mAb increased the range of growth inhibition at least 20% to 59 to 75%. On the other hand, the individual antibodies and antibody/drug combinations displayed similar percent maximum growth inhibition with the 7G8 strain (mAb alone: 47-51%; mAb +4.81 nM CQ: 44-53%; mAb+6.58 nM CQ: 30-52%).

TABLE 3-1
Summary of maximum growth
inhibition activity of antibodies
against PfRH5 from various
Plasmodium falciparum strains.*

| | | Max growth | |
|---|---|---|---|
| Chloroquine | | inhibition (%) | |
| Phosphate | mAb | 3D7 | 7G8 |
| N/A | H1H29089P | 61.06 | 51.10 |
| | H1H29100P | 58.02 | 48.85 |
| | H1H29147P2 | 34.93 | 47.34 |
| | H1H29187P2 | 47.58 | 49.30 |
| 4.81 nM | H1H29089P | 51.32 | 48.69 |
| | H1H29100P | 46.64 | 53.76 |
| | H1H29147P2 | 31.45 | 44.22 |
| | H1H29187P2 | 44.53 | 48.70 |
| 6.58 nM | H1H29089P | 75.59 | 52.78 |
| | H1H29100P | 71.21 | 50.67 |
| | H1H29147P2 | 59.11 | 29.96 |
| | H1H29187P2 | 71.20 | 38.95 |

*Maximum growth inhibition of antibodies against PfRH5 relative to uninfected human red blood cells. The growth inhibition assay was performed on P. falciparum- (mature trophozoite or early schizont) infected red blood cells at 0.4-0.7% parasitaemia. The antibodies were combined with the infected red blood cells. The parasites were grown for 40-48 hours (parasite strain dependent) until the schizont stage was reached. Parasite growth was stopped with three washes of cold PBS. Parasite lactate dehydrogenase activity was measured immediately after the washes. Percent growth inhibition was expressed relative to uninfected erythrocytes. The results of one representative assay of the growth inhibition assay are shown above.

### Example 4: Growth Inhibition Assay to Assess the Ability of Anti-PfRH5 Antibodies to Inhibit the In Vitro Invasion of Human Red Blood Cells and Growth of P. falciparum Parasites

In this example, a set of 30 PfRH5-specific mAbs of the invention were tested in a standard growth inhibition assay against a number of common laboratory strains (both susceptible and resistant to various antimalarial drugs) and multidrug resistant clinical lines.

### Experimental Procedure

Each P. falciparum strain (BEI Resources) was first synchronized with 5% D-sorbitol following standard protocols at 3-5% hematocrit and 1-2% parasitaemia 20-24 hours prior to the start of the assay. Infected human erythrocytes were prepared at a starting parasitaemia of 0.4-0.7% and 2% hematocrit. Infected erythrocytes were combined with PfRH5-specific or control IgG1 antibodies starting at a concentration of 666.67 nM with 1:5 serial dilution for each antibody. The parasites were grown for 40-48 h until the schizont stage was reached (one complete life cycle). Parasite growth was stopped with three washes of cold PBS. Final parasitaemia was determined by measuring the parasite lactate dehydrogenase (LDH) activity (Miura et al, Clin Vaccine Immunol. 16:963-968 (2009). PMID: PMC2708396). Percent growth inhibition was expressed relative to uninfected erythrocytes.

### Results Summary and Conclusions

Table 4-1 shows the maximum percent growth inhibition for each of the 30 PfRH5-specific mAbs tested at 666.67 nM. Application of several antibodies resulted in decreased growth in all tested laboratory-adapted and clinical P. falciparum strains.

TABLE 4-1
Summary of maximum growth inhibition activity of antibodies against PfRH5
from various Plasmodium falciparum strains.*

| | Max growth inhibition (%) | | | | | | | | |
|---|---|---|---|---|---|---|---|---|---|
| | | | | W2- | | | FCR- | | |
| mAb | D10 | Dd2 | 7G8 | mef | 3D7 | HB3 | 1/FVO | Cam3.II | RF7 |
| H1H29089P | 82.32 | 71.29 | 70.09 | 66.39 | 67.59 | 59.15 | 65.98 | 56.16 | 28.95 |
| H1H29094P | 35.86 | 41.58 | 29.95 | 34.94 | 14.38 | 45.39 | 51.23 | 26.18 | 15.23 |
| H1H29100P | 81.73 | 64.60 | 66.40 | 67.29 | 61.80 | 62.61 | 66.89 | 52.44 | 17.02 |
| H1H29104P | 55.70 | 61.31 | 35.16 | 47.40 | 34.42 | 39.81 | 67.38 | 50.02 | 23.62 |
| H1H29106P | 74.86 | 53.97 | 55.49 | 48.93 | 57.97 | 48.36 | 54.28 | 51.68 | 27.41 |
| H1H29109P | 44.79 | 42.05 | 39.04 | 37.92 | 32.22 | 32.24 | 56.74 | 35.91 | 8.24 |
| H1H29125P | 75.39 | 51.39 | 46.96 | 64.57 | 51.14 | 52.09 | 67.62 | 42.69 | 20.99 |
| H1H29127P | 74.44 | 64.67 | 61.19 | 53.61 | 63.90 | 59.36 | 61.13 | 47.00 | 25.68 |
| H1H29131P | 63.00 | 68.75 | 50.30 | 63.16 | 50.61 | 59.45 | 71.77 | 50.18 | 26.66 |
| H1H29134P | 50.59 | 39.99 | 34.84 | 26.89 | 40.72 | 28.21 | 41.17 | 28.53 | 16.69 |
| H1H29138P | 57.06 | 74.44 | 57.36 | 66.44 | 58.45 | 51.38 | 84.07 | 48.69 | 51.37 |
| H1H29141P | 50.01 | 65.27 | 54.63 | 56.19 | 48.20 | 49.98 | 74.25 | 49.31 | 32.06 |
| H1H29143P | 79.41 | 62.70 | 61.87 | 58.27 | 60.27 | 60.87 | 67.19 | 53.25 | 28.21 |
| H1H29146P2 | 10.78 | 26.90 | 19.07 | 0.57 | 8.57 | 33.87 | 7.32 | 8.18 | 5.97 |
| H1H29147P2 | 81.44 | 65.31 | 65.20 | 64.71 | 60.27 | 64.85 | 70.32 | 51.93 | 31.44 |
| H1H29149P2 | 66.03 | 50.32 | 41.38 | 42.77 | 44.15 | 40.01 | 57.98 | 36.22 | 14.74 |
| H1H29151P2 | 38.66 | 57.97 | 47.52 | 53.90 | 35.49 | 31.74 | 53.70 | 26.34 | 18.26 |
| H1H29163P2 | 71.83 | 61.17 | 62.62 | 63.66 | 55.62 | 56.45 | 64.52 | 46.72 | 10.73 |
| H1H29166P2 | 70.94 | 60.02 | 54.60 | 61.87 | 53.11 | 54.93 | 69.69 | 55.90 | 19.41 |
| H1H29171P2 | 10.60 | 7.71 | 17.03 | 0.74 | 4.04 | 0.52 | 11.67 | 5.45 | −12.52 |
| H1H29179P2 | 62.50 | 13.49 | 34.11 | 22.88 | 24.24 | 40.86 | 49.01 | 33.41 | −2.99 |
| H1H29183P2 | 6.79 | 0.68 | 17.83 | −0.55 | 5.68 | 4.92 | 16.05 | 8.66 | −6.27 |
| H1H29187P2 | 79.61 | 66.02 | 71.80 | 67.28 | 70.71 | 63.91 | 72.16 | 49.22 | 32.73 |
| H1H29192P2 | 8.70 | −15.16 | 12.42 | 4.35 | −1.03 | 5.62 | 15.10 | −3.38 | −7.69 |
| H1H29196P2 | 14.33 | −1.65 | 23.44 | 17.00 | 6.72 | 26.76 | 21.94 | 6.09 | 3.78 |
| H1H29198P2 | 4.38 | 10.13 | 5.31 | −3.61 | −5.40 | 15.53 | 11.08 | 14.66 | 1.53 |
| H1H29207P2 | 73.90 | 61.30 | 58.68 | 65.21 | 61.84 | 49.30 | 69.23 | 45.30 | 31.06 |
| H1H29209P2 | 72.66 | 38.09 | 49.89 | 39.61 | 48.32 | 52.40 | 19.16 | 47.26 | 11.91 |
| H1H29214P2 | 76.52 | 47.21 | 56.74 | 52.72 | 52.09 | 59.09 | 13.05 | 46.67 | 11.18 |
| H1H29215P2 | 35.80 | 22.79 | 33.23 | 37.23 | 19.97 | 30.11 | 54.03 | 27.22 | 11.28 |
| Human IgG1 | −1.42 | 11.02 | −11.68 | 6.66 | 13.18 | 27.50 | 4.04 | −0.41 | −6.62 |
| negative | | | | | | | | | |
| control | | | | | | | | | |
| antibody | | | | | | | | | |
| specific for cat | | | | | | | | | |
| allergy antigen- | | | | | | | | | |
| Fel d 1 | | | | | | | | | |

*Maximum growth inhibition of antibodies against PfRH5 relative to uninfected human red blood cells. The growth inhibition assay was performed on P. falciparum-(mature trophozoite or early schizont) infected red blood cells at 0.4-0.7% parasitaemia. The antibodies were combined with the infected red blood cells. The parasites were grown for 40-48 hours (parasite strain dependent) until the schizont stage was reached. Parasite growth was stopped with three washes of cold PBS. Parasite lactate dehydrogenase(LDH) activity was measured immediately after the washes. Percent growth inhibition is expressed relative to uninfected erythrocytes. The results of one representative assay of the growth inhibition assay for each P. falciparum strain are shown above.

### Example 5: Biacore Binding Kinetics of Anti-PfRH5 Monoclonal Antibodies Binding to PfRH5ΔNL.his at 25° C. and 37° C.

The binding kinetics of the various anti-PfRH5 antibodies of the present invention were determined in this example.

Equilibrium dissociation constants (K_{D}) for different PfRH5 reagents binding to purified anti-PfRH5 monoclonal antibodies were determined using a real-time surface plasmon resonance based Biacore T200 biosensor. All binding studies were performed in 10 mM HEPES, 150 mM NaCl, 3 mM EDTA, and 0.05% v/v Surfactant Tween-20, pH 7.4 (HBS-ET) running buffer at 25° C. and 37° C. The Biacore CM4 sensor chip surface was first derivatized by amine coupling with the goat anti-human Fcy specific polyclonal antibody (Jackson ImmunoResearch Laboratories, Cat #109-005-098) or rabbit anti-mouse Fc specific polyclonal antibody (GE Healthcare Cat #BR100838) to capture anti-PfRH5 IgG1 monoclonal antibodies. Binding studies were performed on recombinant PfRH5 removing the amino terminus M1-Y139 and including residues K140-Q526 but lacking K247-L295 and T216A and T299A expressed with a C-terminal hexahistidine tag (PfRH5ΔNL.6his). Different concentrations of PfRH5ΔNL.6his (3.125-50 nM; 2-fold serial dilution or 0.48-60 nM; 5-fold serial dilution) were first prepared in HBS-ET running buffer and were injected over anti-human Fcγ or anti-mouse Fc captured anti-PfRH5 monoclonal antibody surface for four minutes at a flow rate of 50 uL/minute, while the dissociation of monoclonal antibody bound PfRH5 reagent was monitored for ten minutes in HBS-ET running buffer. The association rate (kₐ) and dissociation rate (k_{d}) constants were determined by fitting the real-time binding sensorgrams to a 1:1 binding model with mass transport limitation using Scrubber 2.0c curve-fitting software. Binding dissociation equilibrium constant (K_{D}) and dissociative half-life (t1/2) were calculated from the kinetic rates as:

$${K}_{D} ( M ) = \frac{k d}{k a} , and t 1 / 2 ( min ) = \frac{ln ( 2 )}{60 * k d}$$

Kinetics parameters for PfRH5ΔNL.6his binding to different anti-PfRH5 monoclonal antibodies of the invention at 25° C. and 37° C. are shown in Tables 5-1 through 5-2, respectively.

At 25° C., all of the anti-PfRH5 monoclonal antibodies of the invention bound to PfRH5ΔNL.6his with K_{D} values ranging from 4.72 pM to 1.67 nM, as shown in Table 5-1. At 37° C., all of the anti-PfRH5 monoclonal antibodies of the invention bound to PfRH5ΔNL.6his with K_{D} values ranging from 1.10 pM to 1.10 nM, as shown in Table 5-2.

TABLE 5-1
Binding kinetics parameters of PfRH5ΔNL.6his binding to anti-PfRH5
monoclonal antibodies at 25° C.

| | | 100 nM | | | | |
|---|---|---|---|---|---|---|
| | mAb | Ag | | | | |
| | Capture | Bound | kₐ | k_{d} | K_{D} | t½ |
| Antibody | Level (RU) | (RU) | (1/Ms) | (1/s) | (M) | (min) |
| H1H29141P | 62.8 ± 4.0 | 29.9 | 2.75E+06 | 1.30E−05 | 4.72E−12 | 888 |
| H1H29209P2 | 152.6 ± 0.6 | 79.5 | 1.54E+06 | 1.37E−05 | 9.09E−12 | 843 |
| H1H29125P | 175.4 ± 1.3 | 88.3 | 8.10E+05 | 1.00E−05* | 1.23E−11 | 1155 |
| H1H29138P | 118.1 ± 0.4 | 61.2 | 1.98E+07 | 4.04E−04 | 2.04E−11 | 28.6 |
| H1H29106P | 109.4 ± 4.0 | 49.3 | 6.31E+06 | 9.93E−04 | 1.57E−10 | 11.6 |
| H1H29134P | 147.7 ± 0.6 | 68.3 | 7.98E+05 | 1.00E−05* | 1.25E−11 | 1155 |
| H1H29109P | 70.4 ± 4.4 | 36.7 | 2.17E+06 | 1.01E−03 | 4.68E−10 | 11.4 |
| H1H29100P | 163.2 ± 1.1 | 83.3 | 2.49E+06 | 1.96E−04 | 7.87E−11 | 59.1 |
| H1H29127P | 177.5 ± 0.3 | 88.1 | 1.91E+06 | 9.12E−04 | 4.76E−10 | 12.7 |
| H1H29089P | 180.4 ± 0.9 | 96 | 1.46E+06 | 3.99E−04 | 2.73E−10 | 29 |
| H1H29094P | 186.1 ± 0.9 | 87.2 | 1.07E+06 | 5.30E−04 | 4.98E−10 | 21.8 |
| H1H29179P2 | 171.7 ± 1.0 | 66.3 | 1.83E+06 | 4.88E−03 | 2.66E−09 | 2.4 |
| H1H29214P2 | 115.5 ± 2.9 | 59.9 | 1.71E+06 | 2.17E−04 | 1.27E−10 | 53.3 |
| H1H29131P | 103.8 ± 5.1 | 52.4 | 3.14E+06 | 2.23E−03 | 7.11E−10 | 5.2 |
| H1H29215P2 | 74.5 ± 0.4 | 34.6 | 1.56E+06 | 1.52E−03 | 9.74E−10 | 7.6 |
| H1H29147P2 | 91.5 ± 4.5 | 43.2 | 3.29E+06 | 1.67E−03 | 5.07E−10 | 6.9 |
| H1H29163P2 | 93.6 ± 0.2 | 38 | 3.02E+06 | 2.00E−03 | 6.63E−10 | 5.8 |
| H1H29187P2 | 108.1 ± 1.3 | 53.2 | 2.18E+06 | 2.19E−04 | 1.01E−10 | 52.8 |
| H1H29149P2 | 195.6 ± 1.6 | 82.9 | 1.51E+06 | 4.14E−04 | 2.74E−10 | 27.9 |
| H1H29207P2 | 204.0 ± 2.0 | 104.9 | 1.68E+06 | 1.72E−03 | 1.03E−09 | 6.7 |
| H1H29104P | 139.4 ± 2.6 | 58.4 | 1.24E+06 | 1.30E−03 | 1.05E−09 | 8.9 |
| H1H29196P2 | 138.3 ± 0.9 | 61 | 1.22E+06 | 2.21E−03 | 1.80E−09 | 5.2 |
| H1H29183P2 | 156.6 ± 1.0 | 23 | 1.59E+05 | 1.00E−05* | 6.28E−11 | 1155 |
| H1H29143P | 110.8 ± 0.3 | 53.4 | 3.41E+06 | 2.77E−03 | 8.13E−10 | 4.2 |
| H1H29166P2 | 102.5 ± 6.6 | 28 | 4.27E+06 | 3.56E−03 | 8.34E−10 | 3.2 |
| H1H29151P2 | 122.4 ± 0.2 | 26 | 1.48E+07 | 2.47E−02 | 1.67E−09 | 0.5 |
| H1H29192P2 | 148.3 ± 0.6 | −0.4 | NB^{$} | NB^{$} | NB^{$} | NB^{$} |
| H1H29198P2 | 121.7 ± 0.3 | −0.8 | NB^{$} | NB^{$} | NB^{$} | NB^{$} |
| H1H29146P2 | 143.2 ± 0.4 | −1.5 | NB^{$} | NB^{$} | NB^{$} | NB^{$} |
| H1H29171P2 | 144.2 ± 0.4 | 0.4 | NB^{$} | NB^{$} | NB^{$} | NB^{$} |
| IgG1 Isotype | 165.3 ± 0.9 | −1.3 | NB^{$} | NB^{$} | NB^{$} | NB^{$} |
| Control | | | | | | |

*indicates that no dissociation of PfRH5ΔNL.6his was observed under the current experimental conditions and the k_{d} value was manually fixed at 1.00E−05 while fitting the data.
^{$}indicates that no binding was observed under the current experimental conditions.
NB means no binding

TABLE 5-2
Binding kinetics parameters of PfRH5ΔNL.6his binding to anti-PfRH5
monoclonal antibodies at 37° C.

| | | 100 nM | | | | |
|---|---|---|---|---|---|---|
| | mAb | Ag | | | | |
| | Capture | Bound | kₐ | k_{d} | K_{D} | t½ |
| Antibody | Level (RU) | (RU) | (1/Ms) | (1/s) | (M) | (min) |
| H1H29141P | 38.8 ± 1.3 | 18.8 | 9.11E+06 | 1.00E−05* | 1.10E−12 | 1155 |
| H1H29209P2 | 66.7 ± 0.8 | 28.6 | 2.11E+06 | 1.00E−05* | 4.74E−12 | 1155 |
| H1H29125P | 65.8 ± 2.6 | 19.9 | 1.58E+06 | 1.00E−05* | 6.34E−12 | 1155 |
| H1H29138P | 40.3 ± 0.2 | 18.7 | 4.26E+07 | 6.87E−04 | 1.61E−11 | 16.8 |
| H1H29106P | 58.6 ± 6.5 | 17.3 | 1.60E+07 | 6.36E−04 | 3.97E−11 | 18.2 |
| H1H29134P | 58.9 ± 0.6 | 19.8 | 2.71E+07 | 1.18E−03 | 4.37E−11 | 9.8 |
| H1H29109P | 40.5 ± 8.0 | 19.7 | 1.20E+07 | 5.51E−04 | 4.58E−11 | 21 |
| H1H29100P | 72.4 ± 1.1 | 33.2 | 4.03E+06 | 2.10E−04 | 5.21E−11 | 55 |
| H1H29127P | 67.3 ± 0.6 | 27.3 | 1.29E+07 | 7.02E−04 | 5.46E−11 | 16.4 |
| H1H29089P | 76.0 ± 1.9 | 32.7 | 4.04E+06 | 2.33E−04 | 5.77E−11 | 49.5 |
| H1H29094P | 72.1 ± 2.1 | 21.2 | 2.05E+06 | 1.19E−04 | 5.79E−11 | 97.2 |
| H1H29179P2 | 79.6 ± 1.1 | 15.3 | 2.14E+07 | 1.26E−03 | 5.88E−11 | 9.2 |
| H1H29214P2 | 59.0 ± 0.7 | 22.9 | 2.70E+06 | 1.63E−04 | 6.04E−11 | 70.9 |
| H1H29131P | 64.8 ± 0.4 | 26.6 | 1.31E+07 | 8.66E−04 | 6.61E−11 | 13.3 |
| H1H29215P2 | 41.9 ± 3.3 | 9.9 | 1.40E+07 | 1.21E−03 | 8.64E−11 | 9.5 |
| H1H29147P2 | 54.6 ± 1.5 | 24.9 | 1.24E+07 | 1.12E−03 | 9.01E−11 | 10.3 |
| H1H29163P2 | 32.7 ± 0.2 | 13 | 1.54E+07 | 1.68E−03 | 1.09E−10 | 6.9 |
| H1H29187P2 | 76.4 ± 8.6 | 29 | 3.19E+06 | 6.87E−04 | 2.16E−10 | 16.8 |
| H1H29149P2 | 117.5 ± 4.1 | 33.9 | 1.49E+06 | 6.47E−04 | 4.36E−10 | 17.9 |
| H1H29207P2 | 111.6 ± 3.9 | 40.8 | 2.61E+06 | 1.23E−03 | 4.72E−10 | 9.4 |
| H1H29104P | 110.3 ± 11.5 | 23 | 1.91E+06 | 9.38E−04 | 4.92E−10 | 12.3 |
| H1H29196P2 | 73.6 ± 1.6 | 14.1 | 2.50E+06 | 2.74E−03 | 1.10E−09 | 4.2 |
| H1H29183P2 | 78.4 ± 1.7 | 3.5 | IC^{#} | IC^{#} | IC^{#} | IC^{#} |
| H1H29143P | 41.5 ± 0.2 | 12.3 | IC^{#} | IC^{#} | IC^{#} | IC^{#} |
| H1H29166P2 | 59.0 ± 1.0 | 23.5 | IC^{#} | IC^{#} | IC^{#} | IC^{#} |
| H1H29151P2 | 45.0 ± 0.2 | 2.7 | NB^{$} | NB^{$} | NB^{$} | NB^{$} |
| H1H29192P2 | 60.3 ± 0.8 | −1.2 | NB^{$} | NB^{$} | NB^{$} | NB^{$} |
| H1H29198P2 | 46.4 ± 1.6 | −3.2 | NB^{$} | NB^{$} | NB^{$} | NB^{$} |
| H1H29146P2 | 52.6 ± 0.4 | −2.6 | NB^{$} | NB^{$} | NB^{$} | NB^{$} |
| H1H29171P2 | 52.0 ± 0.3 | −5.6 | NB^{$} | NB^{$} | NB^{$} | NB^{$} |
| IgG1 Isotype | 74.3 ± 1.0 | −1.2 | NB^{$} | NB^{$} | NB^{$} | NB^{$} |
| Control | | | | | | |

*indicates that no dissociation of PfRH5ΔNL.6his was observed under the current experimental conditions and the k_{d} value was manually fixed at 1.00E−05 while fitting the data.
^{$}indicates that no binding was observed under the current experimental conditions.
^{#}indicates that binding was observed under the current experimental conditions, but kinetic values is unfit table.
IC means inconclusive
NB means no binding

### Example 6: Octet Cross-Competition Between Different Anti-PfRH5 Monoclonal Antibodies

Binding competition between a panel of anti-PfRH5 monoclonal antibodies was determined using a real time, label-free bio-layer interferometry assay on the Octet HTX biosensor platform (Pall ForteBio Corp.).

The experiment was performed at 25° C. in 10 mM HEPES, 150 mM NaCl, 3 mM EDTA, 0.05% v/v Surfactant Tween-20, and 1 mg/mL BSA, pH7.4 (HBS-EBT) buffer with the plate shaking at the speed of 1000 rpm. To assess whether two antibodies are able to compete with one another for binding to their respective epitopes on the recombinant PfRH5 removing the amino terminus M1-Y139 and including residues K140-Q526 but lacking K247-L295 and T216A and T299A expressed with a C-terminal hexahistidine tag (PfRH5ΔNL.6his; SEQ ID: 362), around 1.4-2.0 nm of PfRH5ΔNL.6his was first captured onto anti-Penta-His antibody coated Octet biosensor tips (Fortebio Inc, #18-5122) by submerging the biosensor tips for 60 seconds in wells containing 20 μg/mL solution of PfRH5ΔNL.6his. The antigen captured biosensor tips were then saturated with a first anti-PfRH5 monoclonal antibody (subsequently referred to as mAb-1) by dipping into wells containing 50 ug/mL solution of mAb-1 for 3 minutes. Antibodies used were IgG1. The biosensor tips were then subsequently dipped into wells containing 50 g/mL solution of second anti-PfRH5 monoclonal antibody (subsequently referred to as mAb-2) for 3 minutes. The biosensor tips were washed in HBS-ETB buffer in between every step of the experiment. The real-time binding response was monitored during the entire course of the experiment and the binding response at the end of every step was recorded. The response of mAb-2 binding to PfRH5ΔNL.6his pre-complexed with mAb-1 was compared and competitive/non-competitive behavior of different anti-PfRH5 monoclonal antibodies was determined as shown in Table 6-1.

TABLE 6-1
Cross-competition between
anti-PfRH5 monoclonal antibodies.

| | mAb-2 that competes |
|---|---|
| mAb-1 | with mAb-1 |
| H1H29127P | H1H29106P |
| | H1H29134P |
| H1H29106P | H1H29127P |
| | H1H29134P |
| H1H29134P | H1H29106P |
| | H1H29127P |
| H1H29143P | H1H29187P2 |
| H1H29187P2 | H1H29143P |
| | H1H29151P2 |
| H1H29183P2 | No mAb |
| H1H29104P | No mAb |
| H1H29207P2 | H1H29109P |
| | H1H29147P2 |
| | H1H29166P2 |
| | H1H29171P2 |
| | H1H29163P2 |
| H1H29109P | H1H29207P2 |
| | H1H29147P2 |
| | H1H29166P2 |
| | H1H29171P2 |
| | H1H29163P2 |
| H1H29147P2 | H1H29207P2 |
| | H1H29109P |
| | H1H29166P2 |
| | H1H29171P2 |
| | H1H29163P2 |
| | H1H29131P |
| H1H29166P2 | H1H29207P2 |
| | H1H29109P |
| | H1H29147P2 |
| | H1H29171P2 |
| | H1H29163P2 |
| | H1H29131P |
| H1H29171P2 | H1H29207P2 |
| | H1H29109P |
| | H1H29147P2 |
| | H1H29166P2 |
| | H1H29163P2 |
| | H1H29131P |
| | H1H29094P |
| H1H29163P2 | H1H29207P2 |
| | H1H29109P |
| | H1H29147P2 |
| | H1H29166P2 |
| | H1H29171P2 |
| | H1H29131P |
| | H1H29094P |
| | H1H29215P2 |
| H1H29131P | H1H29147P2 |
| | H1H29166P2 |
| | H1H29171P2 |
| | H1H29163P2 |
| | H1H29094P |
| | H1H29215P2 |
| H1H29094P | H1H29171P2 |
| | H1H29163P2 |
| | H1H29131P |
| | H1H29215P2 |
| | H1H29151P2 |
| | H1H29138P |
| H1H29215P2 | H1H29163P2 |
| | H1H29131P |
| | H1H29094P |
| | H1H29151P2 |
| | H1H29125P |
| H1H29151P2 | H1H29187P2 |
| | H1H29094P |
| | H1H29215P2 |
| | H1H29198P2 |
| H1H29125P | H1H29215P2 |
| H1H29149P2 | H1H29100P |
| | H1H29209P2 |
| | H1H29179P2 |
| | H1H29214P2 |
| | H1H29089P |
| H1H29100P | H1H29149P2 |
| | H1H29209P2 |
| | H1H29179P2 |
| | H1H29214P2 |
| | H1H29089P |
| H1H29209P2 | H1H29149P2 |
| | H1H29100P |
| | H1H29179P2 |
| | H1H29214P2 |
| | H1H29089P |
| H1H29179P2 | H1H29149P2 |
| | H1H29100P |
| | H1H29209P2 |
| | H1H29214P2 |
| | H1H29089P |
| H1H29214P2 | H1H29149P2 |
| | H1H29100P |
| | H1H29209P2 |
| | H1H29179P2 |
| | H1H29089P |
| H1H29089P | H1H29149P2 |
| | H1H29100P |
| | H1H29209P2 |
| | H1H29179P2 |
| | H1H29214P2 |
| H1H29138P | H1H29094P |
| H1H29141P | No mAb |
| H1H29196P2 | No mAb |
| H1H29198P2 | H1H29151P2 |
| H1H29146P2 | No mAb |

### Example 7: Multicycle Growth Inhibition Assay to Assess the Resultant Parasites after Anti-PfRH5 Antibody Pressure

Plasmodium falciparum RH5 specific antibodies inhibit invasion of human red blood cells assay over multiple replication cycles and do not induce mutations in the PfRH5 gene. Invasion of host erythrocytes is an essential step of the Plasmodium falciparum (P. falciparum) life cycle and of malaria pathology. Multiple antimalarial drugs target the asexual blood stages however, their efficacy is threatened by the appearance of drug resistant strains (Arrow et al., Saving Lives, Buying Time: Economics of Malaria Drugs in an Age of Resistance. National Acamies Press (US). 254-266 (2004); Blasco et al., Antimalarial drug resistance: linking Plasmodium falciparum parasite biology to the clinic. Nature Medicine. 23, 917-928 (2017)). Furthermore, antimalarial drugs display different pharmacokinetic properties. Some antimalarial drugs, such as artemisinin and quinine, are rapidly cleared within one parasite life cycle. On the other hand, hydrophobic and lipophilic antimalarial drugs are eliminated slowly, but they are characterized by different absorption rates depending on the amount of dietary fat consumed (Arrow et al., Saving Lives, Buying Time: Economics of Malaria Drugs in an Age of Resistance. National Acamies Press (US). 254-266 (2004)).

Targeting the reticulocyte-binding protein homolog 5 (RH5) protein with polyclonal (pAb) and monoclonal antibodies (mAb) efficiently blocks parasite invasion of several P. falciparum strains into human erythrocytes in vitro (Wright et al., Structure of malaria invasion protein RH5 with erythrocyte basigin and blocking antibodies. Nature. 515, 427-430 (2014); Galaway et al., P113 is a merozoite surface protein that binds the N-terminus of Plasmodium falciparum RH5. Nature Communications. 8, 14333 (2017)). Targeting the RH5 protein with a single antibody or an antibody cocktail may be necessary to generate opposing selection pressures on the same target. In addition, antibodies could compensate for the short half-life of common antimalarial drugs.

Lastly, the Plasmodium parasite has developed ways to escape the host immune response that tries to block the parasite development such as gene polymorphisms. This genetic diversity is often the result of immune pressure (Renia & Goh, Malaria Parasites: The Great Escape. Front Immunol. 7, 463 (2016). PMC5098170). Whole genome sequencing of more than 300 P. falciparum clinical isolates or laboratory strains identified only 15 non-synonymous PfRH5 SNPs within the possible mAb epitopes, demonstrating the conserved nature and the importance of the protein. Immune pressure on conserved regions of a protein may limit the ability of the parasite to develop escape mechanisms (Bustamante et al., A full-length recombinant Plasmodium falciparum PfRH5 protein induces inhibitory antibodies that are effective across common PfRH5 genetic variants, Vaccine, 31, 373-9 (2013)).

In this example, a set of four (4) RH5-specific mAbs, each with hIgG1, of the invention were tested alone in an escape mutant assay with one strain of Plasmodium falciparum (3D7).

Monoclonal antibodies used were H1H29089P, H1H29100P, H1H29147P2, H1H29187P2 and REGN1932.

Experimental Procedure (include description of relevant cell lines, proteins, reagents, and instrument type and model): The P. falciparum strain, 3D7 (BEI Resources) was grown following standard protocols at 4% hematocrit and 0.5% parasitaemia. Infected erythrocytes were combined with PfRH5-specific or control antibodies at a concentration corresponding to their respective IC50 value on the P. falciparum strain described above. Antibody concentration was gradually increased every 7-14 days, up to a final concentration corresponding to 110X their respective IC50 values. Growth medium containing the antibody was refreshed every 48 hours and fresh blood was added to the culture weekly.

Every week, parasite RNA was extracted by Trizol lysis of infected red blood cells and purified by Qiagen RNeasy kit. Reverse transcription was completed with the High-Capacity cDNA Reverse Transcription Kit (Applied Biosystems). Amplification of the RH5 gene was performed using PfRH5-specific primers. PCR products were analyzed on 1.5% agarose gel and cloned into TOPO TA cloning vector (Life Technologies). Sequencing of RH5 was achieved with M13 forward and reverse sequencing primers.

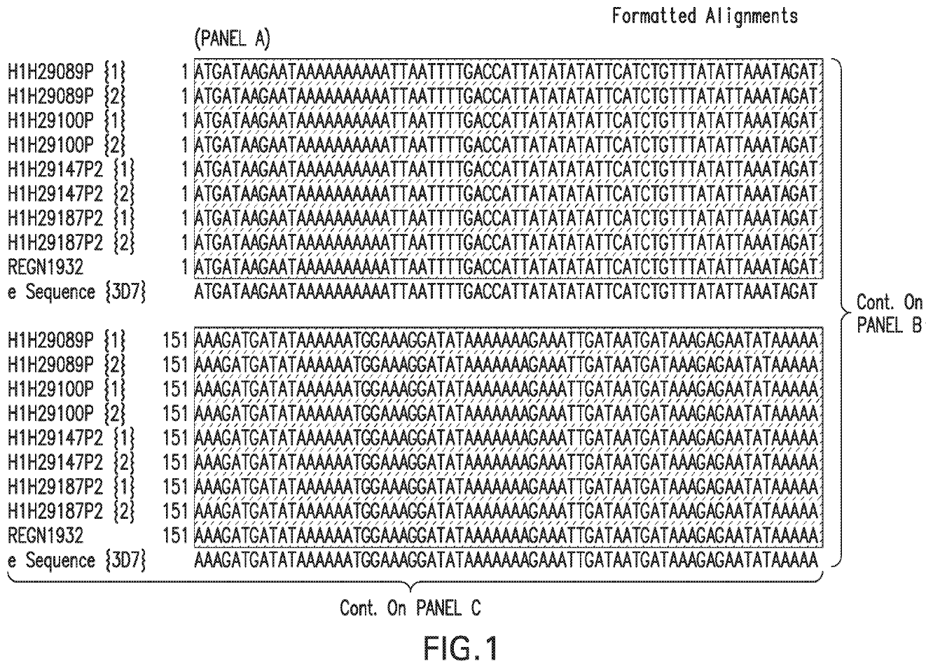

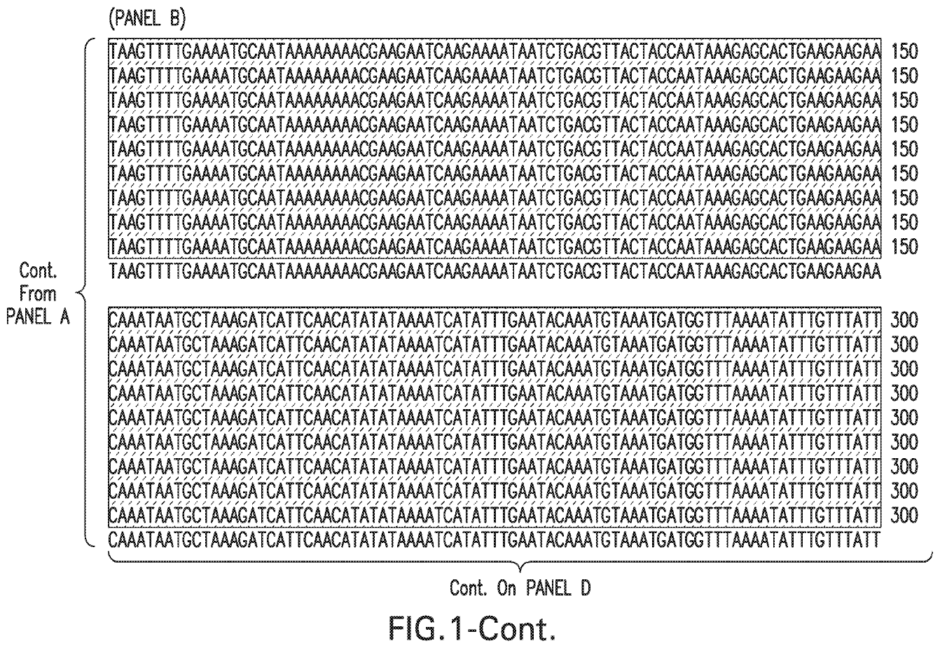

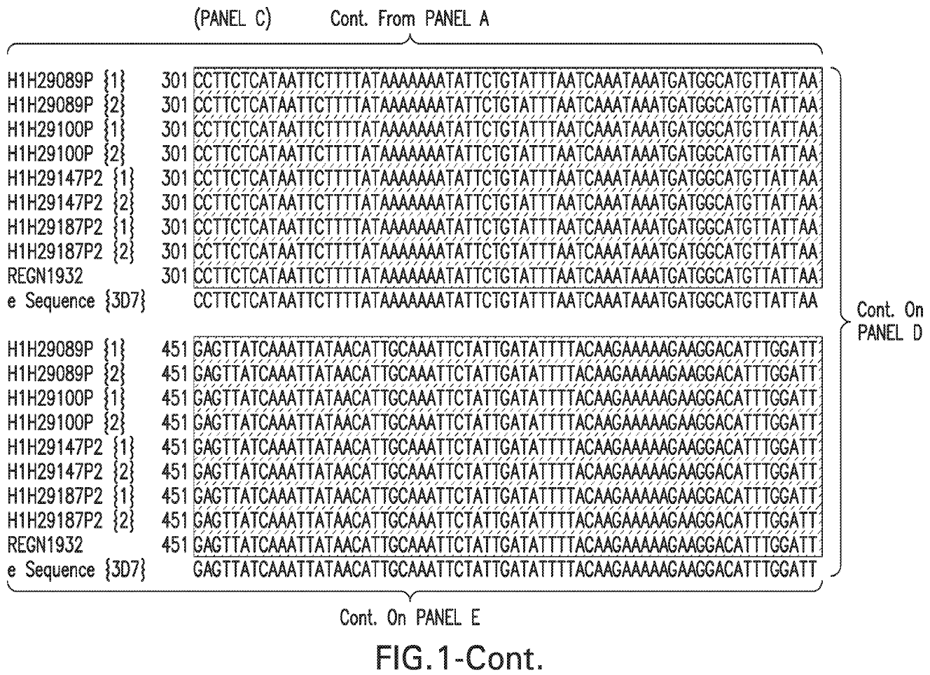

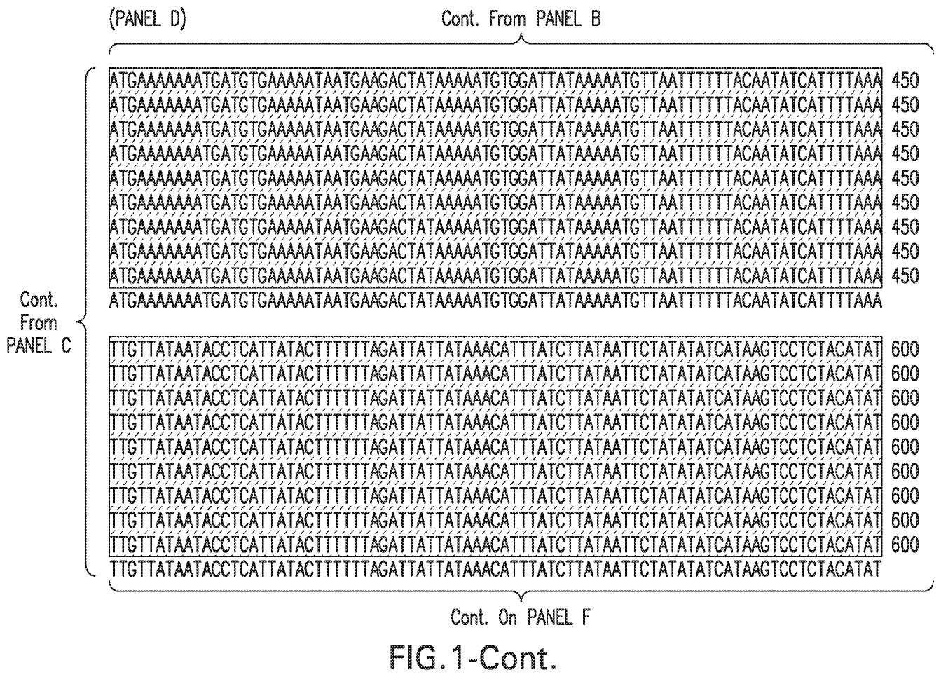

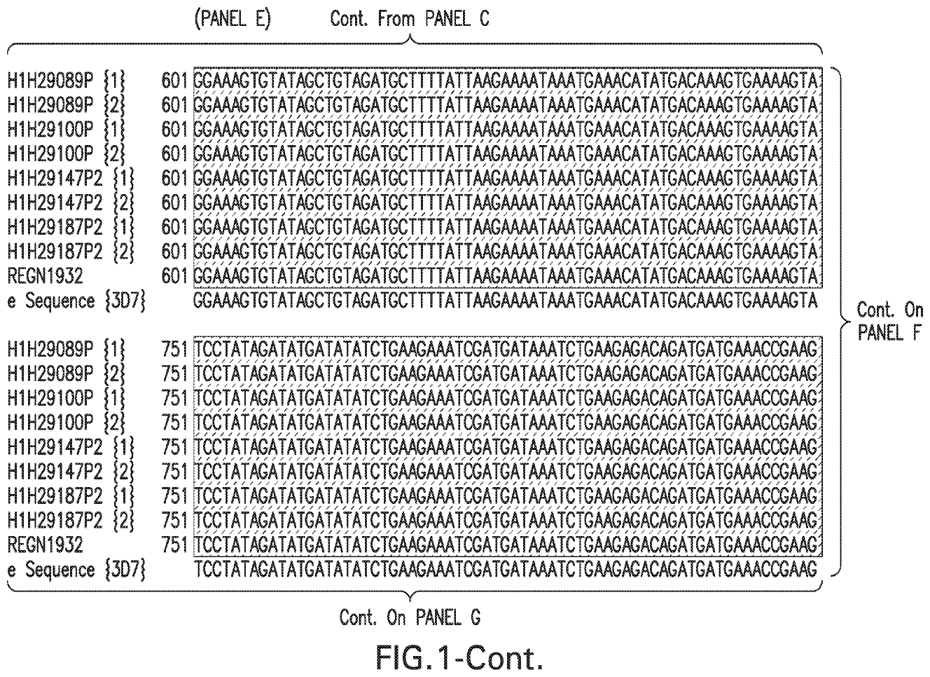

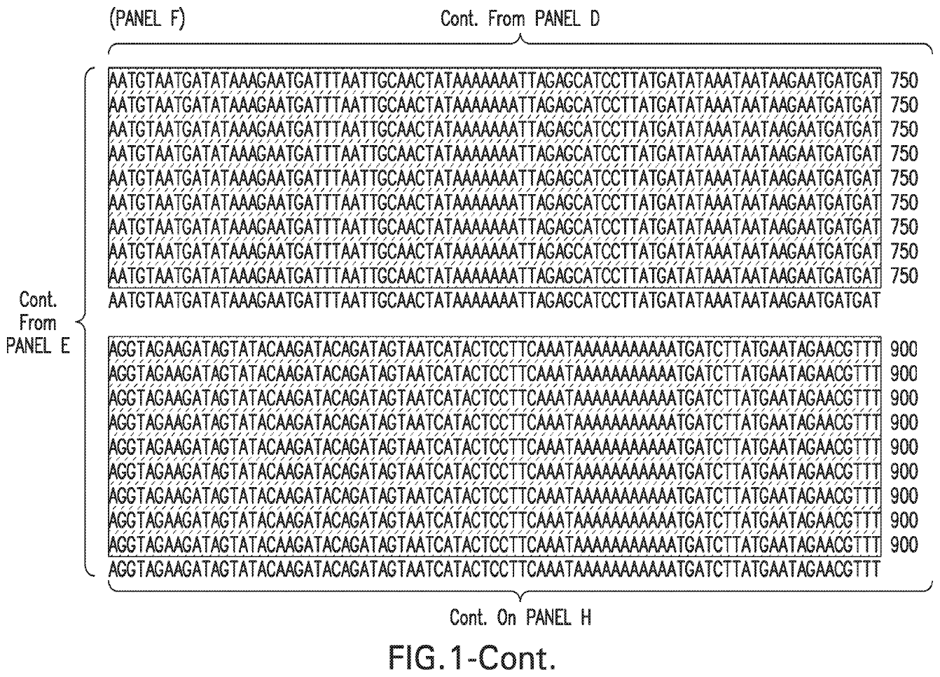

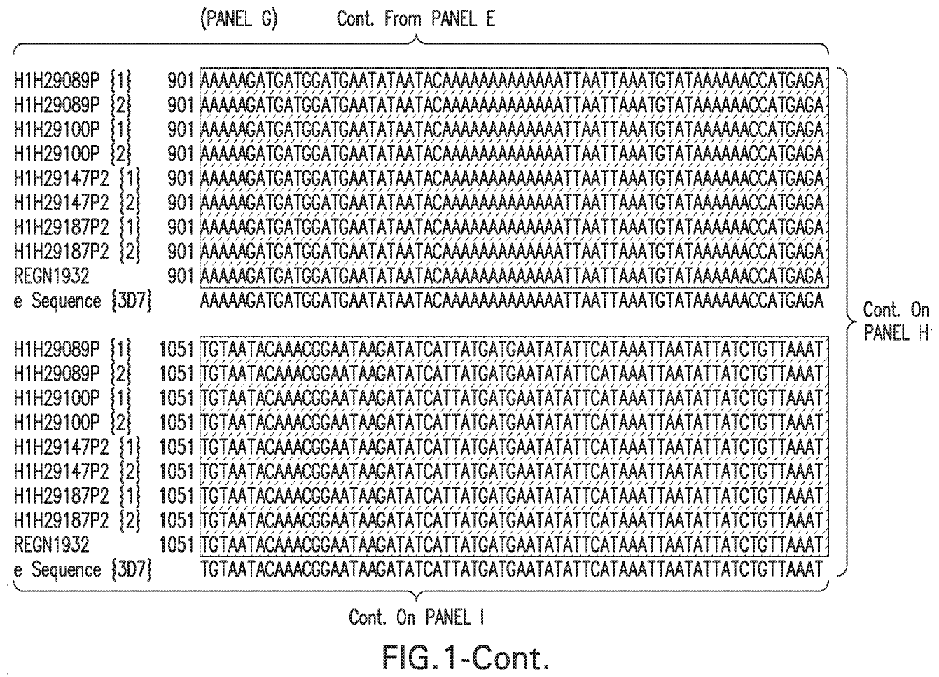

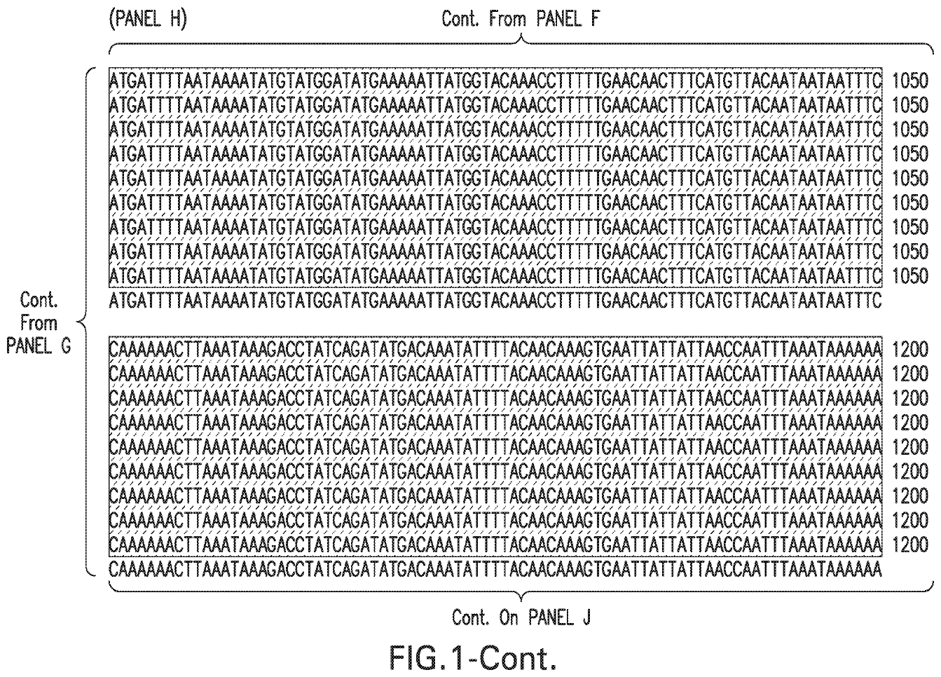

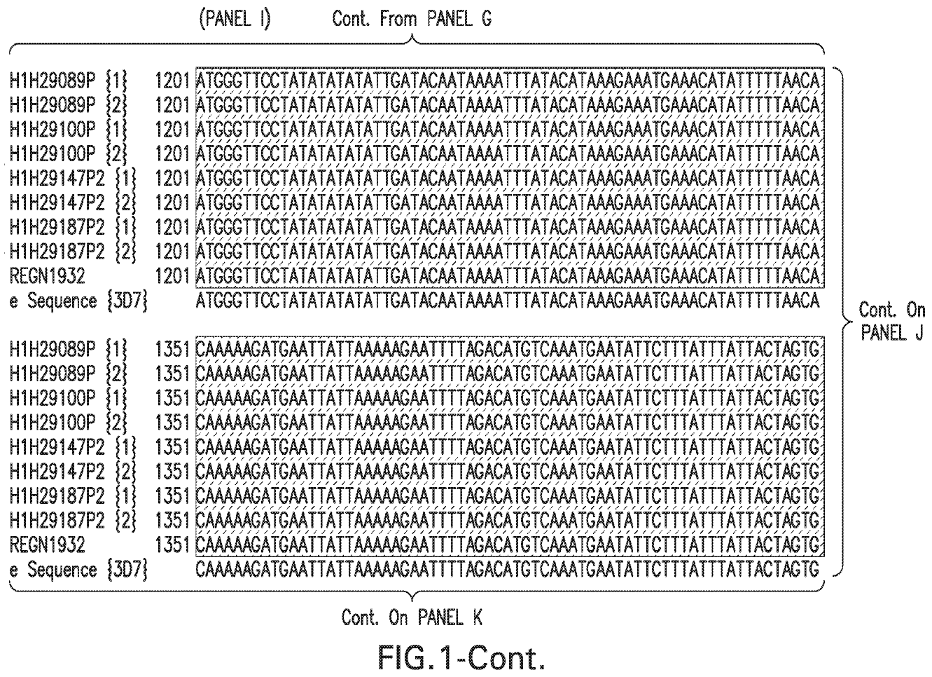

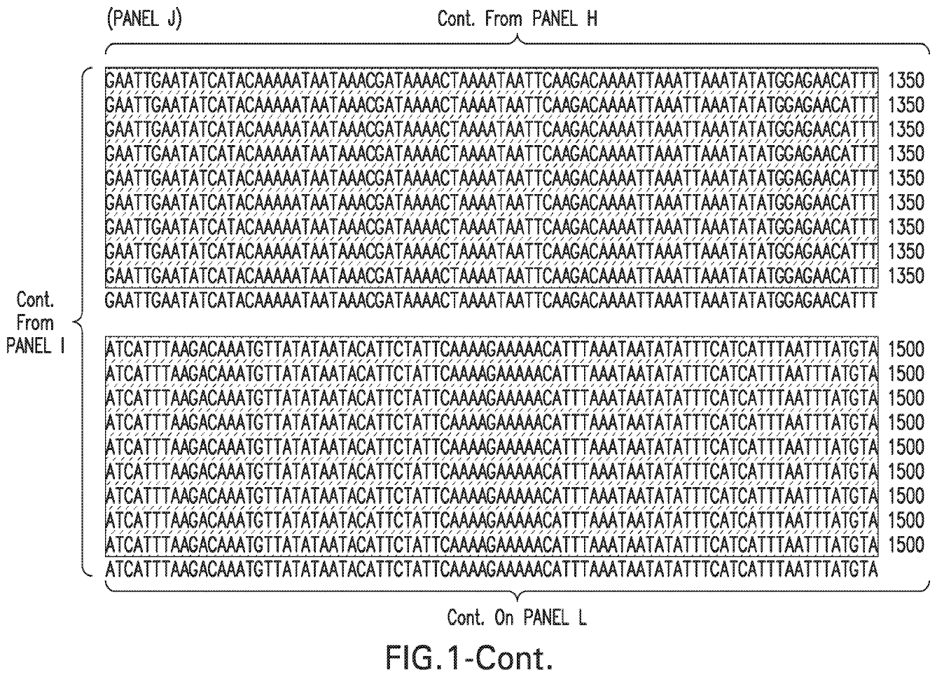

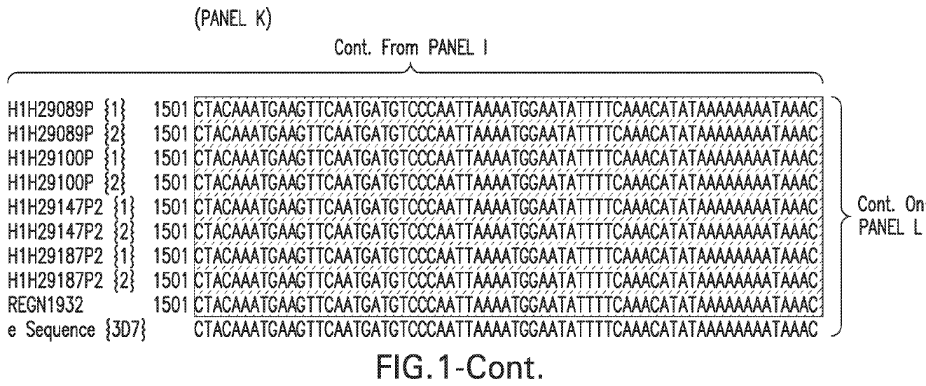

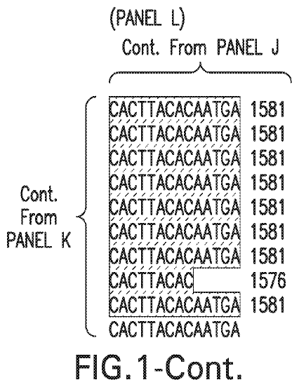

Results summary and conclusions. Several groups have reported that targeting the reticulocyte-binding protein homolog 5 (PfRH5) protein would efficiently block parasite invasion of human erythrocytes in vitro with P. falciparum. Gradually increasing the PfRH5-specific antibody pressure on the P. falciparum 3D7 parasites did not result in PfRH5 polymorphisms compared to the isotype control antibody pressure. Table 7-1 shows the percent PfRH5 sequence identity scores of each sample relative to all other sequenced samples after 45 days of gradual increases in antibody pressure (1×EC₅₀ to 110×EC₅₀). All sequences are 100% identical throughout. FIG. 1 shows the sequence alignments of PfRH5 corresponding to each PfRH5-specific antibody after 45 days of gradual increases in antibody pressure (1×EC₅₀ to 110×EC₅₀) showing no differences in sequences at the nucleotide level.

TABLE 7-1
Percent PfRH5 sequence identity scores of each sample relative to all
other sequenced samples after 45 days of gradual increases in antibody
pressure (1 × EC₅₀ to 110 × EC₅₀).

| | Percent Identity Scores (PfRH5 sequence) | | | | | |
|---|---|---|---|---|---|---|
| | | | | | | Reference |
| | | | | | | sequence |
| | H1H29089P | H1H29100P | H1H29147P2 | H1H29187P2 | REGN1932 | (3D7) |
| H1H29089P | 100 | 100 | 100 | 100 | 100 | 100 |
| H1H29100P | 100 | 100 | 100 | 100 | 100 | 100 |
| H1H29147P2 | 100 | 100 | 100 | 100 | 100 | 100 |
| H1H29187P2 | 100 | 100 | 100 | 100 | 100 | 100 |
| REGN1932 | 100 | 100 | 100 | 100 | 100 | 100 |
| Reference | 100 | 100 | 100 | 100 | 100 | 100 |
| sequence | | | | | | |
| (3D7) | | | | | | |

### Example 8: Growth Inhibition Assay to Assess the Ability of Anti-PfRH5 antibodies to inhibit the in vitro invasion of human red blood cells and growth of P. falciparum parasites in the presence of serum

Plasmodium falciparum RH5 specific antibodies inhibit invasion of human red blood cells in a pLDH based growth inhibition assay in the presence of serum.

One group suggested that complement activation on the merozoite surface enhances the parasite's ability to invade red blood cells (Biryukov et al., Complement and Antibody-mediated Enhancement of Red Blood Cell Invasion and Growth of Malaria Parasites. EBioMedicine. 9, 207-216 (2016)). However, other studies indicate that the presence of complement active serum results in reduced or comparable parasite growth compared to complement inactive serum (Boyle et al, Human antibodies fix complement to inhibit Plasmodium falciparum invasion of erythrocytes and are associated with protection against malaria. Immunity. 42, 580-90 (2015); Chulay et al., Inhibition of in vitro growth of Plasmodium falciparum by immune serum from monkeys. J Infect Dis. 144, 270-278). Also, in all cases of vaccination of merozoite antigens in humans (or any malaria antigens), there are no documented cases of antibody-dependent increases in parasitemia.

In this example, a set of four (4) RH5-specific mAbs (each with hIgG1 (designated with a H1H prefix) or hIgG4 (designated with a H4H prefix)) of the invention were tested alone and in combination with Aotus monkey normal serum (ANS), Aotus heat-inactivated serum (AHIS), human normal serum (HNS) or human heat-inactivated serum (HHIS) in a standard growth inhibition assay with one strain of Plasmodium falciparum (FCR-1/FVO).

Monoclonal antibodies used were H1H29089P, H1H29100P, H1H29147P2, H1H29187P2, H4H29089P, H4H29100P, H4H29147P2, H4H29187P2, REGN1932 (anti-Fel d1 (human IgG1)) and REGN1945 (anti-Fel d1 (human IgG4)).

Experimental Procedure (include description of relevant cell lines, proteins, reagents, and instrument type and model). The P. falciparum strain, FCR-1/FVO (BEI Resources) was first synchronized with 5% D-sortibol following standard protocols at 3-5% hematocrit and 1-2% parasitaemia 20-24 hours prior to the start of the assay. Infected human erythrocytes were prepared at a starting parasitaemia of 0.4-0.7% and 2% hematocrit. Infected erythrocytes were combined with RH5-specific or control antibodies, at a concentration of 6.67 uM in the presence of 10% Aotus normal serum, Aotus heat-inactivated serum, human normal serum or human heat-inactivated serum. The parasites were grown for 40-48 hours until the schizont stage was reached (one complete life cycle). Parasite growth was stopped with three washes of cold PBS. Final parasitaemia was determined by measuring the parasite lactate dehydrogenase (LDH) activity (Miura et al., Anti-Apical-Membrane-Antigen-1 antibody is more effective than anti-42-kilodalton-Merozoite-Surface-Protein-1 antibody in inhibiting Plasmodium falciparum growth, as determined by the in vitro growth inhibition assay. Clin Vaccine Immunol. 16, 963-968 (2009)). Percent growth inhibition is expressed relative to uninfected erythrocytes.

Results summary and conclusions. Targeting the reticulocyte-binding protein homolog 5 (RH5) protein efficiently blocks parasite invasion of human erythrocytes in vitro with P. falciparum. Conflicting findings have been published about the role of complement in merozoite invasion of red blood cells. RH5-specific antibodies were produced and tested in presence of Aotus or human sera in vitro in a growth inhibition assay in a P. falciparum strain as described above. Table 8-1 shows the maximum percent growth inhibition for each RH5-specific antibody (both hIgG1 and hIgG4 formats) with active or inactive serum complement. The individual antibodies and active or inactive serum combinations displayed similar percent maximum growth inhibition, ranging from approximately 67-86%.

TABLE 8-1
Summary of maximum growth inhibition activity of anti-PfRH5
antibodies, in the presence of 10% Aotus or human serum.

| | Percent Maximum Growth Inhibition | | | | |
|---|---|---|---|---|---|
| | mAb | mAb | mAb | mAb | mAb |
| | (6.67 | (6.67 | (6.67 | (6.67 | (6.67 |
| | μM) + | μM) + | μM) + | μM) + | μM) + |
| | ANS | AHIS | HNS | HHIS | No |
| mAb | (10%) | (10%) | (10%) | (10%) | serum |
| H1H29089P | 75.7 | 84.3 | 85.0 | 85.2 | 85.3 |
| H1H29100P | 73.2 | 82.5 | 82.4 | 81.9 | 83.9 |
| H1H29147P2 | 80.3 | 86.2 | 85.6 | 83.4 | 84.1 |
| H1H29187P2 | 80.1 | 89.4 | 85.9 | 85.4 | 85.3 |
| H4H29089P | 75.7 | 82.4 | 85.3 | 86.2 | 84.8 |
| H4H29100P | 67.7 | 79.8 | 75.5 | 79.6 | 83.9 |
| H4H29147P2 | 67.0 | 76.8 | 73.0 | 75.5 | 82.4 |
| H4H29187P2 | 69.9 | 82.9 | 77.0 | 77.8 | 84.0 |
| REGN1932 | 0 | 0 | 0 | 0 | 0 |
| REGN1945 | 0 | 0 | 0 | 0 | 0 |

Maximum growth inhibition of antibodies against PfRH5 relative to uninfected human red blood cells. The growth inhibition assay was performed on P. falciparum (mature trophozoite or early schizont) infected red blood cells at 0.4-0.7% parasitaemia. The antibodies and sera were combined with the infected red blood cells. The parasites were grown for 40-48 hours (the timing is parasite strain-dependent) until the schizont stage was reached. Parasite growth was stopped with three washes of cold PBS. Parasite lactate dehydrogenase (LDH) activity was measured immediately after the washes. Percent growth inhibition is expressed relative to uninfected erythrocytes. The results of one representative assay of the growth inhibition assay is shown above. ANS: Aotus monkey normal serum, AHIS: Aotus heat-inactivated serum, HNS: Normal human serum, HHIS: Human heat-inactivated serum.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, to relate to each and every individual publication, database entry (e.g., Genbank sequences or GeneID entries), patent application, or patent even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

TABLE 9
Sequences Excluded from
ST.26-Formatted Sequence Listing

| SEQ ID NO: | Sequence |
|---|---|
| 13 | gctgcatcc |
| 14 | AAS |
| 29 | gatgcatcc |
| 30 | DAS |
| 45 | tatgcttcc |
| 46 | YAS |
| 61 | gctgcatcc |
| 62 | AAS |
| 77 | gatgcatcc |
| 78 | DAS |
| 93 | gctgcatcc |
| 94 | AAS |
| 109 | gctgcatcc |
| 110 | AAS |
| 125 | gctgcatcc |
| 126 | AAS |
| 141 | ggtgcatcc |
| 142 | GAS |
| 157 | gctgcatcc |
| 158 | AAS |
| 173 | gatgcatcc |
| 174 | DAS |
| 189 | gatgcatcc |
| 190 | DAS |
| 205 | gctgcatcc |
| 206 | AAS |
| 221 | gctgcatcc |
| 222 | AAS |
| 309 | ggtgcatcc |
| 310 | GAS |

1-31. (canceled)

32. An antigen-binding protein that specifically binds to Plasmodium Falciparum reticulocyte binding protein homologue 5 (PfRH5) polypeptide, wherein the antigen binding protein comprises a heavy chain variable region (HCVR) comprising complementarity determining regions (CDRs) HCDR1, HCDR2 and HCDR3 comprising the amino acid sequences of SEQ ID NOs: 36, 38 and 40, respectively, and a light chain variable region (LCVR) comprising CDRs LCDR1, LCDR2 and LCDR3 comprising the amino acid sequences of SEQ ID NOs: 44, 46 and 48, respectively.

33. The antigen-binding protein of claim 32, which is an antibody or antigen-binding fragment thereof.

34. The antigen-binding protein of claim 32, which is multispecific.

35. The antigen-binding protein of claim 32, which comprises one or more of the following properties:
(a) Inhibits growth of Plasmodium falciparum in human red blood cells;
(b) Inhibits growth of Plasmodium falciparum strain D10, Dd2, 7G8, W2-mef, 3D7, HB3, FCR-1/FVO, Cam3.II or RF7 in human red blood cells;
(c) Binds to PfRH5 polypeptide or an antigenic fragment thereof with a Kp of about 4.72 pM to about 1.67 nM at 25° C. and/or of about 1.10 pM to about 1.10 nM at 37° C. when measured by surface plasmon resonance;
(d) Blocks binding of PfRH5 polypeptide to basigin polypeptide;
(e) Binds to PfRH5 lacking the amino-terminal residues M1-Y139 and including residues K140-Q526 but lacking K247-L295 and having the mutations T216A and T299A;
(f) Causes maximal growth inhibition of Plasmodium falciparum in heat inactivated human or Aotus monkey serum that is about 1-10% higher than that of non-heat-inactivated human or Aotus monkey serum, respectively; and/or
(g) When exposed to said antigen-binding protein, does not induce mutation of PfRH5 in Plasmodium falciparum.

36. A complex comprising an antigen-binding protein of claim 32 bound to a Plasmodium falciparum reticulocyte binding protein homologue 5 (PfRH5) polypeptide.

37. A composition or kit comprising the antigen-binding protein of claim 32.

38. The composition or kit of claim 37 in association with a further therapeutic agent which is an anti-parasitic drug or a vaccine.

39. The composition or kit of claim 38, wherein the further therapeutic agent is selected from the group consisting of: chloroquine, atovaquone, proguanil, artemether, lumefantrine, mefloquine, quinine, quinidine, doxycycline, clindamycin, a vaccine, an anti-malarial vaccine and RTS,S/AS01.

40. A pharmaceutical composition comprising the antigen-binding protein of claim 32 and pharmaceutically acceptable carrier.

41. A vessel or injection device comprising the antigen-binding protein of claim 32.

42. The antigen-binding protein of claim 32, wherein the HCVR comprises the amino acid sequence of SEQ ID NO: 34 and the LCVR comprises the amino acid sequence of SEQ ID NO: 42.
